(19) United States
(12) Patent Application Publication
(10) Pub. No.: US 2014/0011206 A1
(43) Pub. Date: Jan. 9, 2014

(54) DETERMINATION OF OOCYTE QUALITY

(71) Applicants: Keith E. Latham, Okemos, MI (US); Young S. Lee, Frederick, MD (US); Catherine A. VandeVoort, Davis, CA (US)

(72) Inventors: Keith E. Latham, Okemos, MI (US); Young S. Lee, Frederick, MD (US); Catherine A. VandeVoort, Davis, CA (US)

(73) Assignees: THE REGENTS OF THE UNIVERSITY OF CALIFORNIA, Oakland, CA (US); TEMPLE UNIVERSITY - OF THE COMMONWEALTH SYSTEM OF HIGHER EDUCATION, Philadelphia, PA (US)

(21) Appl. No.: 13/984,244

(22) PCT Filed: Feb. 8, 2012
(86) PCT No.: PCT/US12/24290

§ 371 Date: Sep. 24, 2013

Related U.S. Application Data
(60) Provisional application No. 61/441,065, filed on Feb. 9, 2011.

(51) Int. Cl. C12Q 1/68

(52) U.S. Cl. CPC C12Q 1/6881
USPC 435/6.12; 435/7.1; 435/7.92

(57) ABSTRACT

A method for evaluating the quality of mammalian oocytes comprises determining the expression level of one or more of the genes ACPP, AQP11, CCDC126, CLU, CYP11 A1, CYP19A1, EGR3, FN1, FOSL2, GMNN, HRAS, HSD3B2, HS-D17B1, HSD11B2, HSDL1, IGF1, IGFBP4, IGFBP5, IRS1, KCNK3, KLF6, NEK6, SMAD7 or STC1 in a test sample derived from a cumulus cell or granulosa cell associated with the oocyte, and comparing the expression level of said at least one marker gene expression in the sample with the expression level in a control. Differential expression of the gene between the sample and the control is indicative of the quality of the oocyte.

### CROSS-REFERENCE TO RELATED APPLICATION

The benefit of the filing date of U.S. Provisional Patent Application No. 61/441,065, filed Feb. 9, 2011, is hereby claimed. The entire disclosure of the aforesaid application is incorporated herein by reference.

### REFERENCE TO GOVERNMENT GRANT

The invention was supported in part by National Institutes of Health, National Center for Research Resources grants RR15253, RR00169 and RR025880. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Jan. 27, 2012, is named 35926408.txt and is 931,059 bytes in size.

### FIELD OF THE INVENTION

The invention relates to a noninvasive method for assessing oocyte quality.

### BACKGROUND OF THE INVENTION

Life begins in the oocyte. The oocyte accumulates a remarkable and complex macromolecular reservoir that mediates and controls the initial development of each new individual, while at the same time eliminating a range of molecules that promote gametogenesis but would otherwise impede early embryogenesis. Failure to complete these transitions appropriately yields an oocyte with limited developmental potential, unable to undergo fertilization or activation, or unable to sustain embryogenesis after fertilization and activation.

Only about 25% of spontaneous conceptions in humans develop to term. With assisted reproduction this number declines to 10-15%. The low percentage of successful pregnancies per fertilized oocyte reflects a variety of factors, of which heterogeneity amongst oocytes in the ability to support embryogenesis is a major element. This heterogeneity in oocyte quality reflects the unique and complex nature of the developing oocyte, a process that occurs in an isolated microenvironment (the follicle) in close coordination and cooperation with somatic cells that regulate oogenesis.

The oocyte develops in close coordination with the somatic companion cells, particularly the cumulus oophorous cells, which maintain direct contact with the oocyte via trans-zonal processes and gap junctions until just before ovulation. Through its secretion of several exogenous factors, the oocyte exerts a commanding role in the overall development of the follicle and the overall differentiation of the cumulus cells. In return, the cumulus cells play a key supportive role by providing to the oocyte a range of extracellular and intracellular molecules that sustain oocyte growth, regulate meiotic progression, and serve as essential metabolic precursors, among other functions.

This intimate relationship between the oocyte and attached cumulus cells causes the two cell types to develop and differentiate progressively and in concert with one another. Importantly, the phenotypic state (e.g., gene expression program) of each cell type depends on that of the other; this situation enables the oocyte to instruct the cumulus cells to provide what it requires at any given time, and as a result, the specific cellular state of the cumulus cell is related to the specific differentiated state of the oocyte. Due to this critical and dynamic relationship, disruption in the development of either cell component can compromise oocyte quality. Because cumulus cell phenotype varies with oocyte developmental state, so too must cumulus cell quality vary between follicles. Hence, the cumulus cell phenotypic state for each follicle will be indicative of the quality of each oocyte. Accordingly, clues to the molecular determinants of oocyte quality may be sought by examination of the cumulus cell phenotype.

Meeting this objective will be advantageous as a non-invasive means of evaluating oocyte quality clinically, and moreover could illuminate the mechanisms of successful follicle development and oogenesis. A number of recent studies have aimed to identify cumulus cell markers or follicular markers of oocyte quality. (Wunder, et al. (2005). J Assist Reprod Genet 22(6): 257-64 2005; Zhang, et al. (2005) Fertil Steril 83 Suppl 1:1169-79; Liu, et al. (2006) J Assist Reprod Genet 23(4):191-8.; Paffoni, et al. (2006) J Soc Gynecol Investig 13(3):226-31; Cillo, et al. (2007) Reproduction 134(5):645-50; Wu, et al. (2007) Hum Reprod 22(6):1526-31; Yanaihara, et al. (2007) Fertil Steril 87(2):279-82; Assidi, et al. (2008) Biol Reprod 79(2):209-22; Bettegowda, et al. (2008) Biol Reprod 79(2):301-9; Fujino, et al. (2008) Fertil Steril 89(1):60-5; Hamel, et al. (2008) Hum Reprod 23(5):1118-27; Ledee, et al. (2008) Hum Reprod 23(9):2001-9; Li, et al. (2008) Mol Hum Reprod 14(12):673-8; Anderson, et al. (2009) Reproduction 138(4):629-37; Caixeta, et al. (2009) Reprod Fertil Dev 21(5):655-64; Ireland, et al. (2009) Biol Reprod 80(5):954-64; Lee, et al. (2009) Endocrinology 150(5):2463-71.) However, these markers have not proven to be of high predictive value, and neither have they yielded significant new insight into the underlying molecular processes that drive productive oocyte-cumulus cell interactions and ultimately high oocyte quality.

In vitro maturation (IVM) comprises giving oocytes the initial stimulus to mature in an in vitro environment. IVM has not been very successful in either nonhuman primates or the human. Only a few reports have been published for IVM of human oocytes from non-stimulated cycles in women, leading to term development (Cha et al., (1991) Fertil. Steril., 55:109-113; Trounson et al., (1994) Fertil. Steril., 62::353-362; Barnes et al., (1995) Hum. Reprod., 10:3243-3247; Russell et al., (1997) Fertil. Steril., 67:616-620; Yoon et al., (2001) J Assist Reprod Genet., 18(6):325-9). Oocyte viability and embryonic development after IVM is substantially lower than with in vivo maturation (VVM). Often, oocytes are retrieved from patients either after failure to mature subsequent to in vivo stimulation or after a low-level dose of human chorionic gonadotropin to initiate the process in vivo (e.g., Chian et al., (2009) Fertil Steril. 91(2):372-6; Yang et al., (2010) Reprod Biomed Online 20(5):656-9). It has been shown that mRNA content with IVM after hCG stimulation in vivo and failure to mature (i.e. oocyte incompetent to mature in vivo were stimulated to mature in vitro) is profoundly altered compared to in vivo matured oocytes (i.e., oocytes that were competent to mature in vivo) (Jones et al., (2008) Hum Reprod. (5):1138-44).

In the rhesus monkey, in vivo oocyte maturation (VVM) yields oocytes of high quality that can support efficient development, whereas in vitro maturation (IVM) consistently yields oocytes of very limited developmental potential (Lee, et al. (2008) Physiol Genomics 35(2):145-58; Nyholt de Prada, et al. (2009) Am J Physiol Endocrinol Metab 296(5):E1049-58). Rhesus monkey oocytes retrieved after 7 days of in vivo follicle stimulating hormone (FSH) stimulation and then signaled to mature in vitro are able to be fertilized and undergo cleavage and in vitro development to blastocyst stage, but long-term development is very limited (Schramm et al., (2003) Hum Reprod., 18(4):826-33)). Modified culture conditions can increase this success (Schramm et al., (2003) supra; Curnow et al., (2010), 25(10): 2465-2474) and affect gene expression in the oocyte (Nyholt de Prada et al., (2010) Mol Reprod Dev. 77(4):353-62), but improvements can be limited to attaining intermediate stages, and successful development after IVM remains limited. Studies indicate that embryos derived from IVM oocytes in the monkey are deficient in the timely onset of embryonic gene transcription (Schramm et al., (2003), supra). Comparisons of mRNA expression profiles between in vivo matured rhesus monkey oocytes and in vitro matured oocytes (no prior hCG stimulation) revealed highly similar expression profiles (Lee, et al. (2008) Physiol Genomics 35(2):145-58).

Collectively, these studies illustrate that oocytes matured by IVM are of reduced quality compared to in vivo matured oocytes.

### SUMMARY OF THE INVENTION

A method for evaluating the quality of a mammalian oocyte comprises: (a) determining the level of expression of at least one marker gene of a set of maker genes comprising ACPP, AQP11, CCDC126, CLU, CYP11A1, CYP19A1, EGR3, FN1, FOSL2, GMNN, HRAS, HSD3B2, HSD17B1, HSD11B2, HSDL1, IGF1, IGFBP4, IGFBP5, IRS1, KCNK3, KLF6, NEK6, SMAD7 and STC1 in a test sample derived from a cumulus cell or granulosa cell associated with the oocyte, after maturation of the oocyte; and (b) comparing the expression level of said at least one marker gene expression in the sample with the expression level in a control, wherein detecting differential expression of the maker gene between the sample and the control is indicative of the quality of the oocyte.

In embodiments, the control comprises a sample derived from a cumulus cell or a granulosa cell associated with a mammalian oocyte of known quality. In some such embodiments, the control comprises a sample derived from a cumulus cell or a granulosa cell associated with an in vitro matured mammalian oocyte. In other embodiments, the control comprises a sample is derived from a cumulus cell or a granulosa cell associated with an in vivo mammalian matured oocyte.

A method for selecting a mammalian oocyte from a plurality of candidate oocytes for preservation or implantation comprises: (a) determining the level of expression of at least one marker gene of a set of marker genes comprising ACPP, AQP11, CCDC126, CLU, CYP11A1, CYP19A1, EGR3, FN1, FOSL2, GMNN, HRAS, HSD3B2, HSD17B1, HSD11B2, HSDL1, IGF1, IGFBP4, IGFBP5, IRS1, KCNK3, KLF6, NEK6, SMAD7 and STC 1 in each of a plurality of samples, each sample being derived from a cumulus cell or a granulosa cell associated with a candidate oocyte; (b) comparing the expression level of said at least one marker gene in the plurality of samples; and (c) selecting for preservation or implantation a candidate oocyte associated with a sample having a level of marker gene expression compared to the level of marker gene expression in other samples, which level of marker gene expression of said selected candidate is indicative of a higher probability of oocyte quality than at least one other oocyte in the plurality of candidate oocytes.

In certain embodiments the level of expression of at least three marker genes is determined. The at least three marker genes may comprise in some embodiments, NEK6, AQP11 and IGF1. In one embodiment, the probability of a mammalian oocyte of high quality (P) is given by the equation:

P=e^{5.608+0.645x+0.100y-2.17z/}1+e^{.}e^{5.608+0.645x+0.100y-2.17z}

wherein:

x is the expression level of NEK6 relative to a control;

y is the expression level of AQP11 relative to a control; and

z is the expression level of IGF1 relative to a control.

In other embodiments, the expression level of at least ten marker genes is determined.

In some embodiments, marker gene expression level is determined by determining the level of mRNA produced from marker genes. For example, the level of mRNA may be determined by reverse transcription polymerase chain reaction. In other embodiments, the marker gene expression level is determined by determining the level of polypeptide produced from marker genes.

In some embodiments, the mammalian oocyte is an oocyte of a domesticated mammal, including but not limited to bovines, goats and pigs. In other embodiments, the mammalian oocyte is an oocyte of a human being.

A kit for evaluating mammalian oocyte quality comprises: a set of reagents that specifically detects the expression levels of one or more marker genes of a mammal comprising ACPP, AQP11, CCDC126, CLU, CYP11A1, CYP19A1, EGR3, FN1, FOSL2, GMNN, HRAS, HSD3B2, HSD17B1, HSD11B2, HSDL1, IGF1, IGFBP4, IGFBP5, IRS1, KCNK3, KLF6, NEK6, SMAD7 or STC1; and instructions for using said kit for evaluating oocyte quality.

In some embodiments, the set of reagents of the kit detects mRNA expressed from one or more marker genes. In some such embodiments, set of reagents comprises nucleic acid probes complementary to mRNA expressed from one or more marker genes. In some embodiments, the nucleic acid probes complementary to mRNA are immobilized on a substrate surface.

In some embodiments, the set of reagents of the kit detects polypeptides encoded by one or more marker genes. In some embodiments, the set of reagents comprises antibodies or aptamers that specifically bind to the polypeptides encoded by one or more marker genes.

In certain embodiments of the methods and kits of the invention for assessment of bovine oocyte quality, the level of at least one of the following mRNAs is determined:

the NEK6 mRNA having Genbank Accession Number NM_{—}001098988.1;

the AQP 11 mRNA having Genbank Accession Number NM_{—}001110069.1;

the CCDC126 mRNA having Genbank Accession Number NM_{—}001082472.2;

the KLF6 mRNA having Genbank Accession Number NM_{—}001035271.2;

the ACPP mRNA having Genbank Accession Number NM_{—}001098866.1;

the IGFBP4 mRNA having Genbank Accession Number NM_{—}174557.3;

the IGF1 mRNA having Genbank Accession Number NM_{—}001077828.1;

the IRS1 mRNA having Genbank Accession Number XM_{—}581382.3;

the IRS1 mRNA having Genbank Accession Number XM_{—}002685642.1;

the FOSL2 mRNA having Genbank Accession Number NM_{—}001192950.1;

the FOSL2 mRNA having Genbank Accession Number XM_{—}002691451.1;

the HRAS mRNA having Genbank Accession Number XM_{—}590626.2;

the HRAS mRNA having Genbank Accession Number XM_{—}874655.1;

the HRAS mRNA having Genbank Accession Number XM_{—}874570.1;

the CLU mRNA having Genbank Accession Number NM_{—}173902.2;

the HSD17B1 mRNA having Genbank Accession Number XM_{—}001253407.1;

the HSD17B1 mRNA having Genbank Accession Number NM_{—}001102365.1;

the HSDL1 mRNA having Genbank Accession Number NM_{—}001098871.1;

the HSD11B2 mRNA having Genbank Accession Number NM_{—}174642.1;

the STC1 mRNA having Genbank Accession Number NM_{—}176669.3;

the CYP11A1 mRNA having Genbank Accession Number NM_{—}176644.2;

the GMNN mRNA having Genbank Accession Number NM_{—}001025337.1;

the CYP19A1 mRNA having Genbank Accession Number NM_{—}174305.1;

the IGFBP5 mRNA having Genbank Accession Number NM_{—}001105327.1;

the KCNK3 mRNA having Genbank Accession Number XM_{—}597401.5;

the KCNK3 mRNA having Genbank Accession Number XM_{—}002691458.1;

the SMAD7 mRNA having Genbank Accession Number NM_{—}001192865.1;

the SMAD7 mRNA having Genbank Accession Number XM_{—}002697763.1;

the SMAD7 mRNA having Genbank Accession Number XM_{—}616030.3;

the EGR3 mRNA having Genbank Accession Number XM_{—}604596.5;

the EGR3 mRNA having Genbank Accession Number XM_{—}002689773.1; and

the FN1 mRNA having Genbank Accession Number NM_{—}001163778.1.

In certain other embodiments of the methods and kits of the invention for assessment of bovine oocyte quality, the level of at least one of the following polypeptides is determined:

the NEK6 polypeptide having Genbank Accession Number NP_{—}001092458.1;

the AQP11 polypeptide having Genbank Accession Number NP_{—}001103539.1;

the CCDC126 polypeptide having Genbank Accession Number NP_{—}001075941.1;

the KLF6 polypeptide having Genbank Accession Number NP_{—}001030348.2;

the ACPP polypeptide having Genbank Accession Number NP_{—}001092336.1;

the IGFBP4 polypeptide having Genbank Accession Number NP_{—}776982.1;

the IGF1 polypeptide having Genbank Accession Number NP_{—}001071296.1;

the IRS1 polypeptide having Genbank Accession Number XP_{—}581382.2;

the IRS1 polypeptide having Genbank Accession Number XP_{—}002685688.1;

the FOSL2 polypeptide having Genbank Accession Number NP_{—}001179879.1;

the FOSL2 polypeptide having Genbank Accession Number XP_{—}002691497.1;

the HRAS polypeptide having Genbank Accession Number XP_{—}590626.2;

the HRAS polypeptide having Genbank Accession Number XP_{—}879748.1;

the HRAS polypeptide having Genbank Accession Number XP_{—}879663.1;

the CLU polypeptide having Genbank Accession Number NP_{—}776327.1;

the HSD17B1 polypeptide having Genbank Accession Number XP_{—}001253408.1;

the HSD17B1 polypeptide having Genbank Accession Number NP_{—}001095835.1;

the HSDL1 polypeptide having Genbank Accession Number NP_{—}001092341.1;

the HSD11B2 polypeptide having Genbank Accession Number NP_{—}777067.1;

the STC1 polypeptide having Genbank Accession Number NP_{—}788842.2;

the CYP11A1 polypeptide having Genbank Accession Number NP_{—}788817.1;

the GMNN polypeptide having Genbank Accession Number NP_{—}001020508.1;

the CYP19A1 polypeptide having Genbank Accession Number NP_{—}776730.1;

the IGFBP5 polypeptide having Genbank Accession Number NP_{—}001098797.1;

the KCNK3 polypeptide having Genbank Accession Number XP_{—}597401.4;

the KCNK3 polypeptide having Genbank Accession Number XP_{—}002691504.1;

the SMAD7 polypeptide having Genbank Accession Number NP_{—}001179794.1;

the SMAD7 polypeptide having Genbank Accession Number XP_{—}002697809.1;

the SMAD7 polypeptide having Genbank Accession Number XP_{—}616030.3;

the EGR3 polypeptide having Genbank Accession Number XP_{—}604596.4;

the EGR3 polypeptide having Genbank Accession Number XP_{—}002689819.1; and

the FN1 polypeptide having Genbank Accession Number NP 001157250.1;

In certain embodiments of the methods and kits of the invention for assessment of human oocyte quality, the level of at least one of the following mRNAs is determined:

the NEK6 mRNA having Genbank Accession Number NM_{—}001145001.2;

the NEK6 mRNA having Genbank Accession Number NM_{—}001166167.1;

the NEK6 mRNA having Genbank Accession Number NM_{—}001166168.1;

the NEK6 mRNA having Genbank Accession Number NM_{—}001166169.1;

the NEK6 mRNA having Genbank Accession Number NM_{—}001166170.1;

the NEK6 mRNA having Genbank Accession Number NM_{—}001166171.1;

the NEK6 mRNA having Genbank Accession Number NM_{—}014397.5;

the AQP11 mRNA having Genbank Accession Number NM_{—}173039.2;

the CCDC126 mRNA having Genbank Accession Number NM_{—}138771.3;

the KLF6 mRNA having Genbank Accession Number NM_{—}001160124.1;

the KLF6 mRNA having Genbank Accession Number NM_{—}001160125.1;

the KLF6 mRNA having Genbank Accession Number NM_{—}001300.5;

the ACPP mRNA having Genbank Accession Number NM_{—}001134194.1;

the ACPP mRNA having Genbank Accession Number NM_{—}001099.4;

the IGFBP4 mRNA having Genbank Accession Number NM_{—}001552.2;

the IGF1 mRNA having Genbank Accession Number NM_{—}000618.3;

the IGF1 mRNA having Genbank Accession Number NM_{—}001111283.1;

the IGF1 mRNA having Genbank Accession Number NM_{—}001111284.1;

the IGF1 mRNA having Genbank Accession Number NM_{—}001111285.1;

the IRS1 mRNA having Genbank Accession Number NM_{—}005544.2;

the FOSL2 mRNA having Genbank Accession Number NM_{—}005253.3;

the HRAS mRNA having Genbank Accession Number NM_{—}001130442.1;

the HRAS mRNA having Genbank Accession Number NM_{—}005343.2;

the HRAS mRNA having Genbank Accession Number NM_{—}176795.3;

the CLU mRNA having Genbank Accession Number NM_{—}001171138.1;

the CLU mRNA having Genbank Accession Number NM_{—}001831.2;

the CLU mRNA having Genbank Accession Number NM_{—}203339.1;

the HSD17B1 mRNA having Genbank Accession Number NM_{—}000413.2;

the HSDL1 mRNA having Genbank Accession Number NM_{—}001146051.1;

the HSDL1 mRNA having Genbank Accession Number NM_{—}031463.4;

the HSD11B2 mRNA having Genbank Accession Number NM_{—}000196.3;

the STC1 mRNA having Genbank Accession Number NM_{—}003155.2;

the CYP11A1 mRNA having Genbank Accession Number NM_{—}000781.2;

the CYP11A1 mRNA having Genbank Accession Number NM_{—}001099773.1;

the GMNN mRNA having Genbank Accession Number NM_{—}015895.3;

the HSD3B2 mRNA having Genbank Accession Number NM_{—}000198.3;

the HSD3B2 mRNA having Genbank Accession Number NM_{—}001166120.1;

the CYP19A1 mRNA having Genbank Accession Number NM_{—}000103.3;

the CYP19A1 mRNA having Genbank Accession Number NM_{—}031226.2;

the IGFBP5 mRNA having Genbank Accession Number NM_{—}000599.3;

the KCNK3 mRNA having Genbank Accession Number NM_{—}002246.2;

the SMAD7 mRNA having Genbank Accession Number NM_{—}001190821.1;

the SMAD7 mRNA having Genbank Accession Number NM_{—}001190822.1;

the SMAD7 mRNA having Genbank Accession Number NM_{—}001190823.1;

the SMAD7 mRNA having Genbank Accession Number NM_{—}005904.3;

the EGR3 mRNA having Genbank Accession Number NM_{—}001199880.1;

the EGR3 mRNA having Genbank Accession Number NM_{—}001199881.1;

the EGR3 mRNA having Genbank Accession Number NM_{—}004430.2;

the FN1 mRNA having Genbank Accession Number NM_{—}002026.2;

the FN1 mRNA having Genbank Accession Number NM_{—}054034.2;

the FN1 mRNA having Genbank Accession Number NM_{—}212474.1;

the FN1 mRNA having Genbank Accession Number NM_{—}212476.1;

the FN1 mRNA having Genbank Accession Number NM_{—}212478.1; and

the FN1 mRNA having Genbank Accession Number NM_{—}212482.1.

In certain other embodiments of the methods and kits of the invention for assessment of human oocyte quality, the level of at least one of the following polypeptides is determined:

the NEK6 polypeptide having Genbank Accession Number NP_{—}001138473.1:

the NEK6 polypeptide having Genbank Accession Number NP_{—}001159639.1;

the NEK6 polypeptide having Genbank Accession Number NP_{—}001159640.1;

the NEK6 polypeptide having Genbank Accession Number NP_{—}001159641.1;

the NEK6 polypeptide having Genbank Accession Number NP_{—}001159642.1;

the NEK6 polypeptide having Genbank Accession Number NP_{—}001159643.1;

the NEK6 polypeptide having Genbank Accession Number NP_{—}055212.2;

the AQP11 polypeptide having Genbank Accession Number NP_{—}766627.1;

the CCDC126 polypeptide having Genbank Accession Number NP_{—}620126.2;

the KLF6 polypeptide having Genbank Accession Number NP_{—}001153596.1;

the KLF6 polypeptide having Genbank Accession Number NP_{—}001153597.1;

the KLF6 polypeptide having Genbank Accession Number NP_{—}001291.3;

the ACPP polypeptide having Genbank Accession Number NP_{—}001127666.1;

the ACPP polypeptide having Genbank Accession Number NP_{—}001090.2;

the IGFBP4 polypeptide having Genbank Accession Number NP_{—}001543.2;

the IGF1 polypeptide having Genbank Accession Number NP_{—}000609.1;

the IGF1 polypeptide having Genbank Accession Number NP_{—}001104753.1;

the IGF1 polypeptide having Genbank Accession Number NP_{—}001104754.1;

the IGF1 polypeptide having Genbank Accession Number NP_{—}001104755.1;

the IRS1 polypeptide having Genbank Accession Number NP_{—}005535.1;

the FOSL2 polypeptide having Genbank Accession Number NP_{—}005244.1;

the HRAS polypeptide having Genbank Accession Number NP_{—}001123914.1;

the HRAS polypeptide having Genbank Accession Number NP_{—}005334.1;

the HRAS polypeptide having Genbank Accession Number NP_{—}789765.1;

the CLU polypeptide having Genbank Accession Number NP_{—}001164609.1;

the CLU polypeptide having Genbank Accession Number NP_{—}001822.2;

the CLU polypeptide having Genbank Accession Number NP_{—}976084.1;

the HSD17B1 polypeptide having Genbank Accession Number NP_{—}000404.2;

the HSDL1 polypeptide having Genbank Accession Number NP_{—}001139523.1;

the HSDL1 polypeptide having Genbank Accession Number NP_{—}113651.4;

the HSD11B2 polypeptide having Genbank Accession Number NP_{—}000187.3;

the STC1 polypeptide having Genbank Accession Number NP_{—}003146.1;

the CYP11A1 polypeptide having Genbank Accession Number NP_{—}000772.2;

the CYP11A1 polypeptide having Genbank Accession Number NP_{—}001093243.1;

the GMNN polypeptide having Genbank Accession Number NP_{—}056979.1;

the HSD3B2 polypeptide having Genbank Accession Number NP_{—}000189.1;

the HSD3B2 polypeptide having Genbank Accession Number NP_{—}001159592.1;

the CYP19A1 polypeptide having Genbank Accession Number NP_{—}000094.2;

the CYP19A1 polypeptide having Genbank Accession Number NP_{—}112503.1;

the IGFBP5 polypeptide having Genbank Accession Number NP_{—}000590.1;

the KCNK3 polypeptide having Genbank Accession Number NP_{—}002237.1;

the SMAD7 polypeptide having Genbank Accession Number NP_{—}001177750.1;

the SMAD7 polypeptide having Genbank Accession Number NP_{—}001177751.1;

the SMAD7 polypeptide having Genbank Accession Number NP_{—}001177752.1;

the SMAD7 polypeptide having Genbank Accession Number NP_{—}005895.1;

the EGR3 polypeptide having Genbank Accession Number NP_{—}001186809.1;

the EGR3 polypeptide having Genbank Accession Number NP_{—}001186810.1;

the EGR3 polypeptide having Genbank Accession Number NP_{—}004421.2;

the FN1 polypeptide having Genbank Accession Number NP_{—}002017.1;

the FN1 polypeptide having Genbank Accession Number NP_{—}473375.2;

the FN1 polypeptide having Genbank Accession Number NP_{—}997639.1;

the FN1 polypeptide having Genbank Accession Number NP_{—}997641.1;

the FN1 polypeptide having Genbank Accession Number NP_{—}997643.1; and

the FN1 polypeptide having Genbank Accession Number NP_{—}997647.1.

In certain embodiments of the methods and kits of the invention for assessment of pig oocyte quality, the level of at least one of the following mRNAs is determined:

the KLF6 mRNA having Genbank Accession Number NM_{—}001134353.2;

the IGFBP4 mRNA having Genbank Accession Number NM_{—}001123129.1;

the IGF1 mRNA having Genbank Accession Number NM_{—}214256.1;

the HRAS mRNA having Genbank Accession Number NM_{—}001044537.1;

the CLU mRNA having Genbank Accession Number NM_{—}213971.1;

the HSD17B1 mRNA having Genbank Accession Number NM_{—}001128472.1;

the HSD11B2 mRNA having Genbank Accession Number NM_{—}213913.1;

the STC1 mRNA having Genbank Accession Number NM_{—}001103212.1;

the CYP11A1 mRNA having Genbank Accession Number NM_{—}214427.1;

the CYP19A1 mRNA having Genbank Accession Number NM_{—}214429.1;

the IGFBP5 mRNA having Genbank Accession Number NM_{—}214099.1;

the SMAD7 mRNA having Genbank Accession Number XM_{—}001927582.1;

the ACPP mRNA having Genbank Accession Number XM_{—}003132419.1;

the IRS1 mRNA having Genbank Accession Number EU681268.1;

the EGR3 mRNA having Genbank Accession Number XM_{—}003132807.1;

the FN1 mRNA having Genbank Accession Number XM_{—}003133641.1;

the FN1 mRNA having Genbank Accession Number XM_{—}003133642.1; and

the FN1 mRNA having Genbank Accession Number XM_{—}003133643.1.

In certain embodiments of the methods and kits of the invention for assessment of pig oocyte quality, the level of at least one of the following polypeptides is determined:

the KLF6 polypeptide having Genbank Accession Number NP_{—}001127825.1;

the IGFBP4 polypeptide having Genbank Accession Number NP_{—}001116601.1;

the IGF1 polypeptide having Genbank Accession Number NP_{—}999421.1;

the HRAS polypeptide having Genbank Accession Number NP_{—}001038002.1;

the CLU polypeptide having Genbank Accession Number NP_{—}999136.1;

the HSD17B1 polypeptide having Genbank Accession Number NP_{—}001121944.1;

the HSD11B2 polypeptide having Genbank Accession Number NP_{—}999078.1;

the STC1 polypeptide having Genbank Accession Number NP_{—}001096682.1;

the CYP11A1 polypeptide having Genbank Accession Number NP_{—}999592.1;

the CYP19A1 polypeptide having Genbank Accession Number NP_{—}999594.1;

the IGFBP5 polypeptide having Genbank Accession Number NP_{—}999264.1;

the SMAD7 polypeptide having Genbank Accession Number XP_{—}001927617.1;

the ACPP polypeptide having Genbank Accession Number XP_{—}003132467.1;

the IRS1 polypeptide having Genbank Accession Number ACG59405.1;

the EGR3 polypeptide having Genbank Accession Number XP_{—}003132855.1;

the FN1 polypeptide having Genbank Accession Number XP_{—}003133689.1;

the FN1 polypeptide having Genbank Accession Number XP_{—}003133690.1; and

the FN1 polypeptide having Genbank Accession Number XP_{—}003133691.1.

As envisioned in the present invention with respect to the disclosed compositions of matter and methods, in one aspect the embodiments of the invention comprise the components and/or steps disclosed herein. In another aspect, the embodiments of the invention consist essentially of the components and/or steps disclosed herein. In yet another aspect, the embodiments of the invention consist of the components and/or steps disclosed herein.

### BRIEF DESCRIPTION OF THE DRAWINGS

FIGS. 1 and 2 are graphs showing the differential RNA expression values observed by gene expression array hybridization analysis and quantitative RT-PCR, respectively, for the indicated genes in cumulus cells associated with in vitro-matured versus in vivo-matured rhesus monkey oocytes. The genes indicated by asterisk (*) are the 24 genes selected as the marker genes according to the present invention.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent depending on the context in which it is used. As used herein, “about” is meant to encompass variations of ±20% or ±10%, more preferably ±5%, even more preferably ±1%, and still more preferably ±0.1%.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies that may be used in the practice of the present invention may exist in a variety of forms including, for example, polyclonal antibodies, monoclonal antibodies, Fv, Fab and F(ab)2, as well as single chain antibodies and humanized antibodies (Harlow et al., 1999, Using Antibodies: A Laboratory Manual, Cold Spring Harbor Laboratory Press, New York; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

A cumulus cell or granulosa cells is “associated” with a particular oocyte if it is contained in or obtained from the same follicle as the particular oocyte.

The term “complementary” refers to nucleic acid sequences that base-pair according to the standard Watson-Crick complementary rules, or that are capable of hybridizing to a particular nucleic acid segment under relatively stringent conditions. Nucleic acid polymers are optionally complementary across only portions of their entire sequences.

The term “control” or “reference standard” describes a material comprising a level of marker expression products of one or more or the marker genes listed herein, such that the control or reference standard may serve as a comparator against which a sample can be compared. By way of non-limiting examples, a control or reference standard may include a sample derived from a cumulus cell or granulosa cell associated with an oocyte of known low (relatively) quality, such as an oocyte that is matured in vitro, or a known low quality oocyte matured in vivo.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Gene expression” or “expression” as used herein refers to the process by which information from a gene is made into a functional gene product, such as RNA or protein. Thus, the “level of expression” of a gene product of a marker gene of the, in a sample of interest, refers to the level of RNA, particularly the level of mRNA, or the level of the encoded protein, and is not intended to be limited to either.

The term “gene” refers to a nucleic acid (e.g., DNA) sequence that comprises coding sequences necessary for the production of a polypeptide, precursor, or RNA (e.g., mRNA, rRNA, tRNA). The term “gene” encompasses both cDNA and genomic forms of a gene.

A genomic form of a gene contains the coding region or “exons” interrupted with non-coding sequences termed “introns” or “intervening regions” or “intervening sequences.” Introns are removed or “spliced out” from the nuclear or primary transcript; introns therefore are absent in the messenger RNA (mRNA) transcript. In addition to containing introns, genomic forms of a gene can also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript).

Both transcription and translation are included in the “gene expression” of the present invention. Accordingly, both of detection at the transcription level (mRNA, cDNA) and detection at the translation level (protein) are included in the determination of marker gene expression level according to the present invention.

The term “hybridization” refers to the process in which two single-stranded nucleic acids bind non-covalently to form a double-stranded nucleic acid; triple-stranded hybridization is also theoretically possible. Complementary sequences in the nucleic acids pair with each other to form a double helix. The resulting double-stranded nucleic acid is a “hybrid.” Hybridization may be between, for example tow complementary or partially complementary sequences. The hybrid may have double-stranded regions and single stranded regions. The hybrid may be, for example, DNA:DNA, RNA:DNA or DNA:RNA. Hybrids may also be formed between modified nucleic acids. One or both of the nucleic acids may be immobilized on a solid support. Hybridization techniques may be used to detect and isolate specific sequences, measure homology, or define other characteristics of one or both strands.

As used herein, an “instructional material” includes a publication, a recording, a diagram, or any other medium of expression, which can be used to communicate the usefulness of the invention in the kit for determining the progression of a disease. The instructional material of the kit of the invention may, for example, be affixed to a container, which contains a reagent of the invention or be shipped together with a container, which contains a reagent. Alternatively, the instructional material may be shipped separately from the container with the intention that the instructional material and the reagent be used cooperatively by the recipient.

By an “in vitro matured mammalian oocyte” is meant an oocyte that is matured outside the body of a mammal. By an “in vivo matured mammalian oocyte” is meant an oocyte that is matured within the body of a mammal.

The term “marker gene” or “marker” as used herein means any of the mammalian genes ACPP, AQP11, CCDC126, CLU, CYP11A1, CYP19A1, EGR3, FN1, FOSL2, GMNN, HRAS, HSD3B2, HSD17B1, HSD11B2, HSDL1, IGF1, IGFBP4, IGFBP5, IRS1, KCNK3, KLF6, NEK6, SMAD7 or STC1, inclusive of their various homologs among mammalian species.

The terms “marker expression” or “marker gene expression” as used herein, encompasses the transcription, translation, post-translation modification, and phenotypic manifestation of a marker gene, including all aspects of the transformation of information encoded in a gene into RNA or protein. By way of non-limiting example, marker expression includes transcription into messenger RNA (mRNA) and translation into protein, as well as transcription into types of RNA such as transfer RNA (tRNA) and ribosomal RNA (rRNA) that are not translated into protein.

By the phrase “determining the level of marker expression” is meant an assessment of the degree of expression of a marker in a sample at the nucleic acid or protein level, using technology available to the skilled artisan to detect a sufficient portion of any marker expression product (including nucleic acids and proteins) of a marker gene, such that the sufficient portion of the marker expression product detected is indicative of the expression of any one of the marker genes.

“Measuring” or “measurement,” or alternatively “detecting” or “detection,” or alternatively “determining” or “determine” means assessing the presence, absence, quantity or amount of either a given substance within a sample, including the derivation of qualitative or quantitative concentration levels of such substances.

Nucleic acids detected or utilized in the practice of the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferably at least 8, 15 or 25 nucleotides in length, but may be up to 50, 100, 1000, or 5000 nucleotides long or a compound that specifically hybridizes to a polynucleotide. Polynucleotides include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). (See U.S. Pat. No. 6,156,501 which is hereby incorporated by reference in its entirety.) The invention also encompasses situations in which there is a nontraditional base pairing such as Hoogsteen base pairing which has been identified in certain tRNA molecules and postulated to exist in a triple helix.

By “oocyte maturation” is meant the resumption of meiosis of a germinal-vesicle intact stage mammalian oocyte, including germinal vesicle breakdown, meiotic progression, extrusion of the first polar body, and subsequent meiotic arrest, e.g., at the metaphase II stage of meiosis (stage of arrest may vary with species), the stage at which the oocyte is normally ovulated and ready for fertilization. This process is known to encompass both nuclear and cytoplasmic changes in the oocyte as well as changes in the cumulus cell phenotype (e.g., cumulus cell expansion).

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. Typical uses of primers include, but are not limited to, sequencing reactions and amplification reactions. A primer is typically single-stranded, but may be double-stranded. Primers are typically deoxyribonucleic acids, but a wide variety of synthetic and naturally-occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., detectable moieties, such as chromogenic, radioactive or fluorescent moieties, or moieties for isolation, e.g., biotin.

As used herein a “probe” is defined as a nucleic acid capable of binding to a target nucleic acid of complementary sequence through one or more types of chemical bonds, usually through complementary base pairing, usually through hydrogen bond formation. A probe specifically hybridizes to a target complementary polynucleotide, but need not reflect the exact complementary sequence of the template. In such a case, specific hybridization of the probe to the target depends on the stringency of the hybridization conditions. As used herein, a probe may include natural (i.e. A, G, U, C, or T) or modified bases (7-deazaguanosine, inosine, etc.). In addition, a linkage other than a phosphodiester bond may join the bases in probes, so long as it does not interfere with hybridization. Thus, probes may be peptide nucleic acids in which the constituent bases are joined by peptide bonds rather than phosphodiester linkages.

As used herein, the “quality” of an oocyte generally means the oocyte's ability to undergo successful fertilization, and then support cleavage, preimplantation development, implantation, and development to birth. A higher quality oocyte is more likely to undergo successful fertilization and term development than a lower quality oocyte.

As used herein, the term “a reagent that specifically detects expression levels” refers to one or more reagents used to detect the expression of one or more genes (e.g., a gene selected from the 24 marker genes provided herein). Examples of suitable reagents include, but are not limited to, nucleic acid probes capable of specifically hybridizing to the gene of interest, PCR primers capable of specifically amplifying the gene of interest, and antibodies capable of specifically binding to proteins expressed by the gene of interest. The term “amplify” is used in the broad sense to mean creating an amplification product. “Amplification”, as used herein, generally refers to the process of producing multiple copies of a desired sequence, particularly those of a sample. A “copy” does not necessarily mean perfect sequence complementarity or identity to the template sequence.

“Sample” or “biological sample” as used herein means a biological material that contains a substance under assay for determination of gene product expression level. The sample may contain any biological material suitable for detecting the desired biomarker, and may comprise cellular and/or non-cellular material.

The term “solid support,” “support,” and “substrate” as used herein are used interchangeably and refer to a material or group of materials having a rigid or semi-rigid surface or surfaces. In one embodiment, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

“Specifically binds” as used herein in the context of an antibody or an aptamer refers to antibody or aptamer binding to a predetermined antigen with a preference that enables the antibody to be used to distinguish the antigen from others to an extent that permits the detection of the target antigens described herein.

### DETAILED DESCRIPTION OF THE INVENTION

We have found that the transcriptomes of IVM and VVM companion cumulus cells display extensive differences related to patterns of gene regulation maturation. We have identified 24 genes (“marker genes”) that are differentially expressed between IVM and VVM cumulus: ACPP, AQP11, CCDC126, CLU, CYP11A1, CYP19A1, EGR3, FN1, FOSL2, GMNN, HRAS, HSD3B2, HSD17B1, HSD11B2, HSDL1, IGF1, IGFBP4, IGFBP5, IRS1, KCNK3, KLF6, NEK6, SMAD7 and STC1. As oocytes matured by IVM are of reduced quality compared to in vivo matured oocytes, the 24 differentially expressed genes provide novel markers of oocyte quality for clinical diagnostics. In particular, analysis of the expression level of one or more of the 24 marker genes in a sample derived from cumulus cells or granulosa cells associated with a particular oocyte provides information on the quality of the oocyte, for purposes of fertilization and/or implantation. Oocytes of high quality are more likely to sustain successful fertilization and implantation; oocytes of low quality, are less likely to sustain successful fertilization and implantation. The assessment is non-invasive and does not harm the oocyte, as the analysis of oocyte quality is obtained indirectly, from cumulus cells or granulosa cells associated with the oocyte under study. The assessment of oocyte quality is performed before fertilization and term development, and reduces the fertilization and subsequent term development of fertilized oocytes that may be of poorer viability. Thus, the assessment of oocyte quality may be performed before implantation, to access the competence of the oocyte for implantation, fertilization or preservation. By “preservation” is meant storage under conditions that will maintain oocyte and/or embryo viability for subsequent use, such as long term storage including cryopreservation.

Unlike expression profiling of oocytes (Lee, et al. (2008) Physiol Genomics 35(2):145-58), cumulus cells associated with in vivo or in vitro maturation reveal a much larger degree of difference, and provide the basis for determining oocyte quality by associated cumulus cell phenotype, without harm to the oocyte.

Without wishing to be bound by any theory, the difference between IVM and VVM is that the last 24 hour of maturation in response to hormonal stimulation in IVM occurs within cumulus-oocyte complexes that have been removed from the follicular environment. Follicular signals during the final 24 hours of maturation must normally modulate cumulus cell and/or oocyte phenotype in a manner that confers high oocyte quality. Developmental competence is acquired in concert with final nuclear and cytoplasmic maturation, which occur at the end of the maturation period. IVM conditions do not adequately support this transition. Hence, oocytes matured by IVM are of reduced quality compared to in vivo matured oocytes.

According to the present invention, marker gene expression level is assayed in samples derived from cumulus cells or granulosa cells associated with an oocyte, after maturation of the oocyte. A sample “derived” from a cumulus or granulosa cell is a composition comprising biological molecules contained in, secreted by or extracted from such cells, which biological molecules may be assayed directly or indirectly (such as following nucleic acid amplification, for example) to determine the level of marker gene expression by said cumulus cell or granulosa cell.

The product of oocyte maturation is a mature egg that has completed nuclear, plasma membrane and cytoplasmic changes that enable normal fertilization and subsequent development to occur. I one embodiment, analysis of oocyte quality according to the present invention is carried out with respect to cumulus cells or granulosa cells associated with an in vivo-matured oocyte. Methods for harvesting pre- and post-maturation oocytes, and their associated cumulus and/or granulosa cells are known to those skilled in the art. For example, trituration with a narrow bore pipette of cumulus-oocyte complexes retrieved from the reproductive tract or microsurgical removal using a small pipette can be used to remove clusters of cumulus cells, creating a cumulus cell “biopsy” of each individual oocyte. RNA is extracted from the removed cumulus cells by known techniques.

Determination of marker gene expression levels in the practice of the inventive method may be performed by any suitable method. For example, marker gene expression level may be determined with respect to expression products comprising RNA (e.g., mRNA) or protein, derived from cumulus cells or granulosa cells associated with an oocyte subject to quality determination. A cumulus cell or granulosa cell is “associated” with a particular oocyte when it is found in the same follicle containing the oocyte.

In one embodiment, cumulus oocyte complexes (COCs) are obtained following in vivo stimulation to maturation. An aliquot of cumulus cells associated with each individual oocyte subject to analysis is removed from the complex by known methods. For example, trituration with a narrow bore pipette of cumulus-oocyte complexes retrieved from the reproductive tract or microsurgical removal using a small pipette can be used to remove clusters of cumulus cells, creating a cumulus cell “biopsy” of each individual oocyte. Alternatively, granulosa cells can be recovered from individual follicular aspirates (e.g., laparoscopically). RNA is extracted from the removed cumulus or granulosa cells by known techniques.

In some embodiments, marker gene expression according to the invention is detected by determining the level of the corresponding mRNA. Methods that may be utilized for determining the level of mRNA expression in a sample are well known in the art and include, but are not limited to, polymerase chain reaction analyses, Northern analyses, and probe arrays. Any RNA isolation technique that does not select against the isolation of mRNA can be utilized for the purification of RNA from pancreatic tissue samples (see, e.g., Ausubel, ed., 1999, Current Protocols in Molecular Biology (John Wiley & Sons, New York). Additionally, large numbers of tissue samples can readily be processed using techniques well known to those of skill in the art, such as, for example, the single-step RNA isolation process of Chomczynski, 1989, U.S. Pat. No. 4,843,155). In some embodiments, the RNA sample may be depleted of one or more RNAs, for example, an RNA sample depleted of rRNA. General methods for total RNA extraction are well known in the art and are disclosed in standard textbooks on molecular biology. Numerous different and versatile kits can be used to extract RNA (i.e., total RNA or mRNA) and are commercially available from, for example, Ambion, Inc. (Austin, Tex.), Amersham Biosciences (Piscataway, N.J.), BD Biosciences Clontech (Palo Alto, Calif.), BioRad Laboratories (Hercules, Calif.), GIBCO BRL (Gaithersburg, Md.), and Giagen, Inc. (Valencia, Calif.). User Guides that describe in great detail the protocol to be followed are usually included in all these kits. The practice of the invention is not limited to any one method of mRNA detection or quantification recited herein, but rather encompasses all presently known or heretofore unknown methods.

In one embodiment, marker gene expression is quantified with respect to a control using a Northern blot analysis. In brief, mRNA is isolated from a given sample using known techniques. The mRNA is then electrophoresed to separate the mRNA species and the mRNA is transferred from the gel to a nitrocellulose membrane. Labeled probes are used to quantify the target marker mRNA.

In another embodiment, marker gene expression is quantified with respect to a control using a Southern blot analysis. Briefly, mRNA is isolated and reverse transcribed to produce cDNA. The cDNA is then optionally digested and run on a gels in buffer and transferred to membranes. Hybridization is then carried out using nucleic acid probes specific for the target mRNA.

The probe that binds the marker gene expression product includes complementary nucleic acids. For example, the nucleic acid reagents may include oligonucleotides (labeled or non-labeled) fixed to a substrate, labeled oligonucleotides not bound with a substrate, pairs of PCR primers, molecular beacon probes, and the like.

The probe may also comprise fragments of nucleotide sequences complementary to the maker gene expression product. A fragment may be defined to be at least about 10 nucleotides (nt), preferably at least about 15 nt, and more preferably at least about 20 nt, still more preferably at least about 30 nt, and even more preferably, at least about 40 nt in length. Such fragments are useful as probes and primers as discussed herein and can be incorporated into kits for use according to the present invention. Of course, larger DNA fragments are also useful according to the present invention.

The nucleotide sequence of the probe molecule may be selected to hybridize to the target marker gene expression product under stringent conditions. The particular hybridization technique is not essential to the invention. Methods for conducting polynucleotide hybridization assays are well known in the art. Any technique commonly used in the art is within the scope of the present invention. Typical probe technology is described in U.S. Pat. No. 4,358,535 to Falkow et al., incorporated by reference herein. For example, hybridization can be carried out in a solution containing 6×SSC (10×SSC: 1.5 M sodium chloride, 0.15 M sodium citrate, pH 7.0), 5Denhardt's (1×Denhardt's: 0.2% bovine serum albumin, 0.2% polyvinylpyrrolidones, 0.02% Ficoll 400), 10 mM EDTA, 0.5% SDS and about 10⁷ cpm of nick-translated DNA for 16 hours at 65° C.

Additionally, if hybridization is to an immobilized nucleic acid, a washing step may be utilized wherein probe binding to polynucleotides of low homology, or nonspecific binding of the probe, may be removed. For example, a stringent wash step may involve a buffer of 0.2×SSC and 0.5% SDS at a temperature of 65° C.

Additional information related to hybridization technology and, more particularly, the stringency of hybridization and washing conditions, may be found in Sambrook et al., Molecular Cloning: A Laboratory Manual, Second Edition, Cold Spring Harbor Laboratory, Cold Spring Harbor, N.Y. (1989), which is incorporated herein by reference.

Hybridization probes generally will comprise at least 15 nucleotides. Preferably, such probes will have at least 30 nucleotides and may have at least 50 nucleotides. In some embodiments, the probes will range between 30 and 50 nucleotides. In some embodiments, the probes will be at least about 50, 60, 70, 80, 90, 100, 110, 120, 130, 140, 150, 160, 170, 180, 190, 200, 210, 220, 230, 240, 250, 260, 270, 280, 290, 300 or more nucleotides in length.

In one embodiment, the hybridized nucleic acids are detected and quantified by one or more labels attached to the sample nucleic acids. The labels may be incorporated by any of a number of means well known to those of skill in the art. In one embodiment, the label is simultaneously incorporated during the amplification step in the preparation of the sample nucleic acids. Thus, for example, PCR with labeled primers or labeled nucleotides will provide a labeled amplification product. In another embodiment, transcription amplification using a labeled nucleotide (e.g. fluorescein-labeled UTP and/or CTP) incorporates a label into the transcribed nucleic acids. In another embodiment PCR amplification products are fragmented and labeled by terminal deoxytransferase and labeled dNTPs. Alternatively, a label may be added directly to the original nucleic acid sample (e.g., mRNA, polyA mRNA, cDNA, etc.) or to the amplification product after the amplification is completed. Means of attaching labels to nucleic acids are well known to those of skill in the art and include, for example, nick translation or end-labeling (e.g. with a labeled RNA) by kinasing the nucleic acid and subsequent attachment (ligation) of a nucleic acid linker joining the sample nucleic acid to a label (e.g., a fluorophore). In another embodiment label is added to the end of fragments using terminal deoxytransferase.

Methods of hybridization signal detection which may be utilized are described, for example, in U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, and in PCT Application PCT/US99/06097 (published as WO99/47964), each of which also is hereby incorporated by reference.

Comparison of gene expression levels according to the methods of the present invention is preferably performed after the gene expression levels obtained have been corrected for both differences in the amount of sample assayed and variability in the quality of the sample used (e.g., amount and quality of mRNA tested). Correction may be carried out by normalizing the levels against reference genes in the same sample. Typically, “housekeeping genes”, such as actin, GAPDH, HPRT, CPB, G6PD, histone H2A, or mitochondrial ribosomal protein S18C, gene are used for this normalization. Alternatively or additionally, normalization can be based on the mean or median signal (e.g., Ct in the case of RT-PCR) of all assayed genes or a large subset thereof (global normalization approach). Expression levels of a marker gene may be normalized by correcting the absolute expression level of a marker by comparing its expression to the expression of a gene that is not a marker, e.g., a housekeeping gene that is constitutively expressed. Suitable genes for normalization include housekeeping genes such as actin, GAPDH, HPRT, CPB, G6PD, histone H2A, or mitochondrial ribosomal protein S18C gene. This normalization allows the comparison of the expression level in one sample, e.g., a test sample to a control sample.

Prior to or concurrent with marker expression analysis, the expression product sample may be amplified using a variety of mechanisms, some of which may employ PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)); transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995); selective amplification of target polynucleotide sequences (U.S. Pat. No. 6,410,276); consensus sequence primed PCR (CP-PCR) (U.S. Pat. No. 4,437,975); arbitrarily primed PCR (AP-PCR) (U.S. Pat. Nos. 5,413,909, 5,861,245); degenerate oligonucleotide primed PCR (DOP-PCR) (Wells et al., 1999, Nuc Acids Res 27:1214-1218); nucleic acid based sequence amplification (NABSA) (See, U.S. Pat. Nos. 5,409,818, 5,554,517, and 6,063,603, each of which is incorporated herein by reference); anchored PCR; competitive PCR (see, for example, U.S. Pat. No. 5,747,251); and rapid amplification of cDNA ends (RACE). Other amplification methods that may be used are described in, U.S. Pat. Nos. 5,242,794, 5,494,810, 4,988,617 and in U.S. Ser. No. 09/854,317.

Additional methods of sample preparation and techniques for reducing the complexity of a nucleic sample are described in Dong et al., Genome Research 11, 1418 (2001), in U.S. Pat. Nos. 6,361,947, 6,391,592 and US Patent Application Publication 20030096235 and US Patent Application Publication 20030082543).

According to one embodiment, mRNA obtained from a sample is reverse-transcribed and the cDNA subjected to a whole transcriptome amplification, for example, using the QUANTITECT Kit (Qiagen, Inc. 27220 Turnberry Lane, Valencia, Calif. 91355) or the OVATION PICO Kit (NuGen, Technologies, Bemmel, The Netherlands). Quantitative real time PCR (qRT-PCR) is performed on, e.g., about 100 ng of cDNA for each sample. qRT-PCR may be performed on a variety of commercially available platforms, such as the Roche LIGHTCYCLER system (Roche Diagnostics, Indianapolis, Ind.) or the ABI STEPONE PLUS instrument, using the manufacturer's recommendations (Applied Biosystems, Foster City, Calif.). Primers for qRT-PCR may be designed based on the sequences of the target nucleic acid and the RT-PCT platform's manufacturers' recommendations and/or instrument requirements.

Alternatively, gene expression levels may be determined by amplifying complementary DNA (cDNA) or complementary RNA (cRNA) produced from mRNA and analyzing it using a microarray. A number of different array configurations and methods of their production are known to those skilled in the art (see, for example, U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637). Microarray technology allows for the measurement of the steady-state mRNA level of a large number of genes simultaneously. Microarrays currently in wide use include cDNA arrays and oligonucleotide arrays. Analyses using microarrays are generally based on measurements of the intensity of the signal received from a labeled probe used to detect a cDNA sequence from the sample that hybridizes to a nucleic acid probe immobilized at a known location on the microarray (see, for example, U.S. Pat. Nos. 6,004,755; 6,218,114; 6,218,122; and 6,271,002). Array-based gene expression methods are known in the art and have been described in numerous scientific publications as well as in patents (see, for example, M. Schena et al., Science, 1995, 270: 467-470; M. Schena et al., Proc. Natl. Acad. Sci. USA 1996, 93: 10614-10619; J. J. Chen et al., Genomics, 1998, 51: 313-324; U.S. Pat. Nos. 5,143,854; 5,445,934; 5,807,522; 5,837,832; 6,040,138; 6,045,996; 6,284,460; and 6,607,885).

Determination of marker gene expression level may also be carried out by quantifying with respect to a control the expressed polypeptide translated from the marker gene transcript. In this case, a protein sample is first prepared from a biological sample, e.g. a cell culture derived, from the cumulus cell or granulosa cell, and the expression of respective proteins is detected. In one embodiment, cellular protein is obtained from cell lysates of cumulus or granulosa cells, and the protein is quantified by standard methods. Any methods available in the art for detecting and quantifying polypeptide encoded by a marker gene according to the invention, is encompassed. Such methods may rely on utilizes a substance comprising a binding moiety for the polypeptide. Assays based on marker protein-specific biomolecule interaction include, but are not limited to, antibody-based assays, aptamer-based assays, receptor and ligand assays, enzyme activity assays, and allosteric regulator binding assays. The invention is not limited to any one method of protein quantification with respect to a control recited herein, but rather encompasses all presently known or heretofore unknown methods, such as methods that are discovered in the art. Proteins may be detected by other methods, e.g., mass spectroscopy analysis, that do not relying on a binding moiety.

In one embodiment, the substance comprises an antibody that specifically binds to a marker protein. Antibodies can be used in various immunoassay-based protein determination methods such as Western blot analysis, immunoprecipitation, radioimmunoassay (RIA), immunofluorescent assay, chemiluminescent assay, flow cytometry, immunocytochemistry and enzyme-linked immunosorbent assay (ELISA).

In an enzyme-linked immunosorbent assay (ELISA), an enzyme such as, but not limited to, horseradish peroxidase (HRP), alkaline phosphatase (AP), beta-galactosidase or urease can be linked, for example, to an antigen antibody or to a secondary antibody for use in a method of the invention. A horseradish-peroxidase detection system may be used, for example, with the chromogenic substrate tetramethylbenzidine (TMB), which yields a soluble product in the presence of hydrogen peroxide that is detectable at 450 nm. Other convenient enzyme-linked systems include, for example, the alkaline phosphatase detection system, which may be used with the chromogenic substrate p-nitrophenyl phosphate to yield a soluble product readily detectable at 405 nm. Similarly, a beta-galactosidase detection system may be used with the chromogenic substrate o-nitrophenyl-beta-D-galactopyranoside (ONPG) to yield a soluble product detectable at 410 nm. Alternatively, a urease detection system may be used with a substrate such as urea-bromocresol purple (Sigma Immunochemicals, St. Louis, Mo.). Useful enzyme-linked primary and secondary antibodies can be obtained from any number of commercial sources.

For chemiluminescence and fluorescence assays, chemiluminescent and fluorescent secondary antibodies may be obtained from any number of commercial sources. Fluorescent detection is also useful for detecting antigen or for determining a level of antigen in a method of the invention. Useful fluorochromes include, but are not limited to, DAPI, fluorescein, Hoechst 33258, R-phycocyanin, B-phycoerythrin, R-phycoerythrin, rhodamine, Texas red and lissamine-Fluorescein- or rhodamine-labeled antigen-specific antibodies.

Radioimmunoassays (RIAs) are described for example in Brophy et al. (1990, Biochem. Biophys. Res. Comm. 167:898-903) and Guechot et al. (1996, Clin. Chem. 42:558-563). Radioimmunoassays are performed, for example, using Iodine-125-labeled primary or secondary antibody.

Quantitative western blotting may also be used to determine the level of marker protein according to the present invention. Western blots are quantified using well known methods such as scanning densitometry (Parra et al., 1998, J. Vasc. Surg. 28:669-675).

A signal emitted from a detectable antibody is analyzed, for example, using a spectrophotometer to detect color from a chromogenic substrate; a radiation counter to detect radiation, such as a gamma counter for detection of Iodine-125; or a fluorometer to detect fluorescence in the presence of light of a certain wavelength. Where an enzyme-linked assay is used, quantitative analysis of the amount of antigen is performed using a spectrophotometer. It is understood that the assays of the invention can be performed manually or, if desired, can be automated and that the signal emitted from multiple samples can be detected simultaneously in many systems available commercially.

The antibody used to determine the level of a marker protein in a sample in an immunnoassay can comprise a polyclonal or monoclonal antibody. The antibody can comprise an intact antibody, or antibody fragments capable of specifically binding a marker protein. Such fragments include, but are not limited to, Fab and F(ab′)₂ fragments.

When the antibody used in the methods of the invention is a polyclonal antibody (IgG), the antibody is generated by inoculating a suitable animal with a marker protein, peptide or a fragment thereof. Antibodies produced in the inoculated animal which specifically bind the marker protein of the invention are then isolated from fluid obtained from the animal. Antibodies may be generated in this manner in several non-human mammals such as, but not limited to goat, sheep, horse, rabbit, and donkey. Methods for generating polyclonal antibodies are well known in the art and are described, for example in Harlow, et al. (1988, In: Antibodies, A Laboratory Manual, Cold Spring Harbor, N.Y.). These methods are not repeated herein as they are commonly used in the art of antibody technology.

When the antibody used in the methods of the invention is a monoclonal antibody, the antibody is generated using any well known monoclonal antibody preparation procedures such as those described, for example, in Harlow et al. (supra) and in Tuszynski et al. (1988), Blood, 72:109-115. Given that these methods are well known in the art, they are not replicated herein. Generally, monoclonal antibodies directed against a desired antigen are generated from mice immunized with the antigen using standard procedures as referenced herein. Monoclonal antibodies directed against full length or peptide fragments of a marker protein of the invention may be prepared using the techniques described in Harlow, et al., supra.

Techniques for detecting and quantifying (such as with respect to a control) antibody binding are well-known in the art. Antibody binding to a marker protein may be detected through the use of chemical reagents that generate a detectable signal that corresponds to the level of antibody binding and, accordingly, to the level of marker protein expression. Examples of such detectable substances include enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, or ³H.

Antibody binding may be detected through the use of a secondary antibody that is conjugated to a detectable label. Examples of detectable labels include but are not limited to polymer-enzyme conjugates. The enzymes in these complexes are typically used to catalyze the deposition of a chromogen at the antigen-antibody binding site, thereby resulting in cell staining that corresponds to expression level of the biomarker of interest. Preferred enzymes of particular interest include horseradish peroxidase (HRP) and alkaline phosphatase (AP).

A protein assay may be employed that combines antibody-protein binding with detection of the reporter nucleic acid by real-time PCR, e.g., TaqMan® Chemistry-Based Protein Assay, Applied BioSystems by Life Technologies Corporation, Carlsbad, Calif. The latter is a proximity ligation assay based upon Fredriksson et al., Nat. Biotechnol. 20:473-477 (2002) and Gullberg et al., Proc Natl Acad Sci USA. 101(22):8420-4 (2204).

Marker proteins of the invention can be detected and quantified by aptamer-based assays, which are very similar to antibody-based assays, but with the use of an aptamer instead of an antibody. An “aptamer-based” assay is thus an assay for the determination of polypeptide that relies on specific binding of an aptamer. An aptamer can be any polynucleotide, generally a RNA or a DNA, which has a useful biological activity in terms of biochemical activity or molecular recognition attributes. Usually, an aptamer has a molecular activity such as having an enzymatic activity or binding to a polypeptide at a specific region (i.e., similar to an epitope for an antibody) of the polypeptide. It is generally known in the art that an aptamer can be made by in vitro selection methods. In vitro selection methods include a well known method called systematic evolution of ligands by exponential enrichment (a.k.a. SELEX). Briefly, in vitro selection involves screening a pool of random polynucleotides for a particular polynucleotide that binds to a biomolecule, such as a polypeptide, or has a particular activity that is selectable. Generally, the particular polynucleotide represents a very small fraction of the pool, therefore, a round of amplification, usually via polymerase chain reaction, is employed to increase the representation of potentially useful aptamers. Successive rounds of selection and amplification are employed to exponentially increase the abundance of a particular aptamer. In vitro selection is described in Famulok, M.; Szostak, J. W., In Vitro Selection of Specific Ligand Binding Nucleic Acids, Angew. Chem. 1992, 104, 1001. (Angew. Chem. Int. Ed. Engl. 1992, 31, 979-988.); Famulok, M.; Szostak, J. W., Selection of Functional RNA and DNA Molecules from Randomized Sequences, Nucleic Acids and Molecular Biology, Vol 7, F. Eckstein, D. M. J. Lilley, Eds., Springer Verlag, Berlin, 1993, pp. 271; Klug, S.; Famulok, M., All you wanted to know about SELEX; Mol. Biol. Reports 1994, 20, 97-107; and Burgstaller, P.; Famulok, M. Synthetic ribozymes and the first deoxyribozyme; Angew. Chem. 1995, 107, 1303-1306 (Angew. Chem. Int. Ed. Engl. 1995, 34, 1189-1192), U.S. Pat. No. 6,287,765, U.S. Pat. No. 6,180,348, U.S. Pat. No. 6,001,570, U.S. Pat. No. 5,861,588, U.S. Pat. No. 5,567,588, U.S. Pat. No. 5,475,096, and U.S. Pat. No. 5,270,163, which are incorporated herein by reference.

Substantially pure marker proteins of the invention, which can be used as an immunogen for raising polyclonal or monoclonal antibodies, or as a substrate for selecting aptamers, can be prepared, for example, by recombinant DNA methods. For example, the cDNA of the marker protein can be cloned into an expression vector by techniques within the skill in the art. An expression vector comprising sequences encoding the maker protein can then be transfected into an appropriate, for example bacterial, host, whereupon the protein is expressed. The expressed protein can then be isolated by any suitable technique.

For example, a marker protein of the invention can be prepared in the form of a bacterially expressed glutathione S-transferase (GST) fusion protein. Such fusion proteins can be prepared using commercially available expression systems, following standard expression protocols, e.g., “Expression and Purification of Glutathione-S-Transferase Fusion Proteins”, Supplement 10, unit 16.7, in Current Protocols in Molecular Biology (1990) and Smith and Johnson, Gene 67: 34-40 (1988); Frangioni and Neel, Anal. Biochem. 210: 179-187 (1993), the entire disclosures of which are herein incorporated by reference.

Assessment of oocyte quality may be carried out by assessing the expression level relative to a control or standard expression level of any one or more of the 24 marker genes described herein. The invention may be practiced by probing the expression level of the complete set of all 24 marker genes, or any 23, 22, 21, 20, 19, 18, 17, 16, 15, 14, 13, 21, 11, 10, 9, 8, 7, 6, 5, 4, 3, 2 or 1 thereof. Accordingly, it should be understood that this description of a range of 24 marker genes should be considered to have specifically disclosed all the possible sub-sets, as well as all individual genes within the set.

In one embodiment, the level or marker gene expression in the sample is at least 20% different from the level or marker gene expression determined for the control, or other sample forming the basis of comparison. In other embodiments, the difference is at least 40%, at least 60%, at least 80%, at least 2-fold, at least 3-fold, at least 4-fold, at least 6-fold, at least 8-fold or at least 10-fold from the control or other sample forming the basis of comparison.

In some embodiments, the expression level of at least three genes is determined. In one embodiment, the at least three genes comprise NEK6, AQP11 and IGF1. For a determination of oocyte quality, in one embodiment the probability, “P”, of a mammalian oocyte of high quality is given by the equation:

P=e^{5.608+0.645x+0.100y-2.17z}/1+e^{.}e^{5.608+0.645x+0.100y-2.17z}

wherein:

x is the expression level of NEK6 relative to a control;

y is the level of AQP11 relative to the control; and

z is the level of IGF1 relative to the control.

In other embodiments, the expression level of at least ten marker genes is determined.

In some embodiments the expression level of any 1, 2, 3, 4, 5, 6, 7, 8, 9, 10, 11, 12, or 13 of the following marker genes is determined: AQP11, CLU, CYP11A1, CYP19A1, FN1, FOSL2, GMNN, HSD17B1, HSD11B2, HSDL1, IRS1, NEK6 and SMAD7.

As demonstrated in the Example that follows, mean candidate marker gene expression values were compared between groups (IVM vs. VVM) adjusted for background using analysis of covariance on rank-transformed expression levels. Pearson correlation coefficients were calculated to evaluate the degree of colinearity and similarity among the expression levels for the various marker genes. Gene expression levels were analyzed for a relationship to oocyte quality. A single gene generalized estimating equation (GEE) was used to calculate the relationship of gene expression to oocyte quality. GEE is a special case of the generalized linear model taking into consideration the occurrence of multiple oocytes per animal in the same analysis. Odds ratios with 95% confidence intervals and P-values were calculated for each gene as each was related to oocyte quality. Univariate GEE analysis demonstrated that the expression of the 24 marker genes ACPP, AQP11, CCDC126, CLU, CYP11A1, CYP19A1, EGR3, FN1, FOSL2, GMNN, HRAS, HSD3B2, HSD17B1, HSD11B2, HSDL1, IGF1, IGFBP4, IGFBP5, IRS1, KCNK3, KLF6, NEK6, SMAD7 and STC1, were significantly related to oocyte quality. See Table 13, “Calculation of Odds Ratios”, below. For IRS 1, the homologous rhesus gene utilized was IRS4.

The odds ratios calculated for each of these 24 genes define the degree of increased or decreased quality per unit change in gene expression. The odds ratios in turn provide for the assignment, for each of the 24 marker genes, of a percent increase in oocyte quality per ACT unit. See Table 1, below. A ACT unit represents CT values, comprising the expression data from RT-PCR, normalized to an internal housekeeping mRNA. One CT unit corresponds to a two-fold difference in gene expression level. Thus, a two-fold difference in expression at either the mRNA or protein level measured by any method would thus reflect a difference in probability of a quality oocyte according to the stated odds ratios.

TABLE 1
Oocyte Quality Odd Ratios

| | | % increase in oocyte |
|---|---|---|
| Gene | Odds ratio | quality per ΔCt unit |
| NEK6 | 1.1926 | 19.26 |
| AQP11 | 1.6286 | 62.86 |
| CCDC126 | 0.8617 | −13.83 |
| KLF6 | 0.7999 | −20.01 |
| ACPP | 0.1608 | −83.92 |
| EGR3 | 0.9108 | −8.92 |
| IGFBP4 | 2.0730 | 107.30 |
| IGF1 | 0.6059 | −39.41 |
| IRS1¹ | 0.3441 | −65.59 |
| FOSL2 | 0.9602 | −3.98 |
| HRAS | 1.1737 | 17.37 |
| CLU | 1.7654 | 76.54 |
| FN1 | 1.1019 | 10.19 |
| HSD17B1 | 2.5095 | 150.95 |
| HSDL1 | 1.1458 | 14.58 |
| HSD11B2 | 1.0912 | 9.12 |
| STC1 | 2.0346 | 103.46 |
| CYP11A1 | 1.1679 | 16.79 |
| GMNN | 1.1579 | 15.79 |
| HSD3B2 | 1.9117 | 91.17 |
| CYP19A1 | 11.2211 | 1022.11 |
| IGFBP5 | 1.0287 | 2.87 |
| KCNK3 | 1.0002 | 0.02 |
| SMAD7 | 1.0329 | 3.29 |

¹Based on the result for the rheses genes IRS4, which is homologous to the human IRS1 gene.

Thus, for each unit of change in ΔCt value, the odds of an oocyte being a quality oocyte increased or decreases according to the odds ratio corresponding to the maker gene. This allows oocytes in a group to be compared to each other in order to select the oocyte(s) of highest relative quality. This also allows comparison of oocytes to a reference sample of known quality in order to judge the quality of the oocytes. In either case, the oocyte quality demonstration provides a probability of the oocyte possessing a desired development potential.

As an example, the data in Table 1 is utilized as follows to determine the relative quality of an oocyte. As between two oocytes differing by one CT unit of NEK6 expression, i.e., a two fold difference in NEK6 expression level, the oocyte with the 2-fold higher level of expression (lower ΔCt) will have a 19.26% lower probability of being a high quality oocyte than the oocyte with the lower NEK6 expression level (higher ΔCt). As between two oocytes differing in NEK6 expression by two CT units, the oocyte characterized by the higher NEK6 expression level (lower ΔCt) will have a 38.52% (19.26%×2) probability of being a lower quality oocyte for developmental purposes than the oocyte characterized by the lower NEK6 expression level (higher ΔCt).

It may be appreciated from a consideration of Table 1 that expression of the marker genes AQP11, CLU, CYP11A1, CYP19A1, FN1, GMNN, HRAS, HSD3B2, HSD17B1, HSD11B2, HSDL1, IGFBP4, IGFBP5, KCNK3, NEK6, SMAD7 and STC1 correlates negatively with oocyte quality, that is, higher expression (lower ΔCt) is indicative of reduced oocyte quality. It may be also appreciated from Table 1 that expression of the marker genes ACPP CCDC126 EGR3, FOSL2, IGF1, IRS1 (based on the result for the homologous rhesus gene IRS4) and KLF6 correlates positively with oocyte quality, that is, higher expression (lower ΔCt) is indicative of increased oocyte quality. Any difference in expression level of a marker may be translated into a corresponding value for relative oocyte quality using the data of Table 1. However, it may be further appreciated that the greater the magnitude of the expression difference in a marker gene as between two oocytes, the larger is the probability that the oocytes will differ in quality.

In certain embodiments, the marker gene is selected from the marker genes characterized by a P-value of less than 0.01 in odds ratio determination of oocyte quality. Those marker genes include: AQP11, CLU, CYP11A1, CYP19A1, FN1, FOSL2, GMNN, HSD17B1, HSD11B2, HSDL1, IRS1, NEK6 and SMAD7. In some embodiments the expression level of any 1, 2, 3, 4, 5, 6, 7, 8, 9, 10, 11, 12, or 13 of the aforementioned marker genes is determined.

Differences in marker gene expression in cumulus cells or granulosa cells associated with a given oocyte, compared to the expression level of cumulus or granulosa cells associated with other oocytes in a group, permits ranking of oocytes in a group of oocytes according to relative quality. Thus, in one embodiment of the invention, a plurality of oocytes from an individual, or group of individuals, are grouped and screened in a single assay; the oocytes characterized with the highest quality probability scores are then selected for fertilization and/or implantation.

In another embodiment of the invention, a test group comprising one or more oocytes for quality probability determination includes one or more reference oocytes of known quality (high or low), and the relative probability of quality for the test oocytes is determined with reference to the control oocyte, applying the relevant percent increase in oocyte quality per ΔCt value from Table 1. Selection of an oocyte characterized by a known quality can be accomplished by several means. Oocytes matured in vivo are of higher quality than those matured in vitro. Oocytes in some species are of differing quality based on time of year (highest quality during breeding season). Oocytes have lower quality with advancing maternal age. Oocyte quality can be reduced in certain genetic mutants. Oocyte quality can be reduced by maternal exposure to a variety of agents. Oocyte quality can be compromised by various in vitro treatments. Oocytes that are grown in vitro from early follicular stages and then matured can be of lower quality. Oocytes that support fertilization, onset of cleavage, faster cleavage rates of embryos, quality blastocyst formation, or term development are of higher quality than those that failed to do so. The cumulus cells from all such classes of oocytes can be used as high- and low-quality reference standards. Synthetic references can also be created by combination of target marker gene sequences or by the in vitro amplification of whole transcriptome cDNA libraries from cumulus cells associated with high or low quality oocytes.

The availability of the panel of marker genes provides for the screening and identification of high or low quality oocytes. The relative expression of any one or combination of marker genes in a cumulus cell or granulosa cell may thus be used to determine the quality of an associated oocyte. These marker genes of the invention can be adopted into a readily applicable PCR-based or protein expression-based assay that may be applied to small clumps of cumulus cells or granulosa cells isolated from individual COCs. The assay would be applicable to human assisted reproduction clinic operations for identifying high quality oocytes for assisted reproduction. This would allow clinical selection of the highest quality oocytes in situations that require fertilized embryos to be transferred, and provide an invaluable means of evaluating oocyte quality after in vitro oocyte culture and IVM, for example in the context of oncofertility programs (Woodruff et al., (2010), Nat Rev. Gin Oncol 7:466-475) or efforts to manipulate GV stage oocytes in emerging assisted reproduction methods involving microsurgery (Li et al., (2001) Mol Reprod Dev 58:180-185; Liu et al., (1999) Hum Reprod 14:2357-2361; Takeuchi et al., (2004) Hum Reprod 19:975-981; Tucker et al., (1998) Hum Reprod 13:3156-3159).

The assay method of the invention also has application to optimizing reproductive biology approaches to enhance production of valuable agricultural species, and in assisted reproduction methods for livestock production in particular.

The assay method of the invention also has application in the assisted reproduction of endangered species, for endangered species preservation.

The assay method of the invention also has application in selection of oocytes for therapeutic cloning.

The assay method of the invention can be used in one application as a research tool to improve IVM conditions, through selection of conditions which yield a marker gene expression profile in cumulus cells and/or granulosa cells that reflect a high quality oocyte. Conversely, conditions that result in a profile indicative of providing a low quality oocyte may be avoided.

While the practice of the present invention has been exemplified using rhesus monkeys, the utility of the invention is not so limited, and extends to determination of oocyte quality in other mammalian species as well, as the marker genes of the invention are evolutionarily conserved and exhibit homology across species. GenBank accession reference numbers for the nucleotide (mRNA) and encoded amino acid sequences of each of the 24 rhesus monkey marker genes is listed in Table 2. The rhesus monkey gene homologus to human IRS 1, (located on human chromosome 2q2) is IRS4. Rhesus monkey gene IRS4 is located on chromosome 12. GenBank accession reference numbers for the homologous human, cattle, mouse and rat sequences are provided in Tables 3, 4, 5 and 6, respectively. Selected homologous marker gene sequences are provided for pig (Table 7) and sheep (Table 8). The nucleotide and amino acid sequence information referenced under the listed GenBank accession numbers is incorporated herein by reference. Where a marker gene exists in more than one known genotype or isoform, the multiple variants are provided. Based on the information in Table 1, and the corresponding known nucleotide and amino acid sequence information for homologous genes in other species (readily obtainable from public sources, e.g., GenBank), the skilled artisan may utilize one or more of the set of 24 marker genes in assessing oocyte quality across mammalian species.

TABLE 2
Rhesus Monkey (Macaca Mulata) Marker Genes

| Gene | Gene ID | Protein ID | mRNA ID |
|---|---|---|---|
| NEK6 | 100427402 | XP_002800024.1 | XM_002799978.1 |
| NEK6 | 100427402 | XP_002800025.1 | XM_002799979.1 |
| AQP11 | 698835 | XP_001090652.1 | XM_001090652.2 |
| CCDC126 | 710357 | NP_001177873.1 | NM_001190944.1 |
| KLF6 | 710784 | XP_001102983.2 | XM_001102983.2 |
| ACPP | 717786 | XP_001115549.1 | XM_001115549.2 |
| IGFBP4 | 700963 | XP_001097914.1 | XM_001097914.2 |
| IGF1 | 698444 | XP_001094251.1 | XM_001094251.2 |
| IGF1 | 698444 | XP_001094016.1 | XM_001094016.2 |
| IGF1 | 698444 | XP_001094129.1 | XM_001094129.2 |
| IRS4 | 707870 | XP_001109882.1 | XM_001109882.2 |
| (homologous to | | | |
| human IRS1) | | | |
| FOSL2 | 703068 | XP_001101118.1 | XM_001101118.2 |
| FOSL2 | 703068 | XP_001101026.1 | XM_001101026.2 |
| HRAS | 698830 | XP_001085804.1 | XM_001085804.2 |
| CLU | 677866 | NP_001182403.1 | NM_001195474.1 |
| HSD17B1 | 710392 | NP_001040597.1 | NM_001047132.1 |
| HSDL1 | 714962 | NP_001181480.1 | NM_001194551.1 |
| HSD11B2 | 574396 | XP_001088119.2 | XM_001088119.2 |
| STC1 | 710782 | XP_001106526.1 | XM_001106526..2 |
| CYP11A1 | 708065 | XP_001096506.2 | XM_001096506.2 |
| GMNN | 708416 | XP_001101599.1 | XM_001101599.2 |
| GMNN | 708416 | XP_002803675.1 | XM_002803629.1 |
| GMNN | 708416 | XP_001101696.1 | XM_001101696.2 |
| GMNN | 708416 | XP_002803676.1 | XM_002803630.1 |
| HSD3B2 | 712686 | XP_001113717.1 | XM_001113717.1 |
| HSD3B2 | 712686 | XP_001113769.1 | XM_001113769.1 |
| HSD3B2 | 712686 | XP_001113744.1 | XM_001113744.1 |
| HSD3B2 | 712686 | XP_002801772.1 | XM_002801726.1 |
| HSD3B2 | 712686 | XP_002801771.1 | XM_002801725.1 |
| CYP19A1 | 678697 | XP_001082665.2 | XM_001082665.2 |
| IGFBP5 | 696339 | XP_001087664.1 | XM_001087664.1 |
| IGFBP5 | 696339 | XP_001087426.1 | XM_001087426.1 |
| KCNK3 | 699287 | XP_001087754.1 | XM_001087754.2 |
| SMAD7 | 698338 | XP_001087560.1 | XM_001087560.2 |
| EGR3 | 709145 | XP_001104840.2 | XM_001104840.2 |
| EGR3 | 709145 | XP_002805331.1 | XM_002805285.1 |
| FN1 | 613269 | XP_001083548.2 | XM_001083548.2 |

TABLE 3
Homo Sapiens Marker Genes

| Gene | Gene ID | Protein ID | SEQ ID NO. | mRNA ID | SEQ ID NO. |
|---|---|---|---|---|---|
| NEK6 | 10783 | NP_001138473.1 | 74 | NM_001145001.2 | 73 |
| NEK6 | 10783 | NP_001159639.1 | 76 | NM_001166167.1 | 75 |
| NEK6 | 10783 | NP_001159640.1 | 78 | NM_001166168.1 | 77 |
| NEK6 | 10783 | NP_001159641.1 | 80 | NM_001166169.1 | 79 |
| NEK6 | 10783 | NP_001159642.1 | 82 | NM_001166170.1 | 81 |
| NEK6 | 10783 | NP_001159643.1 | 84 | NM_001166171.1 | 83 |
| NEK6 | 10783 | NP_055212.2 | 86 | NM_014397.5 | 85 |
| AQP11 | 282679 | NP_766627.1 | 88 | NM_173039.2 | 87 |
| CCDC126 | 90693 | NP_620126.2 | 90 | NM_138771.3 | 89 |
| KLF6 | 1316 | NP_001153596.1 | 92 | NM_001160124.1 | 91 |
| KLF6 | 1316 | NP_001153597.1 | 94 | NM_001160125.1 | 93 |
| KLF6 | 1316 | NP_001291.3 | 96 | NM_001300.5 | 95 |
| ACPP | 55 | NP_001127666.1 | 100 | NM_001134194.1 | 99 |
| ACPP | 55 | NP_001090.2 | 98 | NM_001099.4 | 97 |
| IGFBP4 | 3487 | NP_001543.2 | 102 | NM_001552.2 | 101 |
| IGF1 | 3479 | NP_000609.1 | 104 | NM_000618.3 | 103 |
| IGF1 | 3479 | NP_001104753.1 | 106 | NM_001111283.1 | 105 |
| IGF1 | 3479 | NP_001104754.1 | 108 | NM_001111284.1 | 107 |
| IGF1 | 3479 | NP_001104755.1 | 110 | NM_001111285.1 | 109 |
| IRS1 | 3667 | NP_005535.1 | 112 | NM_005544.2 | 111 |
| FOSL2 | 2355 | NP_005244.1 | 114 | NM_005253.3 | 113 |
| HRAS | 3265 | NP_001123914.1 | 116 | NM_001130442.1 | 115 |
| HRAS | 3265 | NP_005334.1 | 118 | NM_005343.2 | 117 |
| HRAS | 3265 | NP_789765.1 | 120 | NM_176795.3 | 119 |
| CLU | 1191 | NP_001164609.1 | 122 | NM_001171138.1 | 121 |
| CLU | 1191 | NP_001822.2 | 124 | NM_001831.2 | 123 |
| CLU | 1191 | NP_976084.1 | 126 | NM_203339.1 | 125 |
| HSD17B1 | 3292 | NP_000404.2 | 128 | NM_000413.2 | 127 |
| HSDL1 | 83693 | NP_001139523.1 | 130 | NM_001146051.1 | 129 |
| HSDL1 | 83693 | NP_113651.4 | 132 | NM_031463.4 | 131 |
| HSD11B2 | 3291 | NP_000187.3 | 134 | NM_000196.3 | 133 |
| STC1 | 6781 | NP_003146.1 | 136 | NM_003155.2 | 135 |
| CYP11A1 | 1583 | NP_000772.2 | 138 | NM_000781.2 | 137 |
| CYP11A1 | 1583 | NP_001093243.1 | 140 | NM_001099773.1 | 139 |
| GMNN | 51053 | NP_056979.1 | 142 | NM_015895.3 | 141 |
| HSD3B2 | 3284 | NP_000189.1 | 144 | NM_000198.3 | 143 |
| HSD3B2 | 3284 | NP_001159592.1 | 146 | NM_001166120.1 | 145 |
| CYP19A1 | 1588 | NP_000094.2 | 148 | NM_000103.3 | 147 |
| CYP19A1 | 1588 | NP_112503.1 | 150 | NM_031226.2 | 149 |
| IGFBP5 | 3488 | NP_000590.1 | 152 | NM_000599.3 | 151 |
| KCNK3 | 3777 | NP_002237.1 | 154 | NM_002246.2 | 153 |
| SMAD7 | 4092 | NP_001177750.1 | 156 | NM_001190821.1 | 155 |
| SMAD7 | 4092 | NP_001177751.1 | 158 | NM_001190822.1 | 157 |
| SMAD7 | 4092 | NP_001177752.1 | 160 | NM_001190823.1 | 159 |
| SMAD7 | 4092 | NP_005895.1 | 162 | NM_005904.3 | 161 |
| EGR3 | 1960 | NP_001186809.1 | 164 | NM_001199880.1 | 163 |
| EGR3 | 1960 | NP_001186810.1 | 166 | NM_001199881.1 | 165 |
| EGR3 | 1960 | NP_004421.2 | 168 | NM_004430.2 | 167 |
| FN1 | 2335 | NP_002017.1 | 170 | NM_002026.2 | 169 |
| FN1 | 2335 | NP_473375.2 | 172 | NM_054034.2 | 171 |
| FN1 | 2335 | NP_997639.1 | 174 | NM_212474.1 | 173 |
| FN1 | 2335 | NP_997641.1 | 176 | NM_212476.1 | 175 |
| FN1 | 2335 | NP_997643.1 | 178 | NM_212478.1 | 177 |
| FN1 | 2335 | NP_997647.1 | 180 | NM_212482.1 | 179 |

TABLE 4
Cattle (Bos Taurus) Marker Genes

| Gene | Gene ID | Protein ID | SEQ ID NO. | mRNA ID | SEQ ID NO. |
|---|---|---|---|---|---|
| NEK6 | 515816 | NP_001092458.1 | 182 | NM_001098988.1 | 181 |
| AQP11 | 510038 | NP_001103539.1 | 184 | NM_001110069.1 | 183 |
| CCDC126 | 782956 | NP_001075941.1 | 186 | NM_001082472.2 | 185 |
| KLF6 | 505884 | NP_001030348.2 | 188 | NM_001035271.2 | 187 |
| ACPP | 504700 | NP_001092336.1 | 190 | NM_001098866.1 | 189 |
| IGFBP4 | 282262 | NP_776982.1 | 192 | NM_174557.3 | 191 |
| IGF1 | 281239 | NP_001071296.1 | 194 | NM_001077828.1 | 193 |
| IRS1 | 538598 | XP_581382.2 | 196 | XM_581382.3 | 195 |
| IRS1 | 538598 | XP_002685688.1 | 198 | XM_002685642.1 | 197 |
| FOSL2 | 509889 | NP_001179879.1 | 200 | NM_001192950.1 | 199 |
| FOSL2 | 509889 | XP_002691497.1 | 202 | XM_002691451.1 | 201 |
| HRAS | 513012 | XP_590626.2 | 204 | XM_590626.2 | 203 |
| HRAS | 513012 | XP_879748.1 | 206 | XM_874655.1 | 205 |
| HRAS | 513012 | XP_879663.1 | 208 | XM_874570.1 | 207 |
| CLU | 280750 | NP_776327.1 | 210 | NM_173902.2 | 209 |
| HSD17B1 | 785989 | XP_001253408.1 | 212 | XM_001253407.1 | 211 |
| HSD17B1 | 353107 | NP_001095835.1 | 214 | NM_001102365.1 | 213 |
| HSDL1 | 505213 | NP_001092341.1 | 216 | NM_001098871.1 | 215 |
| HSD11B2 | 282434 | NP_777067.1 | 218 | NM_174642.1 | 217 |
| STC1 | 338078 | NP_788842.2 | 220 | NM_176669.3 | 219 |
| CYP11A1 | 338048 | NP_788817.1 | 222 | NM_176644.2 | 221 |
| GMNN | 526377 | NP_001020508.1 | 224 | NM_001025337.1 | 223 |
| CYP19A1 | 281740 | NP_776730.1 | 226 | NM_174305.1 | 225 |
| IGFBP5 | 404185 | NP_001098797.1 | 228 | NM_001105327.1 | 227 |
| KCNK3 | 519188 | XP_597401.4 | 230 | XM_597401.5 | 229 |
| KCNK3 | 519188 | XP_002691504.1 | 232 | XM_002691458.1 | 231 |
| SMAD7 | 535916 | NP_001179794.1 | 234 | NM_001192865.1 | 233 |
| SMAD7 | 535916 | XP_002697809.1 | 236 | XM_002697763.1 | 235 |
| SMAD7 | 535916 | XP_616030.3 | 238 | XM_616030.3 | 237 |
| EGR3 | 526233 | XP_604596.4 | 240 | XM_604596.5 | 239 |
| EGR3 | 526233 | XP_002689819.1 | 242 | XM_002689773.1 | 241 |
| FN1 | 280794 | NP_001157250.1 | 244 | NM_001163778.1 | 243 |

TABLE 5
Mouse (Mus Musculus) Marker Genes

| Gene | Gene ID | Protein ID | mRNA ID |
|---|---|---|---|
| NEK6 | 59126 | NP_001153103.1 | NM_001159631.1 |
| NEK6 | 59126 | NP_067619.1 | NM_021606.3 |
| AQP11 | 66333 | NP_780314.1 | NM_175105.3 |
| CCDC126 | 57895 | NP_780307.1 | NM_175098.5 |
| KLF6 | 23849 | NP_035933.2 | NM_011803.2 |
| ACPP | 56318 | NP_062781.2 | NM_019807.2 |
| ACPP | 56318 | NP_997551.1 | NM_207668.2 |
| IGFBP4 | 16010 | NP_034647.1 | NM_010517.3 |
| IGF1 | 16000 | NP_001104744.1 | NM_001111274.1 |
| IGF1 | 16000 | NP_001104745.1 | NM_001111275.1 |
| IGF1 | 16000 | NP_001104745.1 | NM_001111275.1 |
| IGF1 | 16000 | NP_034642.2 | NM_010512.4 |
| IGF1 | 16000 | NP_908941.1 | NM_184052.3 |
| IRS1 | 16367 | NP_034700.2 | NM_010570.4 |
| FOSL2 | 14284 | NP_032063.2 | NM_008037.4 |
| HRAS | 15461 | NP_001123915.1 | NM_001130443.1 |
| HRAS | 15461 | NP_001123916.1 | NM_001130444.1 |
| HRAS | 15461 | NP_032310.2 | NM_008284.2 |
| CLU | 12759 | NP_038520.2 | NM_013492.2 |
| HSD17B1 | 15485 | NP_034605.1 | NM_010475.1 |
| HSDL1 | 72552 | NP_780394.1 | NM_175185.4 |
| HSD11B2 | 15484 | NP_032315.2 | NM_008289.2 |
| STC1 | 20855 | NP_033311.3 | NM_009285.3 |
| CYP11A1 | 13070 | NP_062753.3 | NM_019779.3 |
| GMNN | 57441 | NP_065592.1 | NM_020567.2 |
| HSD3B2 | 15493 | NP_694873.1 | NM_153193.3 |
| HSD3B1 | 15492 | NP_032319.1 | NM_008293.3 |
| CYP19A1 | 13075 | NP_031836.1 | NM_007810.3 |
| IGFBP5 | 16011 | NP_034648.2 | NM_010518.2 |
| KCNK3 | 16527 | NP_034738.1 | NM_010608.2 |
| SMAD7 | 17131 | NP_001036125.1 | NM_001042660.1 |
| EGR3 | 13655 | NP_061251.1 | NM_018781.2 |
| FN1 | 14268 | NP_034363.1 | NM_010233.1 |

TABLE 6
Rat (Rattus Norvegicus) Marker Genes

| Gene | Gene ID | Protein ID | mRNA ID |
|---|---|---|---|
| NEK6 | 360161 | NP_891998.1 | NM_182953.1 |
| AQP11 | 286758 | NP_775128.1 | NM_173105.1 |
| CCDC126 | 500117 | NP_001102702.1 | NM_001109232.2 |
| KLF6 | 58954 | NP_113830.1 | NM_031642.2 |
| ACPP | 56780 | NP_001128373.1 | NM_001134901.1 |
| ACPP | 56780 | NP_064457.1 | NM_020072.1 |
| IGFBP4 | 360622 | NP_001004274.1 | NM_001004274.2 |
| IGF1 | 24482 | NP_001075947.1 | NM_001082478.1 |
| IGF1 | 24482 | NP_001075948.1 | NM_001082479.1 |
| IGF1 | 24482 | NP_001075946.2 | NM_001082477.2 |
| IGF1 | 24482 | NP_849197.3 | NM_178866.4 |
| IRS1 | 25467 | NP_037101.1 | NM_012969.1 |
| FOSL2 | 25446 | NP_037086.1 | NM_012954.1 |
| HRAS | 293621 | NP_001091711.1 | NM_001098241.1 |
| HRAS | 293621 | NP_001123913.1 | NM_001130441.1 |
| CLU | 24854 | NP_444180.2 | NM_053021.2 |
| HSD17B1 | 25322 | NP_036983.1 | NM_012851.1 |
| HSDL1 | 361418 | NP_001020067.1 | NM_001024896.1 |
| HSD11B2 | 25117 | NP_058777.1 | NM_017081.1 |
| STC1 | 81801 | NP_112385.1 | NM_031123.2 |
| CYP11A1 | 29680 | NP_058982.1 | NM_017286.2 |
| GMNN | 291137 | NP_001099582.1 | NM_001106112.1 |
| HSD3B2 | 682974 | NP_001036084.1 | NM_001042619.1 |
| HSD3B1 | 360348 | NP_001007720.3 | NM_001007719.3 |
| HSD3B6 | 29632 | NP_058961.4 | NM_017265.4 |
| CYP19A1 | 25147 | NP_058781.2 | NM_017085.2 |
| IGFBP5 | 25285 | NP_036949.1 | NM_012817.1 |
| KCNK3 | 29553 | NP_203694.1 | NM_033376.1 |
| SMAD7 | 81516 | NP_110485.1 | NM_030858.1 |
| EGR3 | 25148 | NP_058782.1 | NM_017086.1 |
| FN1 | 25661 | NP_062016.2 | NM_019143.2 |

TABLE 7
Selected Marker Genes from Pig (Sus Scrofa)

| Gene | Gene ID | Protein ID | SEQ ID NO. | mRNA ID | SEQ ID NO. |
|---|---|---|---|---|---|
| KLF6 | 100174961 | NP_001127825.1 | 246 | NM_001134353.2 | 245 |
| IGFBP4 | 100144490 | NP_001116601.1 | 248 | NM_001123129.1 | 247 |
| IGF1 | 397491 | NP_999421.1 | 250 | NM_214256.1 | 249 |
| HRAS | 733587 | NP_001038002.1 | 252 | NM_001044537.1 | 251 |
| CLU | 397025 | NP_999136.1 | 254 | NM_213971.1 | 253 |
| HSD17B1 | 100147712 | NP_001121944.1 | 256 | NM_001128472.1 | 255 |
| HSD11B2 | 396948 | NP_999078.1 | 258 | NM_213913.1 | 257 |
| STC1 | 100125345 | NP_001096682.1 | 260 | NM_001103212.1 | 259 |
| CYP11A1 | 403329 | NP_999592.1 | 262 | NM_214427.1 | 261 |
| CYP19A1 | 403331 | NP_999594.1 | 264 | NM_214429.1 | 263 |
| IGFBP5 | 397182 | NP_999264.1 | 266 | NM_214099.1 | 265 |
| SMAD7 | 100157769 | XP_001927617.1 | 268 | XM_001927582.1 | 267 |
| ACPP | 100302507 | XP_003132467.1 | 270 | XM_003132419.1 | 269 |
| IRS1 | 100294708 | ACG59405.1 | 272 | EU681268.1 | 271 |
| EGR3 | 100516296 | XP_003132855.1 | 274 | XM_003132807.1 | 273 |
| FN1 | 397620 | XP_003133689.1 | 276 | XM_003133641.1 | 275 |
| FN1 | 397620 | XP_003133690.1 | 278 | XM_003133642.1 | 277 |
| FN1 | 397620 | XP_003133691.1 | 280 | XM_003133643.1 | 279 |

TABLE 8
Selected Marker Genes from Sheep (Ovis Aries)

| Gene | Gene ID | Protein ID | mRNA ID |
|---|---|---|---|
| IGFBP4 | 443470 | NP_001127774.1 | NM_001134302.1 |
| IGF1 | 443318 | NP_001009774.1 | NM_001009774.2 |
| HSD11B2 | 443530 | NP_001009460.1 | NM_001009460.1 |
| CYP11A1 | 100048994 | NP_001087258.1 | NM_001093789.1 |
| CYP19A1 | 100144423 | NP_001116472.1 | NM_001123000.1 |
| IGFBP5 | 443133 | NP_001123205.1 | NM_001129733.1 |
| KCNK3 | 100141302 | NP_001186719.1 | NM_001199790.1 |
| SMAD7 | 443160 | AAL30899.1 | AF436855.1 |
| FN1 | 100216462 | ACJ24822.1 | FJ234417.1 |
| STC1 | 100294584 | UNREPORTED | UNREPORTED |

The practice of the invention is readily adapted to kit form. The identification of the 24 markers and the determination of the association between change in marker gene expression in cumulus cells and attendant oocyte quality provides the basis for clinical diagnostic kits based on either mRNA or protein expression.

Basic materials and reagents required for determination of oocyte quality according to the invention may be assembled in a kit. In certain embodiments, the kit comprises at least one reagent that specifically detects expression levels of at least one gene selected from the 24 marker genes disclosed herein, and instructions for using the kit according to one or more methods of the invention. Each kit necessarily comprises reagents which render the procedure specific. Thus, for detecting mRNA expressed by at least one marker gene of the set, the reagent will comprise a nucleic acid probe complementary to mRNA, such as, for example, a cDNA or an oligonucleotide. The nucleic acid probe may or may not be immobilized on a substrate surface (e.g., a microarray). For detecting a polypeptide product encoded by at least one gene of the set, the reagent will comprise an antibody that specifically binds to the polypeptide.

Depending on the procedure, the kit may further comprise one or more of: extraction buffer and/or reagents, amplification buffer and/or reagents, hybridization buffer and/or reagents, immunodetection buffer and/or reagents, labeling buffer and/or reagents, and detection means. Protocols for using these buffers and reagents for performing different steps of the procedure may also be included in the kit.

Reagents may be supplied in a solid (e.g., lyophilized) or liquid form. Kits of the present invention may optionally comprise different containers (e.g., vial, ampoule, test tube, flask or bottle) for each individual buffer and/or reagent. Each component will generally be suitable as aliquoted in its respective container or provided in a concentrated form. Other containers suitable for conducting certain steps for the disclosed methods may also be provided. In certain embodiments, the kits of the present invention further comprise control samples.

Instructions for using the kit according to one or more methods of the invention may comprise instructions for processing the cumulus cell or granulosa cells samples, and/or performing the test, and instructions for interpreting the results.

In one embodiment, a kit for determining mRNA expression of marker genes by quantitative RT-PCR would include standard primers and other reagents for performing quantitative RT-PCR, with reaction components formulated for optimized success in detection for each gene to be assayed. The amplification primers may be selected based on the nucleotide sequence of the relevant marker gene(s), depending on the species of mammalian oocyte being assessed. Any single or combination of the 24 markers genes could be incorporated into the kit. To provide users options to choose balance between coverage and cost, different kits could provide different collections of primers for the marker genes targeted for analysis.

Also a kit for determining mRNA expression of marker genes would advantageously further contain primers for a suitable constitutively expressed mRNA to serve as an internal standard (e.g., histone H2A or mitochondrial ribosomal protein S18C gene). A kit may further optionally contain a control cDNA mixture to serve as a positive control, a control cDNA library for confirming detection and signal intensities within acceptable parameters. A kit may yet further optionally contain control cDNA libraries representing cumulus cells from high and low quality oocytes, or synthetic mixtures and amounts of marker and control cDNAs that mimic the molar amounts of targets in such libraries.

The molecular kit would include instructional material that informs the user of relationship between expression level and oocyte quality, such as a package insert comprising the odds ratios and/or % increase in oocyte quality per ΔCt unit of Table 1. The instructional material may comprise a publication, a recording, a diagram, or any other medium of expression which can be used to communicate the usefulness of the method of the invention in the kit for assessment of oocyte quality. The package insert may comprise text housed in any physical medium, e.g., paper, cardboard, film, or may be housed in an electronic medium such as a diskette, chip, memory stick or other electronic storage form. The instructional material of the kit of the invention may, for example, be affixed to a container which contains other contents of the kit, or be shipped together with a container which contains the kit. Alternatively, the instructional material may be shipped separately from the container with the intention that the instructional material and the contents of the kit be used cooperatively by the recipient.

A compatible qRT-PCR instrumentation platform may be provided, equipped with a user interface in which the sample information is input for each run (including controls) and the output is a report of CT values, normalized CT values, and relative change in oocyte quality from controls or between oocytes in a population from an individual patient based on automated calculations using marker expression values and odds ratios for each marker assayed.

In another embodiment, a kit for determining expressed protein levels of marker genes is provided. Such kits can comprise an appropriate set of reagents that bind to the marker proteins of the invention. For example, for each maker protein, the kit can comprise an antibody, an antibody derivative, or an antibody fragment that binds specifically with the marker protein.

For antibody-based kits, the kit can comprise, for example: (1) a first antibody (e.g., attached to a solid support) that binds to a marker protein; and, optionally, (2) a second, different antibody that binds to either the protein or the first antibody and is conjugated to a detectable label. The kit can further comprise components necessary for detecting the detectable label (e.g., an enzyme or a substrate), and instrumentation for detection and measurement. A kit may further optionally contain aliquots of known amounts of maker protein to serve as reference standards, or reference samples representing marker protein from cumulus cells of high and low quality oocytes.

The kit for determination of marker protein expression may further contain a package insert that informs the user of the relationship between expression level and oocyte quality, such as a package insert comprising the odds ratios and/or % increase in oocyte quality per ΔCt unit of Table 1.

In addition to the specific biomarker sequences identified herein by accession number and/or sequence, the invention also contemplates the detection in a test sample of variants that are at least 90%, at least 95%, at least 96%, at least 97%, at least 98%, or at least 99% identical to those exemplified biomarker sequences.

The determination of percent identity between two nucleotide or amino acid sequences (“sequence identity”) can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator “http://blast(dot)ncbi(dot)nlm(dot)nih(dot)gov/Blast(dot)cgi”. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAT) can be used.

The practice of the invention is illustrated by the following non-limiting example. The invention should not be construed to be limited solely to the compositions and methods described herein, but should be construed to include other compositions and methods as well. One of skill in the art will know that other compositions and methods are available to perform the procedures described herein.

### EXAMPLE

### Identification of Differentially Expressed Genes

### 1. Collection and Lysis of Rhesus Cumulus Cells

Adult female rhesus macaques were housed individually with a 0600-1800 light cycle and maintained at a temperature of 25-27° C. Animals were allowed to socialize by being housed in pairs during the day from approximately 0800 to 1400. Animals were fed a diet of Purina Monkey Chow and water ad libitum and seasonal produce, seeds, and cereal were offered as supplements for environmental enrichment. Only females with a history of normal menstrual cycles were selected for study.

Females were observed daily for signs of vaginal bleeding and the first day of menses was assigned cycle day 1. Beginning on cycle day 1-4 recombinant human FSH (r-hFSH: Organon, West Orange, N.J.) was administered (37.5 IU) twice daily, intramuscularly for 7 days total. For in vitro maturation (IVM) experiments, cumulus-oocyte complexes (COCs) were collected on day 8. To obtain in vivo matured (VVM) oocytes, females were given recombinant hCG (1,000 IU Ovidrel; Serono, Rockland, Mass.) on treatment day 8 in addition to the FSH treatment outlined above. COCs were aspirated from follicles at 28-30 hours following hCG by ultrasound-guided aspiration (VandeVoort and Tarantal, (1991) J Med Primatol 20:110-116; VandeVoort and Tarantal A F, (2001) J Med Primatol 30:304-307). Oocytes were retrieved from aspirates as described (VandeVoort, et al. (2003) Theriogenology 59:699-707).

All cumulus-oocyte complexes were collected into Tyrode lactate (TL)-HEPES medium (37° C.) containing 0.1 mg/ml polyvinyl alcohol and 5 ng/ml recombinant human FSH (r-hFSH; Organon). Aspirates were immediately placed in a heated isolette (37° C.), where cumulus-oocyte complexes were retrieved from aspirates and placed in fresh TL-HEPES medium. Immature oocytes were recovered at the germinal vesicle stage and cultured for 24 h in 70-μl drops under oil in CMRL medium (Boatman and Bavister (1984) J Reprod Fertil 71:357-366) containing 10% bovine calf serum (Gem Cell, Woodland, Calif.), hFSH, hLH (0.03 IU/ml Pergonal, Ares-Serono), and 1 μg/ml androstenedione (Steraloids, Newport, R.I.) (also reported as CMRLb medium (Schramm et al., (2003) Hum Reprod 18:826-833)). Cumulus cells were obtained from IVM oocytes immediately after collection or after culture for 24 hours, and from VVM oocytes only immediately after collection. The cumulus oocyte complexes were placed individually into drops of TL-HEPES medium and cumulus cells were removed from oocytes by trituration through a small bore pipette. Only cumulus cells from oocytes that had a germinal vesicle (GV) were included in the pre-maturation IVM control group and only oocytes with one polar body were utilized for the IVM and VVM groups.

### 2. RNA Isolation, Amplification and Array Hybridization

Total RNA was isolated from cumulus cells using the PicoPure RNA isolation kit (MDS Analytical Technologies, Sunnyvale, Calif.) according to the manufacturer's instruction. Fifty ng of total RNA from each sample were subjected to two rounds of cDNA synthesis and in-vitro transcription labeling to achieve a linear amplification (Eukaryotic Small Sample Target Labeling Assay, Affymetrix GeneChip Expression Analysis Technical Manual) with minor modifications (initial 5 μl volume for annealing and reverse transcription for 30 minutes at 42° C. followed by 30 minutes at 45° C.). The biotin-labeled cRNA samples were fragmented and 10 μg were hybridized onto Affymetrix Rhesus Genome GeneChip arrays. Post-hybridization washing, staining and scanning were performed as described in the Affymetrix GeneChip Expression Analysis Technical Manual.

### 3. Microarray Data Analysis

Transcriptome profiles were generated as follows from three different groups of rhesus monkey cumulus cells using the Affymetrix Rhesus Genome arrays.

A first cumulus cell group comprised pre-maturation cumulus cells (PM-CC) from cumulus-oocyte complexes (COCs) collected and lysed on day 8 immediately after collection from females that received seven days of FSH hormone injections. A second cumulus cell group (IVM-CC) was from COCs that were collected on day 8, followed by maturation of the intact COCs in vitro for 24 hours by exposure to gonadotropin levels that are known to result in oocyte maturation and luteinization of granulosa cells (de Prada and VandeVoort, (2008) J Assist Reprod Genet 25:145-158; Chaffin et al., (2003) Endocrinology 144:1249-1256). On day 9, cumulus cells were separated from oocytes and lysed. A third group comprised in vivo matured cumulus cells (VVM-CC) obtained from COCs on day 9 from rhesus monkeys that received the seven-day FSH regimen and then an hCG dose on day 8. Thus, the only difference between the IVM and VVM cells was whether exposure to gonadotropins to stimulate final oocyte maturation occurred in vitro or in vivo during the period from day 8 to day 9.

Inspecting array data, all quality control parameters were within the acceptable ranges for all samples (data not shown). The average detection rate for PM-CC samples was 47%, corresponding to approximately 25,000 probe sets. The detection rates for IVM-CC and VVM-CC were 38% and 41%, respectively. A hierarchical clustering analysis (HCL) revealed that the biological replicates cluster to their corresponding groups without any apparent outliers (data not shown). The three-dimensional Principal Component Analysis (PCA) also showed the clustering of all replicates to their corresponding groups (data not shown).

Probe hybridization intensity data were imported into the Affymetrix Expression Console Software and summarized using the Robust Multichip Analysis (RMA) algorithm with a global background correction and a quantile normalization (Irizarry et al., (2003) Nucleic Acids Res 31:e15). To simplify and focus analysis, 1,099 genes with inconsistencies between probes sets were omitted from further consideration, and only the genes that differed in expression at the threshold of 2-fold or greater were employed for Interpretative Phenomenological Analysis (IPA). In order to minimize false detections, expression data were filtered based on Present/Absent calls determined by MAS 5.0 algorithm (Affymetrix) at the default settings (detection p-value <0.05 and tau=0.015).

Only those probe sets called ‘Present’ in all biological replicates of one of the two groups in comparison were selected for the analysis. To minimize the chance of false positives in subsequent analyses, probe sets with maximum raw intensity values <100 were removed omitted from the data set. To identify differentially expressed genes, the Significance Analysis of Microarray (SAM; Tusher et al., (2001) Proc Natl Acad Sci USA 98:5116-5121) was performed. Differentially expressed probe sets were identified at the false discovery rate (FDR) lower than 1% and the Student's t-test was used to select further for the genes with statistical significance (P<0.01).

The initial comparisons between cumulus cells obtained following in vivo or in vitro maturation of cumulus oocyte complexes revealed more than 500 mRNAs differentially regulated in cumulus cells comparing in vitro maturation (IVM) and in vivo maturation (VVM) using array data (data not shown). Based on knowledge about specific biological pathways related to the affected genes, additional mRNAs were evaluated and found to be differentially expressed as well. The 43 genes in Table 9 were then selected as candidates for markers to distinguish between cumulus cell phenotypes associated with different oocyte quality. These 43 genes were chosen based on a combination of fold change, array hybridization signal strength and biological function.

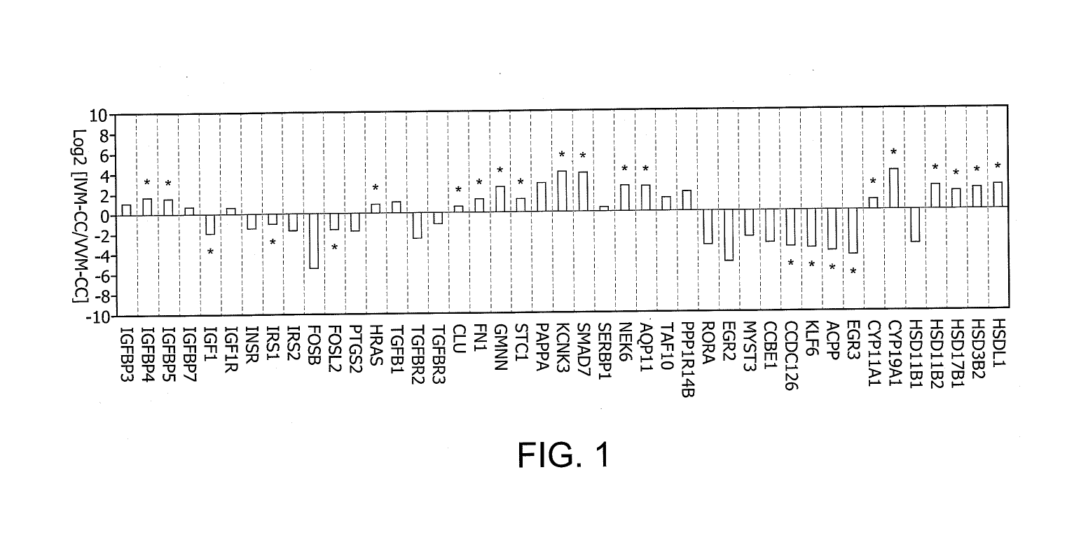

The results of the microarray data analysis for the 43 genes of Table 9 are set forth in FIG. 1, showing differences in gene expression in cumulus cells comparing in vitro and in vivo maturation (IVM and VVM) based on normalized array average raw intensity values. The ratio of expression (log2) is shown.

TABLE 9
Candidate Markers for Oocyte Quality

| | | | | |
|---|---|---|---|---|
| ACPP | AQP11 | CCBE1 | CCDC126 | CLU |
| CYP11A1 | CYP19A1 | EGR2 | EGR3 | FN1 |
| FOSB | FOSL2 | GMNN | HRAS | HSD17B1 |
| HSD11B2 | HSD3B2 | HSDL1 | IGFBP3 | IGFBP4 |
| IGFBP5 | IGFBP7 | IGF1 | IGF1R | INSR |
| IRS1¹ | IRS2 | KCNK3 | KLF6 | MYST3 |
| NEK6 | PAPPA | PPP1R14B | PTGS2 | RORA |
| SMAD7 | SERBP1 | SD11B2 | STC1 | TAF 10 |
| TGFB1 | TGFBR2 | TGFBR3. | | |

¹Based on the homologous rheses gene IRS4

4. Real Time qRT-PCR Analysis

The expression of individual mRNAs from the 43 genes in Table 9 was tested further using a quantitative real time RT-PCR assay. Each sample used for the assay represented a pool of cumulus cells obtained from cumulus-oocyte complexes of a single rhesus monkey. Nine samples of VVM and four samples of IVM cumulus cells were processed, representing nine female monkeys that had undergone stimulation for VVM and four females that had undergone stimulation for IVM. Total RNA was isolated using the PicoPure RNA isolation kit (Molecular Devices, Sunnyvale, Calif.), and subjected to a whole transcriptome amplification using the QuantiTect whole transcriptome kit (Qiagen, Valencia, Calif.). Custom TaqMan gene expression assays were designed based on rhesus cDNA sequences using Primer Express software v3.0 (Applied Biosystems, Foster City, Calif.). Quantitative real time PCR was performed on approximately 100 ng of cDNA for each sample with ABI StepOne Plus instrument according to the manufacturer's recommendations (Applied Biosystems, Foster City, Calif.). Primer sequences employed for the assays are given in Table 10 (forward) and Table 11 (reverse). Reporter sequences are provided in Table 12. The mRNA abundance of each target gene was normalized to the endogenous mitochondrial ribosomal protein S18C gene (MRPS18C) for sample-to-sample comparisons. The relative expression ratio of IVM to VVM groups was obtained by the comparative CT method (Livak and Schmittgen, (20010) Methods 25:402-408).

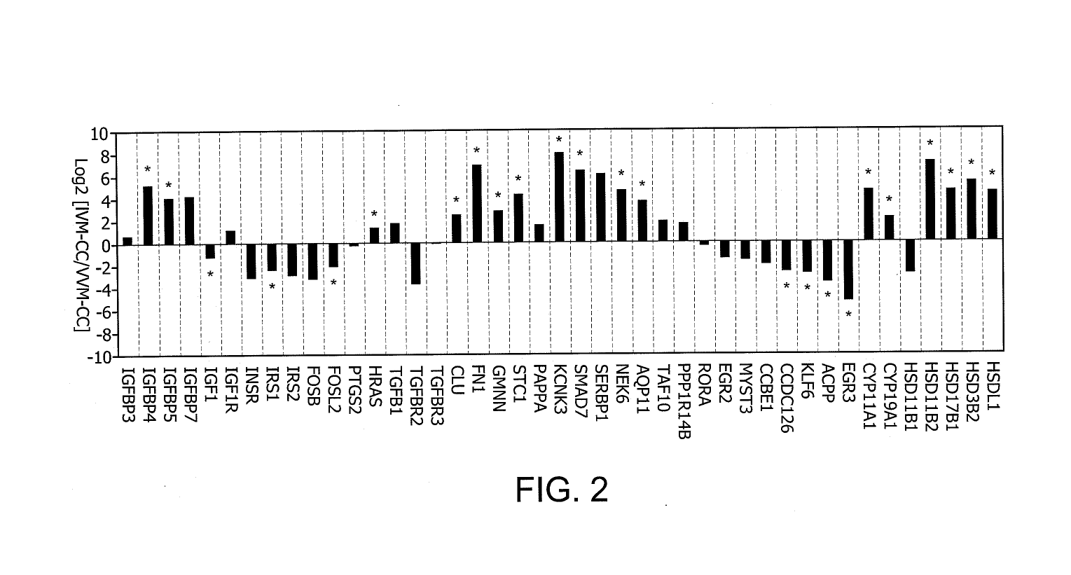

The results of the quantitative real time RT-PCR are set forth in FIG. 2, showing the differences in gene expression in cumulus cells comparing in vitro and in vivo maturation (IVM and VVM). The ratio of expression (log2) after normalization to the internal standard is shown.

TABLE 10
qRT-PCR Forward Primer Sequences

| Genes | Forward Primer Seq. | SEQ ID NO: |
|---|---|---|
| NEK6 | GGAAGGATGGGATGTAAAAAAGCT | 1 |
| AQP11 | GGAAGCCGCCTTATAGTTTTCA | 2 |
| CCDC126 | CCAAATCTCTGTTCTCTTCTGTGTACTC | 3 |
| KLF6 | TGGCTGGGCTGGTATTTTGT | 4 |
| ACPP | AAGCCACCCCCGTTCCT | 5 |
| EGR3 | AAAGCAACAAAAGAAAATGCACTCT | 6 |
| IGFBP4 | TGGACTGAATGTGCCTAATGGA | 7 |
| IGF1 | GCAGCGCCGCATCAG | 8 |
| IRS4¹ | TTGGCAATTGAATGGAAGCA | 9 |
| FOSL2 | CCCAAGACCTGGCGTGAT | 10 |
| HRAS | GGAAGCTGAACCCTCCTGATG | 11 |
| CLU | GGTGTCCCAGCTGGCAAA | 12 |
| FN1 | GCCTGTTCTGCTTCAAAGTATTCA | 13 |
| HSD17B1 | GGGCTACCCTTCAATGACGTT | 14 |
| HSDL1 | GGAGGCTGAGGCAGGAGAAT | 15 |
| HSD11B2 | AGGCCAAGGTTTCCCAGTGT | 16 |
| STC1 | AGTTCTTTACTCGTCCCCTTTCATC | 17 |
| CYP11A1 | GGCACCACATTCAACCTCATC | 18 |
| GMNN | TGTTGAGAATTTTACTGCCGAAGT | 19 |
| HSD3B2 | TGGCCCAACCAGAAGCTTT | 20 |
| CYP19A1 | CTCTTAAAGAATGTTTTGGTCTCCATT | 21 |
| IGFBP5 | GGGTGAACAATTTTGTGGCTATTT | 22 |
| KCNK3 | TGTGCTGGCCACTGATTCC | 23 |
| SMAD7 | CAAGAAGGATTTGGTCCGTCATA | 24 |

¹IRS4 is the rhesus homolog of the human gene IRS1.

TABLE 11
qRT-PCR Reverse Primer Sequences

| Gene | Reverse Primer Seq. | SEQ ID NO. |
|---|---|---|
| NEK6 | CGCTAGGAAGTTGCAGAACCA | 25 |
| AQP11 | GGATGCCTCATTTTCACAGTAATTT | 26 |
| CCDC126 | AACTTCTACCTGGCAATGGCATA | 27 |
| KLF6 | AAACCAGTTATGTGAGCGTTAGTCA | 28 |
| ACPP | AACACAAAGAAGCGTATCAATCGA | 29 |
| EGR3 | CCCCATAGGTTTTCCTGTTTAAAA | 30 |
| IGFBP4 | GACCCAGGAAGCCCCTCAT | 31 |
| IGF1 | GTATGTAGGGTGGGTGTTGAGAGA | 32 |
| IRS4¹ | GGAATCACCCATCTCCTTCTCTTT | 33 |
| FOSL2 | GAGACAGCTGCTCATCTCTCCTT | 34 |
| HRAS | CGTCAGGAGAGCACACACTTG | 35 |
| CLU | CGTGGTGACCCGCAGATAG | 36 |
| FN1 | CACCAAATCACAAGTTAGAATCACTTC | 37 |
| HSD17B1 | GACTCTCGCATAAGCCTTCGA | 38 |
| HSDL1 | GCACGATCTTGGCTCACTTCA | 39 |
| HSD11B2 | GGGTCTGTTTGGGCTCATGA | 40 |
| STC1 | GAAAGTCTCCCACCCCATCA | 41 |
| CYP11A1 | AAGGGCCAGAAGGTGAAGGA | 42 |
| GMNN | CCAGAATTGGCATTATGTAGTTACGT | 43 |
| HSD3B2 | TTGGGTAACAGCAAATCATATTGTCT | 44 |
| CYP19A1 | GCTTGTGAATTTTTCTTTGTGTACATGTAT | 45 |
| IGFBP5 | GGCCGGAGAAACCCTCAA | 46 |
| KCNK3 | CTCCTGCCCTGGTGACCTAA | 47 |
| SMAD7 | TAGAAGAAATGAAAGAAGAGTTAGGTGTCA | 48 |

¹IRS4 is the rhesus homolog of the human gene IRS1.

TABLE 12
qRT-PCR Reporter Sequences

| Genes | Reporter Sequence | SEQ ID NO. |
|---|---|---|
| NEK6 | AGGGCTATCCTTTACAAAT | 49 |
| AQP11 | CACTGGGACTTAAACAA | 50 |
| CCDC126 | CTACCTTTATGTGAAGAAAT | 51 |
| KLF6 | TCTTCCTGGAAATGAGCA | 52 |
| ACPP | ACATGACTGACAAAGAC | 53 |
| EGR3 | CTTATGTGAACTGAGAGAAA | 54 |
| IGFBP4 | AAGACCCACGTGCTAGG | 55 |
| IGF1 | ATCCACTCTTCTAGGGATAT | 56 |
| IRS4¹ | TAAGAAGAGGAAATCAAAGTC | 57 |
| FOSL2 | ACCATTGGCACCACCGT | 58 |
| HRAS | CCCCGGCTGCATGA | 59 |
| CLU | CGCAAGGCGAAGAC | 60 |
| FN1 | ACCGCTCAGTATTTTA | 61 |
| HSD17B1 | CCAGCAAGTTCGCG | 62 |
| HSDL1 | AGCCCGGGATGCAG | 63 |
| HSD11B2 | CTGCGCCTCTCCACT | 64 |
| STC1 | CTGGTACTCTGGCAAAT | 65 |
| CYP11A1 | TGCCTGAAAAGCC | 66 |
| GMNN | ACCTCCACTAGTTCTTT | 67 |
| HSD3B2 | TGTCCTAATCATACGCCAGAG | 68 |
| CYP19A1 | TAGTAGTCTGTGCATAAGGT | 69 |
| IGFBP5 | CCCTATAATTCTGACCCGCT | 70 |
| KCNK3 | TTGAGTCTCACAACAGCCTA | 71 |
| SMAD7 | CCAAGGTACCATCTCTAGG | 72 |

¹IRS4 is the rhesus homolog of the human gene IRS1.

5. qRT-PCR Analysis

Mean gene expression values were compared between groups (IVM vs. VVM) adjusted for background using analysis of covariance on rank-transformed expression levels. Pearson correlation coefficients were calculated to evaluate the degree of collinearity and similarity among the expression levels of the various genes. Gene expression levels were analyzed for a relationship to oocyte quality. A single gene generalized estimating equation (GEE) was used to calculate the relationship of gene expression to oocyte quality. GEE is a special case of the generalized linear model taking into consideration the occurrence of multiple oocytes per animal in the same analysis.

Odds ratios with 95% confidence intervals and p-values were calculated for each gene as each was related to oocyte quality. Univariate generalized estimating equations analysis (GEE) showed that the expression levels of 24 genes (marked with an asterisk in FIG. 2) were significantly related to oocyte quality (IVM vs. VVM), as shown in Table 13:

TABLE 13
Calculation of Oocyte Quality Odd Ratios

| | | | | | | | | % increase |
|---|---|---|---|---|---|---|---|---|
| | | | | | | | | in oocyte |
| | Paramet | 95% Confidence | | | Odds | | | quality per |
| Gene | Estimate | Limits | | P-value | ratio | (95% C.I.) | | ΔCt unit |
| NEK6 | 0.1761 | 0.0549 | 0.2973 | 0.0044 | 1.1926 | 1.0564 | 1.3462 | 19.26 |
| AQP11 | 0.4877 | 0.1308 | 0.8446 | 0.0074 | 1.6286 | 1.1397 | 2.3270 | 62.86 |
| CCDC126 | −0.1489 | −0.2730 | −0.0248 | 0.0187 | 0.8617 | 0.7611 | 0.9755 | −13.83 |
| KLF6 | −0.2233 | −0.4347 | −0.0119 | 0.0385 | 0.7999 | 0.6475 | 0.9882 | −20.01 |
| ACPP | −1.8279 | −3.4751 | −0.1807 | 0.0296 | 0.1608 | 0.0310 | 0.8347 | −83.92 |
| EGR3 | −0.0934 | −0.1851 | −0.0018 | 0.0458 | 0.9108 | 0.8310 | 0.9982 | −8.92 |
| IGFBP4 | 0.729 | 0.0135 | 1.4445 | 0.0458 | 2.0730 | 1.0136 | 4.2397 | 107.30 |
| IGF1 | −0.5011 | −0.9215 | −0.0807 | 0.0195 | 0.6059 | 0.3979 | 0.9225 | −39.41 |
| IRS1¹ | −1.0668 | −1.5369 | −0.5967 | <.0001 | 0.3441 | 0.2150 | 0.5506 | −65.59 |
| FOSL2 | −0.0406 | −0.0682 | −0.013 | 0.0039 | 0.9602 | 0.9341 | 0.9871 | −3.98 |
| HRAS | 0.1602 | 0.0187 | 0.3017 | 0.0265 | 1.1737 | 1.0189 | 1.3522 | 17.37 |
| CLU | 0.5684 | 0.4395 | 0.6973 | <.0001 | 1.7654 | 1.5519 | 2.0083 | 76.54 |
| FN1 | 0.097 | 0.0790 | 0.115 | <.0001 | 1.1019 | 1.0822 | 1.1219 | 10.19 |
| HSD17B1 | 0.9201 | 0.5145 | 1.3257 | <.0001 | 2.5095 | 1.6728 | 3.7648 | 150.95 |
| HSDL1 | 0.1361 | 0.1140 | 0.1582 | <.0001 | 1.1458 | 1.1208 | 1.1714 | 14.58 |
| HSD11B2 | 0.0873 | 0.0694 | 0.1051 | <.0001 | 1.0912 | 1.0719 | 1.1108 | 9.12 |
| STC1 | 0.7103 | 0.1056 | 1.315 | 0.0213 | 2.0346 | 1.1114 | 3.7248 | 103.46 |
| CYP11A1 | 0.1552 | 0.1287 | 0.1817 | <.0001 | 1.1679 | 1.1373 | 1.1993 | 16.79 |
| GMNN | 0.1466 | 0.1022 | 0.191 | <.0001 | 1.1579 | 1.1076 | 1.2105 | 15.79 |
| HSD3B2 | 0.648 | 0.1304 | 1.1656 | 0.0141 | 1.9117 | 1.1393 | 3.2078 | 91.17 |
| CYP19A1 | 2.4178 | 1.3035 | 3.5321 | <.0001 | 11.2211 | 3.6822 | 34.1957 | 1022.11 |
| IGFBP5 | 0.0283 | 0.0033 | 0.0533 | 0.0265 | 1.0287 | 1.0033 | 1.0547 | 2.87 |
| KCNK3 | 0.0002 | 0.0000 | 0.0003 | 0.0152 | 1.0002 | 1.0000 | 1.0003 | 0.02 |
| SMAD7 | 0.0324 | 0.0090 | 0.0557 | 0.0066 | 1.0329 | 1.0090 | 1.0573 | 3.29 |

¹Based on the homologous rhesus monkey gene IRS4.

The odds ratios calculated for each of these 24 genes allow quality of oocytes to be compared. For each one unit change in CT value, the odds of a quality oocyte increases or decreases as indicated by the odds ratio. This allows oocytes to be compared to each other or to a reference sample set in order to judge quality and select the oocytes based on probability of having desired developmental potential.

The disclosures of each and every patent, patent application, and publication cited herein are hereby incorporated herein by reference in their entirety.

One skilled in the art will readily appreciate that the present invention is well adapted to carry out the objects and obtain the ends and advantages mentioned, as well as those inherent therein. While the invention has been disclosed with reference to specific embodiments, it is apparent that other embodiments and variations of this invention may be devised by others skilled in the art without departing from the true spirit and scope used in the practice of the invention. The appended claims are intended to be construed to include all such embodiments and equivalent variations.

1. A method for evaluating the quality of a mammalian oocyte comprising:
(a) determining the level of expression of at least one marker gene of a set of marker genes comprising ACPP, AQP11, CCDC126, CLU, CYP11A1, CYP19A1, EGR3, FN1, FOSL2, GMNN, HRAS, HSD3B2, HSD17B1, HSD11B2, HSDL1, IGF1, IGFBP4, IGFBP5, IRS1, KCNK3, KLF6, NEK6, SMAD7 and STC1 in a sample derived from a cumulus cell or granulosa cell associated with the oocyte, after maturation of the oocyte;
(b) comparing the expression level of said at least one marker gene in the test sample with the expression level in a control, wherein detecting differential expression of the maker gene between the sample and the control is indicative of the quality of the oocyte.

2. The method according to claim 1, wherein the control is a sample derived from a cumulus cell or a granulosa cell associated with a mammalian oocyte of known quality.

3. The method according to claim 2 wherein the control is a sample derived from a cumulus cell or a granulosa cell associated with an in vitro matured mammalian oocyte.

4. The method according to claim 2 wherein the control sample is derived from a cumulus cell or a granulosa cell associated with an in vivo mammalian matured oocyte.

5. The method according to claim 2 wherein the level of expression of at least three of said marker genes is determined.

6. The method according to claim 5 wherein the marker genes comprise NEK6, AQP11 and IGF1.

7. The method according to claim 6 wherein the probability of the oocyte having a high quality (P) is given by the equation:
P=e^{5.608+0.645x+0.100y-2.17z}/1+e^{.}e^{5.608+0.645x+0.100y-2.17z}
wherein:
x is the expression level of NEK6 relative to the control;
y is the expression level of AQP 11 relative to the control; and
z is the expression level of IGF1 relative to the control.

8. The method according to claim 5 wherein the level of expression of at least ten of said marker genes is determined.

9. The method according to claim 2 wherein marker gene expression level is determined by determining the level of mRNA produced from said marker gene.

10. The method according to claim 9 wherein the level of mRNA is determined by reverse transcription polymerase chain reaction.

11. The method according to claim 9 wherein the mammalian oocyte is an oocyte of a domesticated mammal.

12. The method according to claim 11 wherein the domesticated mammal is a bovine, pig or sheep.

13. The method according to claim 12 wherein the domesticated mammal is a bovine.

14. The method according to claim 13 wherein the level of at least one of the following mRNAs is determined:
the NEK6 mRNA having Genbank Accession Number NM_{—}001098988.1 (SEQ ID NO: 181);
the AQP11 mRNA having Genbank Accession Number NM_{—}001110069.1 (SEQ ID NO: 183);
the CCDC 126 mRNA having Genbank Accession Number NM_{—}001082472.2 (SEQ ID NO: 185);
the KLF6 mRNA having Genbank Accession Number NM_{—}001035271.2 (SEQ ID NO: 187);
the ACPP mRNA having Genbank Accession Number NM_{—}001098866.1 (SEQ ID NO: 189);
the IGFBP4 mRNA having Genbank Accession Number NM_{—}174557.3 (SEQ ID NO: 191);
the IGF1 mRNA having Genbank Accession Number NM_{—}001077828.1 (SEQ ID NO: 193);
the IRS1 mRNA having Genbank Accession Number XM_{—}581382.3 (SEQ ID NO: 195);
the IRS1 mRNA having Genbank Accession Number XM_{—}002685642.1 (SEQ ID NO: 197);
the FOSL2 mRNA having Genbank Accession Number NM_{—}001192950.1(SEQ ID NO: 199);
the FOSL2 mRNA having Genbank Accession Number XM_{—}002691451.1(SEQ ID NO: 201);
the HRAS mRNA having Genbank Accession Number XM_{—}590626.2 (SEQ ID NO: 203);
the HRAS mRNA having Genbank Accession Number XM_{—}874655.1 (SEQ ID NO: 205);
the HRAS mRNA having Genbank Accession Number XM_{—}874570.1 (SEQ ID NO: 207);
the CLU mRNA having Genbank Accession Number NM_{—}173902.2 (SEQ ID NO: 209);
the HSD17B1 mRNA having Genbank Accession Number XM_{—}001253407.1 (SEQ ID NO: 211);
the HSD17B1 mRNA having Genbank Accession Number NM_{—}001102365.1 (SEQ ID NO: 213);
the HSDL1 mRNA having Genbank Accession Number NM_{—}001098871.1 (SEQ ID NO: 215);
the HSD11B2 mRNA having Genbank Accession Number NM_{—}174642.1 (SEQ ID NO: 217);
the STC1 mRNA having Genbank Accession Number NM_{—}176669.3 (SEQ ID NO: 219);
the CYP11A1 mRNA having Genbank Accession Number NM_{—}176644.2 (SEQ ID NO: 221);
the GMNN mRNA having Genbank Accession Number NM_{—}001025337.1 (SEQ ID NO: 223);
the CYP19A1 mRNA having Genbank Accession Number NM_{—}174305.1 (SEQ ID NO: 225);
the IGFBP5 mRNA having Genbank Accession Number NM_{—}001105327.1 (SEQ ID NO: 227);
the KCNK3 mRNA having Genbank Accession Number XM_{—}597401.5 (SEQ ID NO: 229);
the KCNK3 mRNA having Genbank Accession Number XM_{—}002691458.1 (SEQ ID NO: 231);
the SMAD7 mRNA having Genbank Accession Number NM_{—}001192865.1 (SEQ ID NO: 233);
the SMAD7 mRNA having Genbank Accession Number XM_{—}002697763.1 (SEQ ID NO: 235);
the SMAD7 mRNA having Genbank Accession Number XM_{—}616030.3 (SEQ ID NO: 237);
the EGR3 mRNA having Genbank Accession Number XM_{—}604596.5 (SEQ ID NO: 239);
the EGR3 mRNA having Genbank Accession Number XM_{—}002689773.1 (SEQ ID NO: 241); and
the FN1 mRNA having Genbank Accession Number NM_{—}001163778.1 (SEQ ID NO: 243).

15. The method according to claim 12 wherein the domesticated mammal is a pig.

16. The method according to claim 15 wherein the level of at least one of the following mRNAs is determined:
the KLF6 mRNA having Genbank Accession Number NM_{—}001134353.2 (SEQ ID NO: 245);
the IGFBP4 mRNA having Genbank Accession Number NM_{—}001123129.1 (SEQ ID NO: 247);
the IGF1 mRNA having Genbank Accession Number NM_{—}214256.1 (SEQ ID NO: 249);
the HRAS mRNA having Genbank Accession Number NM_{—}001044537.1 (SEQ ID NO: 251);
the CLU mRNA having Genbank Accession Number NM_{—}213971.1 (SEQ ID NO: 253);
the HSD17B1 mRNA having Genbank Accession Number NM_{—}001128472.1 (SEQ ID NO: 255);
the HSD11B2 mRNA having Genbank Accession Number NM_{—}213913.1 (SEQ ID NO: 257);
the STC1 mRNA having Genbank Accession Number NM_{—}001103212.1 (SEQ ID NO: 259);
the CYP11A1 mRNA having Genbank Accession Number NM_{—}214427.1 (SEQ ID NO: 261);
the CYP19A1 mRNA having Genbank Accession Number NM_{—}214429.1 (SEQ ID NO: 263);
the IGFBP5 mRNA having Genbank Accession Number NM_{—}214099.1 (SEQ ID NO: 265);
the SMAD7 mRNA having Genbank Accession Number XM_{—}001927582.1 (SEQ ID NO: 267);
the ACPP mRNA having Genbank Accession Number XM_{—}003132419.1 (SEQ ID NO: 269);
the IRS 1 mRNA having Genbank Accession Number EU681268.1 (SEQ ID NO: 271);
the EGR3 mRNA having Genbank Accession Number XM_{—}003132807.1 (SEQ ID NO: 273);
the FN1 mRNA having Genbank Accession Number XM_{—}003133641.1 (SEQ ID NO: 275);
the FN1 mRNA having Genbank Accession Number XM_{—}003133642.1 (SEQ ID NO: 277); and
the FN1 mRNA having Genbank Accession Number XM_{—}003133643.1 (SEQ ID NO: 279).

17. The method according to claim 9 wherein the mammalian oocyte is an oocyte of a human being.

18. The method according to claim 17 wherein the level of at least one of the following mRNAs is determined:
the NEK6 mRNA having Genbank Accession Number NM_{—}001145001.2 (SEQ ID NO: 73);
the NEK6 mRNA having Genbank Accession Number NM_{—}001166167.1 (SEQ ID NO: 75);
the NEK6 mRNA having Genbank Accession Number NM_{—}001166168.1 (SEQ ID NO: 77);
the NEK6 mRNA having Genbank Accession Number NM_{—}001166169.1 (SEQ ID NO: 79);
the NEK6 mRNA having Genbank Accession Number NM_{—}001166170.1 (SEQ ID NO: 81);
the NEK6 mRNA having Genbank Accession Number NM_{—}001166171.1 (SEQ ID NO: 83);
the NEK6 mRNA having Genbank Accession Number NM_{—}014397.5 (SEQ ID NO: 85);
the AQP11 mRNA having Genbank Accession Number NM_{—}173039.2 (SEQ ID NO: 87);
the CCDC126 mRNA having Genbank Accession Number NM_{—}138771.3 (SEQ ID NO: 89);
the KLF6 mRNA having Genbank Accession Number NM_{—}001160124.1 (SEQ ID NO: 91);
the KLF6 mRNA having Genbank Accession Number NM_{—}001160125.1 (SEQ ID NO: 93);
the KLF6 mRNA having Genbank Accession Number NM_{—}001300.5 (SEQ ID NO: 95);
the ACPP mRNA having Genbank Accession Number NM_{—}001134194.1 (SEQ ID NO: 99);
the ACPP mRNA having Genbank Accession Number NM_{—}001099.4 (SEQ ID NO: 97);
the IGFBP4 mRNA having Genbank Accession Number NM_{—}001552.2 (SEQ ID NO: 101);
the IGF1 mRNA having Genbank Accession Number NM_{—}000618.3 (SEQ ID NO: 103);
the IGF1 mRNA having Genbank Accession Number NM_{—}001111283.1 (SEQ ID NO: 105);
the IGF1 mRNA having Genbank Accession Number NM_{—}001111284.1 (SEQ ID NO: 107);
the IGF1 mRNA having Genbank Accession Number NM_{—}001111285.1 (SEQ ID NO: 109);
the IRS1 mRNA having Genbank Accession Number NM_{—}005544.2 (SEQ ID NO: 111);
the FOSL2 mRNA having Genbank Accession Number NM_{—}005253.3 (SEQ ID NO: 113);
the HRAS mRNA having Genbank Accession Number NM_{—}001130442.1 (SEQ ID NO: 115);
the HRAS mRNA having Genbank Accession Number NM_{—}005343.2 (SEQ ID NO: 117);
the HRAS mRNA having Genbank Accession Number NM_{—}176795.3 (SEQ ID NO: 119);
the CLU mRNA having Genbank Accession Number NM_{—}001171138.1 (SEQ ID NO: 121);
the CLU mRNA having Genbank Accession Number NM_{—}001831.2 (SEQ ID NO: 123);
the CLU mRNA having Genbank Accession Number NM_{—}203339.1 (SEQ ID NO: 125);
the HSD17B1 mRNA having Genbank Accession Number NM_{—}000413.2 (SEQ ID NO: 127);
the HSDL1 mRNA having Genbank Accession Number NM_{—}001146051.1 (SEQ ID NO: 129);
the HSDL1 mRNA having Genbank Accession Number NM_{—}031463.4 (SEQ ID NO: 131);
the HSD11B2 mRNA having Genbank Accession Number NM_{—}000196.3 (SEQ ID NO: 133);
the STC1 mRNA having Genbank Accession Number NM_{—}003155.2 (SEQ ID NO: 135);
the CYP11A1 mRNA having Genbank Accession Number NM_{—}000781.2 (SEQ ID NO: 137);
the CYP11A1 mRNA having Genbank Accession Number NM_{—}001099773.1 (SEQ ID NO: 139);
the GMNN mRNA having Genbank Accession Number NM_{—}015895.3 (SEQ ID NO: 141);
the HSD3B2 mRNA having Genbank Accession Number NM_{—}000198.3 (SEQ ID NO: 143);
the HSD3B2 mRNA having Genbank Accession Number NM_{—}001166120.1 (SEQ ID NO: 145);
the CYP19A1 mRNA having Genbank Accession Number NM_{—}000103.3 (SEQ ID NO: 147);
the CYP19A1 mRNA having Genbank Accession Number NM_{—}031226.2 (SEQ ID NO: 149);
the IGFBP5 mRNA having Genbank Accession Number NM_{—}000599.3 (SEQ ID NO: 151);
the KCNK3 mRNA having Genbank Accession Number NM_{—}002246.2 (SEQ ID NO: 153);
the SMAD7 mRNA having Genbank Accession Number NM_{—}001190821.1 (SEQ ID NO: 155);
the SMAD7 mRNA having Genbank Accession Number NM_{—}001190822.1 (SEQ ID NO: 157);
the SMAD7 mRNA having Genbank Accession Number NM_{—}001190823.1 (SEQ ID NO: 159);
the SMAD7 mRNA having Genbank Accession Number NM_{—}005904.3 (SEQ ID NO: 161);
the EGR3 mRNA having Genbank Accession Number NM_{—}001199880.1 (SEQ ID NO: 163);
the EGR3 mRNA having Genbank Accession Number NM_{—}001199881.1 (SEQ ID NO: 165);
the EGR3 mRNA having Genbank Accession Number NM_{—}004430.2 (SEQ ID NO: 167);
the FN1 mRNA having Genbank Accession Number NM_{—}002026.2 (SEQ ID NO: 169);
the FN1 mRNA having Genbank Accession Number NM_{—}054034.2 (SEQ ID NO: 171);
the FN1 mRNA having Genbank Accession Number NM_{—}212474.1 (SEQ ID NO: 173);
the FN1 mRNA having Genbank Accession Number NM_{—}212476.1 (SEQ ID NO: 175);
the FN1 mRNA having Genbank Accession Number NM_{—}212478.1 (SEQ ID NO: 177); and
the FN1 mRNA having Genbank Accession Number NM_{—}212482.1 (SEQ ID NO: 179).

19. The method according to claim 2 wherein the marker gene expression level is determined by determining the level of polypeptide produced from said marker gene.

20. The method according to claim 19 wherein the polypeptide level is determined by Western blot, enzyme-linked immunosorbent assay, radioimmunoassay, fluorescence assay, chemiluminescence assay and aptamer-based assay.

21. The method according to claim 19 wherein the mammalian oocyte is an oocyte of a domesticated mammal.

22. The method according to claim 21 wherein the domesticated mammal is a bovine, pig or sheep.

23. The method according to claim 22 wherein the domesticated mammal is a bovine.

24. The method according to claim 23 wherein the level of at least one of the following polypeptides is determined:
the NEK6 polypeptide having Genbank Accession Number NP_{—}001092458.1 (SEQ ID NO: 182);
the AQP11 polypeptide having Genbank Accession Number NP_{—}001103539.1 (SEQ ID NO: 184);
the CCDC126 polypeptide having Genbank Accession Number NP_{—}001075941.1 (SEQ ID NO: 186);
the KLF6 polypeptide having Genbank Accession Number NP_{—}001030348.2 (SEQ ID NO: 188);
the ACPP polypeptide having Genbank Accession Number NP_{—}001092336.1 (SEQ ID NO: 190);
the IGFBP4 polypeptide having Genbank Accession Number NP_{—}776982.1 (SEQ ID NO: 192);
the IGF1 polypeptide having Genbank Accession Number NP_{—}001071296.1 (SEQ ID NO: 194);
the IRS1 polypeptide having Genbank Accession Number XP_{—}581382.2 (SEQ ID NO: 196);
the IRS1 polypeptide having Genbank Accession Number XP_{—}002685688.1 (SEQ ID NO: 198);
the FOSL2 polypeptide having Genbank Accession Number NP_{—}001179879.1 (SEQ ID NO: 200);
the FOSL2 polypeptide having Genbank Accession Number XP_{—}002691497.1 (SEQ ID NO: 202);
the HRAS polypeptide having Genbank Accession Number XP_{—}590626.2 (SEQ ID NO: 204);
the HRAS polypeptide having Genbank Accession Number XP_{—}879748.1 (SEQ ID NO: 206);
the HRAS polypeptide having Genbank Accession Number XP_{—}879663.1 (SEQ ID NO: 208);
the CLU polypeptide having Genbank Accession Number NP_{—}776327.1 (SEQ ID NO: 210);
the HSD17B1 polypeptide having Genbank Accession Number XP_{—}001253408.1 (SEQ ID NO: 212);
the HSD17B1 polypeptide having Genbank Accession Number NP_{—}001095835.1 (SEQ ID NO: 214);
the HSDL1 polypeptide having Genbank Accession Number NP_{—}001092341.1 (SEQ ID NO: 216);
the HSD11B2 polypeptide having Genbank Accession Number NP_{—}777067.1 (SEQ ID NO: 218);
the STC1 polypeptide having Genbank Accession Number NP_{—}788842.2 (SEQ ID NO: 220);
the CYP11A1 polypeptide having Genbank Accession Number NP_{—}788817.1 (SEQ ID NO: 222);
the GMNN polypeptide having Genbank Accession Number NP_{—}001020508.1 (SEQ ID NO: 224);
the CYP19A1 polypeptide having Genbank Accession Number NP_{—}776730.1 (SEQ ID NO: 226);
the IGFBP5 polypeptide having Genbank Accession Number NP_{—}001098797.1 (SEQ ID NO: 228);
the KCNK3 polypeptide having Genbank Accession Number XP_{—}597401.4 (SEQ ID NO: 230);
the KCNK3 polypeptide having Genbank Accession Number XP_{—}002691504.1 (SEQ ID NO: 232);
the SMAD7 polypeptide having Genbank Accession Number NP_{—}001179794.1 (SEQ ID NO: 234);
the SMAD7 polypeptide having Genbank Accession Number XP_{—}002697809.1 (SEQ ID NO: 236);
the SMAD7 polypeptide having Genbank Accession Number XP_{—}616030.3 (SEQ ID NO: 238);
the EGR3 polypeptide having Genbank Accession Number XP_{—}604596.4 (SEQ ID NO: 240);
the EGR3 polypeptide having Genbank Accession Number XP_{—}002689819.1 (SEQ ID NO: 242); and
the FN1 polypeptide having Genbank Accession Number NP_{—}001157250.1 (SEQ ID NO: 244).

25. The method according to claim 22 wherein the domesticated mammal is a pig.

26. The method according to claim 25 wherein the level of at least one of the following polypeptides is determined:
the KLF6 polypeptide having Genbank Accession Number NP_{—}001127825.1 (SEQ ID NO: 246);
the IGFBP4 polypeptide having Genbank Accession Number NP_{—}001116601.1 (SEQ ID NO: 248);
the IGF1 polypeptide having Genbank Accession Number NP_{—}999421.1 (SEQ ID NO: 250);
the HRAS polypeptide having Genbank Accession Number NP_{—}001038002.1 (SEQ ID NO: 252);
the CLU polypeptide having Genbank Accession Number NP_{—}999136.1 (SEQ ID NO: 254);
the HSD17B1 polypeptide having Genbank Accession Number NP_{—}001121944.1 (SEQ ID NO: 256);
the HSD11B2 polypeptide having Genbank Accession Number NP_{—}999078.1 (SEQ ID NO: 258);
the STC1 polypeptide having Genbank Accession Number NP_{—}001096682.1 (SEQ ID NO: 260);
the CYP11A1 polypeptide having Genbank Accession Number NP_{—}999592.1 (SEQ ID NO: 262);
the CYP19A1 polypeptide having Genbank Accession Number NP_{—}999594.1 (SEQ ID NO: 264);
the IGFBP5 polypeptide having Genbank Accession Number NP_{—}999264.1 (SEQ ID NO: 266);
the SMAD7 polypeptide having Genbank Accession Number XP_{—}001927617.1 (SEQ ID NO: 268);
the ACPP polypeptide having Genbank Accession Number XP_{—}003132467.1 (SEQ ID NO: 270);
the IRS1 polypeptide having Genbank Accession Number ACG59405.1 (SEQ ID NO: 272);
the EGR3 polypeptide having Genbank Accession Number XP_{—}003132855.1 (SEQ ID NO: 274);
the FN1 polypeptide having Genbank Accession Number XP_{—}003133689.1 (SEQ ID NO: 276);
the FN1 polypeptide having Genbank Accession Number XP_{—}003133690.1 (SEQ ID NO: 278); and
the FN1 polypeptide having Genbank Accession Number XP_{—}003133691.1 (SEQ ID NO: 280).

27. The method according to claim 19 wherein the mammalian oocyte is an oocyte of a human being.

28. The method according to claim 24 wherein the level of at least one of the following polypeptides is determined:
the NEK6 polypeptide having Genbank Accession Number NP_{—}001138473.1 (SEQ ID NO: 74):
the NEK6 polypeptide having Genbank Accession Number NP_{—}001159639.1 (SEQ ID NO: 76);
the NEK6 polypeptide having Genbank Accession Number NP_{—}001159640.1 (SEQ ID NO: 78);
the NEK6 polypeptide having Genbank Accession Number NP_{—}001159641.1 (SEQ ID NO: 80);
the NEK6 polypeptide having Genbank Accession Number NP_{—}001159642.1 (SEQ ID NO: 82);
the NEK6 polypeptide having Genbank Accession Number NP_{—}001159643.1 (SEQ ID NO: 84);
the NEK6 polypeptide having Genbank Accession Number NP_{—}055212.2 (SEQ ID NO: 86);
the AQP11 polypeptide having Genbank Accession Number NP_{—}766627.1 (SEQ ID NO: 88);
the CCDC126 polypeptide having Genbank Accession Number NP_{—}620126.2 (SEQ ID NO: 90);
the KLF6 polypeptide having Genbank Accession Number NP_{—}001153596.1 (SEQ ID NO: 92);
the KLF6 polypeptide having Genbank Accession Number NP_{—}001153597.1 (SEQ ID NO: 94);
the KLF6 polypeptide having Genbank Accession Number NP_{—}001291.3 (SEQ ID NO: 96);
the ACPP polypeptide having Genbank Accession Number NP_{—}001127666.1 (SEQ ID NO: 100);
the ACPP polypeptide having Genbank Accession Number NP_{—}001090.2 (SEQ ID NO: 98);
the IGFBP4 polypeptide having Genbank Accession Number NP_{—}001543.2 (SEQ ID NO: 102);
the IGF1 polypeptide having Genbank Accession Number NP_{—}000609.1 (SEQ ID NO: 104);
the IGF1 polypeptide having Genbank Accession Number NP_{—}001104753.1 (SEQ ID NO: 106);
the IGF1 polypeptide having Genbank Accession Number NP_{—}001104754.1 (SEQ ID NO: 108);
the IGF1 polypeptide having Genbank Accession Number NP_{—}001104755.1 (SEQ ID NO: 110);
the IRS1 polypeptide having Genbank Accession Number NP_{—}005535.1 (SEQ ID NO: 112);
the FOSL2 polypeptide having Genbank Accession Number NP_{—}005244.1 (SEQ ID NO: 114);
the HRAS polypeptide having Genbank Accession Number NP_{—}001123914.1 (SEQ ID NO: 116);
the HRAS polypeptide having Genbank Accession Number NP_{—}005334.1 (SEQ ID NO: 118);
the HRAS polypeptide having Genbank Accession Number NP_{—}789765.1 (SEQ ID NO: 120);
the CLU polypeptide having Genbank Accession Number NP_{—}001164609.1 (SEQ ID NO: 122);
the CLU polypeptide having Genbank Accession Number NP_{—}001822.2 (SEQ ID NO: 124);
the CLU polypeptide having Genbank Accession Number NP_{—}976084.1 (SEQ ID NO: 126);
the HSD17B1 polypeptide having Genbank Accession Number NP_{—}000404.2 (SEQ ID NO: 128);
the HSDL1 polypeptide having Genbank Accession Number NP_{—}001139523.1 (SEQ ID NO: 130);
the HSDL1 polypeptide having Genbank Accession Number NP_{—}113651.4 (SEQ ID NO: 132);
the HSD11B2 polypeptide having Genbank Accession Number NP_{—}000187.3 (SEQ ID NO: 134);
the STC1 polypeptide having Genbank Accession Number NP_{—}003146.1 (SEQ ID NO: 136);
the CYP11A1 polypeptide having Genbank Accession Number NP_{—}000772.2 (SEQ ID NO: 138);
the CYP11A1 polypeptide having Genbank Accession Number NP_{—}001093243.1 (SEQ ID NO: 140);
the GMNN polypeptide having Genbank Accession Number NP_{—}056979.1 (SEQ ID NO: 142);
the HSD3B2 polypeptide having Genbank Accession Number NP_{—}000189.1 (SEQ ID NO: 144);
the HSD3B2 polypeptide having Genbank Accession Number NP_{—}001159592.1 (SEQ ID NO: 146);
the CYP19A1 polypeptide having Genbank Accession Number NP_{—}000094.2 (SEQ ID NO: 148);
the CYP19A1 polypeptide having Genbank Accession Number NP_{—}112503.1 (SEQ ID NO: 150);
the IGFBP5 polypeptide having Genbank Accession Number NP_{—}000590.1 (SEQ ID NO: 152);
the KCNK3 polypeptide having Genbank Accession Number NP_{—}002237.1 (SEQ ID NO: 154);
the SMAD7 polypeptide having Genbank Accession Number NP_{—}001177750.1 (SEQ ID NO: 156);
the SMAD7 polypeptide having Genbank Accession Number NP_{—}001177751.1 (SEQ ID NO: 158);
the SMAD7 polypeptide having Genbank Accession Number NP_{—}001177752.1 (SEQ ID NO: 160);
the SMAD7 polypeptide having Genbank Accession Number NP_{—}005895.1 (SEQ ID NO: 162);
the EGR3 polypeptide having Genbank Accession Number NP_{—}001186809.1 (SEQ ID NO: 164);
the EGR3 polypeptide having Genbank Accession Number NP_{—}001186810.1 (SEQ ID NO: 166);
the EGR3 polypeptide having Genbank Accession Number NP_{—}004421.2 (SEQ ID NO: 168);
the FN1 polypeptide having Genbank Accession Number NP_{—}002017.1 (SEQ ID NO: 170);
the FN1 polypeptide having Genbank Accession Number NP_{—}473375.2 (SEQ ID NO: 172);
the FN1 polypeptide having Genbank Accession Number NP_{—}997639.1 (SEQ ID NO: 174);
the FN1 polypeptide having Genbank Accession Number NP_{—}997641.1 (SEQ ID NO: 176);
the FN1 polypeptide having Genbank Accession Number NP_{—}997643.1 (SEQ ID NO: 178); and
the FN1 polypeptide having Genbank Accession Number NP_{—}997647.1 (SEQ ID NO: 180).

29. A method for selecting a mammalian oocyte from a plurality of candidate oocytes for preservation or implantation comprising:
determining the level of expression of at least one marker gene of a set of marker genes comprising ACPP, AQP11, CCDC126, CLU, CYP11A1, CYP19A1, EGR3, FN1, FOSL2, GMNN, HRAS, HSD3B2, HSD17B1, HSD11B2, HSDL1, IGF1, IGFBP4, IGFBP5, IRS 1, KCNK3, KLF6, NEK6, SMAD7 and STC 1 in each of a plurality of samples, each sample being derived from a cumulus cell or a granulosa cell associated with a candidate oocyte;
(a) comparing the expression level of said at least one marker gene in the plurality of samples;
(b) selecting for preservation or implantation a candidate oocyte associated with a sample having a level of marker gene expression compared to the level of marker gene expression in other samples, which level of marker gene expression of said selected candidate is indicative of a higher probability of oocyte quality than at least one other oocyte in the plurality of candidate oocytes.

30. The method according to claim 29 wherein the level of expression of at least three of said marker genes is determined.

31. The method according to claim 30 wherein the marker genes comprise NEK6, AQP11 and IGF1.

32. The method according to claim 31 wherein the probability of a mammalian oocyte of high quality (P) is given by the equation:
P=e^{5.608+0.645x+0.100y-2.17z}/1+e^{.}e^{5.608+0.645x+0.100y-2.17z}
wherein:
x is the expression level of NEK6 relative to a control;
y is the expression level of AQP11 relative to a control; and
z is the expression level of IGF1 relative to a control.

33. The method according to claim 30 wherein the level of expression of at least ten of said marker genes is determined.

34. The method according to claim 29 wherein the marker gene expression level is determined by determining the level of mRNA produced from said marker gene.

35. The method according to claim 34 wherein the level of mRNA is determined by reverse transcription polymerase chain reaction.

36. The method according to claim 34 wherein the mammalian oocyte is an oocyte of a domesticated mammal.

37. The method according to claim 36 wherein the domesticated mammal is a bovine, sheep or pig.

38. The method according to claim 37 wherein the domesticated mammal is a bovine.

39. The method according to claim 38 wherein the level of at least one of the following mRNAs is determined:
the NEK6 mRNA having Genbank Accession Number NM_{—}001098988.1 (SEQ ID NO: 181);
the AQP 11 mRNA having Genbank Accession Number NM_{—}001110069.1 (SEQ ID NO: 183);
the CCDC126 mRNA having Genbank Accession Number NM_{—}001082472.2 (SEQ ID NO: 185);
the KLF6 mRNA having Genbank Accession Number NM_{—}001035271.2 (SEQ ID NO: 187);
the ACPP mRNA having Genbank Accession Number NM_{—}001098866.1 (SEQ ID NO: 189);
the IGFBP4 mRNA having Genbank Accession Number NM_{—}174557.3 (SEQ ID NO: 191);
the IGF1 mRNA having Genbank Accession Number NM_{—}001077828.1 (SEQ ID NO: 193);
the IRS1 mRNA having Genbank Accession Number XM_{—}581382.3 (SEQ ID NO: 195);
the IRS1 mRNA having Genbank Accession Number XM_{—}002685642.1 (SEQ ID NO: 197);
the FOSL2 mRNA having Genbank Accession Number NM_{—}001192950.1 (SEQ ID NO: 199);
the FOSL2 mRNA having Genbank Accession Number XM_{—}002691451.1 (SEQ ID NO: 201);
the HRAS mRNA having Genbank Accession Number XM_{—}590626.2 (SEQ ID NO: 203);
the HRAS mRNA having Genbank Accession Number XM_{—}874655.1 (SEQ ID NO: 205);
the HRAS mRNA having Genbank Accession Number XM_{—}874570.1 (SEQ ID NO: 207);
the CLU mRNA having Genbank Accession Number NM_{—}173902.2 (SEQ ID NO: 209);
the HSD17B1 mRNA having Genbank Accession Number XM_{—}001253407.1 (SEQ ID NO: 211);
the HSD17B1 mRNA having Genbank Accession Number NM_{—}001102365.1 (SEQ ID NO: 213);
the HSDL1 mRNA having Genbank Accession Number NM_{—}001098871.1 (SEQ ID NO: 215);
the HSD11B2 mRNA having Genbank Accession Number NM_{—}174642.1 (SEQ ID NO: 217);
the STC1 mRNA having Genbank Accession Number NM_{—}176669.3 (SEQ ID NO: 219);
the CYP11A1 mRNA having Genbank Accession Number NM_{—}176644.2 (SEQ ID NO: 221);
the GMNN mRNA having Genbank Accession Number NM_{—}001025337.1 (SEQ ID NO: 223);
the CYP19A1 mRNA having Genbank Accession Number NM_{—}174305.1 (SEQ ID NO: 225);
the IGFBP5 mRNA having Genbank Accession Number NM_{—}001105327.1 (SEQ ID NO: 227);
the KCNK3 mRNA having Genbank Accession Number XM_{—}597401.5 (SEQ ID NO: 229);
the KCNK3 mRNA having Genbank Accession Number XM_{—}002691458.1 (SEQ ID NO: 231);
the SMAD7 mRNA having Genbank Accession Number NM_{—}001192865.1 (SEQ ID NO: 233);
the SMAD7 mRNA having Genbank Accession Number XM_{—}002697763.1 (SEQ ID NO: 235);
the SMAD7 mRNA having Genbank Accession Number XM_{—}616030.3 (SEQ ID NO: 237);
the EGR3 mRNA having Genbank Accession Number XM_{—}604596.5 (SEQ ID NO: 239);
the EGR3 mRNA having Genbank Accession Number XM_{—}002689773.1 (SEQ ID NO: 241); and
the FN1 mRNA having Genbank Accession Number NM_{—}001163778.1 (SEQ ID NO: 243).

40. The method according to claim 37 wherein the domesticated mammal is a pig.

41. The method according to claim 40 wherein the level of at least one of the following mRNAs is determined:
the KLF6 mRNA having Genbank Accession Number NM_{—}001134353.2 (SEQ ID NO: 245;
the IGFBP4 mRNA having Genbank Accession Number NM_{—}001123129.1 (SEQ ID NO: 247);
the IGF1 mRNA having Genbank Accession Number NM_{—}214256.1 (SEQ ID NO: 249);
the HRAS mRNA having Genbank Accession Number NM_{—}001044537.1 (SEQ ID NO: 251);
the CLU mRNA having Genbank Accession Number NM_{—}213971.1 (SEQ ID NO: 253);
the HSD17B1 mRNA having Genbank Accession Number NM_{—}001128472.1 (SEQ ID NO: 255);
the HSD11B2 mRNA having Genbank Accession Number NM_{—}213913.1 (SEQ ID NO: 257);
the STC1 mRNA having Genbank Accession Number NM_{—}001103212.1 (SEQ ID NO: 259);
the CYP11A1 mRNA having Genbank Accession Number NM_{—}214427.1 (SEQ ID NO: 261);
the CYP19A1 mRNA having Genbank Accession Number NM_{—}214429.1 (SEQ ID NO: 263);
the IGFBP5 mRNA having Genbank Accession Number NM_{—}214099.1 (SEQ ID NO: 265);
the SMAD7 mRNA having Genbank Accession Number XM_{—}001927582.1 (SEQ ID NO: 267);
the ACPP mRNA having Genbank Accession Number XM_{—}003132419.1 (SEQ ID NO: 269);
the IRS1 mRNA having Genbank Accession Number EU681268.1 (SEQ ID NO: 271);
the EGR3 mRNA having Genbank Accession Number XM_{—}003132807.1 (SEQ ID NO: 273);
the FN1 mRNA having Genbank Accession Number XM_{—}003133641.1 (SEQ ID NO: 275);
the FN1 mRNA having Genbank Accession Number XM_{—}003133642.1 (SEQ ID NO: 277); and
the FN1 mRNA having Genbank Accession Number XM_{—}003133643.1 (SEQ ID NO: 279).

42. The method according to claim 34 wherein the mammalian oocyte is an oocyte of a human being.

43. The method according to claim 42 wherein the level of at least one of the following mRNAs is determined:
the NEK6 mRNA having Genbank Accession Number NM_{—}001145001.2 (SEQ ID NO: 73;
the NEK6 mRNA having Genbank Accession Number NM_{—}001166167.1 (SEQ ID NO: 75);
the NEK6 mRNA having Genbank Accession Number NM_{—}001166168.1 (SEQ ID NO: 77);
the NEK6 mRNA having Genbank Accession Number NM_{—}001166169.1 (SEQ ID NO: 79);
the NEK6 mRNA having Genbank Accession Number NM_{—}001166170.1 (SEQ ID NO: 81);
the NEK6 mRNA having Genbank Accession Number NM_{—}001166171.1 (SEQ ID NO: 83);
the NEK6 mRNA having Genbank Accession Number NM_{—}014397.5 (SEQ ID NO: 85);
the AQP11 mRNA having Genbank Accession Number NM_{—}173039.2 (SEQ ID NO: 87);
the CCDC126 mRNA having Genbank Accession Number NM_{—}138771.3 (SEQ ID NO: 89);
the KLF6 mRNA having Genbank Accession Number NM_{—}001160124.1 (SEQ ID NO: 91);
the KLF6 mRNA having Genbank Accession Number NM_{—}001160125.1 (SEQ ID NO: 93);
the KLF6 mRNA having Genbank Accession Number NM_{—}001300.5 (SEQ ID NO: 95);
the ACPP mRNA having Genbank Accession Number NM_{—}001134194.1 (SEQ ID NO: 99);
the ACPP mRNA having Genbank Accession Number NM_{—}001099.4 (SEQ ID NO: 97);
the IGFBP4 mRNA having Genbank Accession Number NM_{—}001552.2 (SEQ ID NO: 101);
the IGF1 mRNA having Genbank Accession Number NM_{—}000618.3 (SEQ ID NO: 103);
the IGF1 mRNA having Genbank Accession Number NM_{—}001111283.1 (SEQ ID NO: 105);
the IGF1 mRNA having Genbank Accession Number NM_{—}001111284.1 (SEQ ID NO: 107);
the IGF1 mRNA having Genbank Accession Number NM_{—}001111285.1 (SEQ ID NO: 109);
the IRS1 mRNA having Genbank Accession Number NM_{—}005544.2 (SEQ ID NO: 111);
the FOSL2 mRNA having Genbank Accession Number NM_{—}005253.3 (SEQ ID NO: 113);
the HRAS mRNA having Genbank Accession Number NM_{—}001130442.1 (SEQ ID NO: 115);
the HRAS mRNA having Genbank Accession Number NM_{—}005343.2 (SEQ ID NO: 117);
the HRAS mRNA having Genbank Accession Number NM_{—}176795.3 (SEQ ID NO: 119);
the CLU mRNA having Genbank Accession Number NM_{—}001171138.1 (SEQ ID NO: 121);
the CLU mRNA having Genbank Accession Number NM_{—}001831.2 (SEQ ID NO: 123);
the CLU mRNA having Genbank Accession Number NM_{—}203339.1 (SEQ ID NO: 125);
the HSD17B1 mRNA having Genbank Accession Number NM_{—}000413.2 (SEQ ID NO: 127);
the HSDL1 mRNA having Genbank Accession Number NM_{—}001146051.1 (SEQ ID NO: 129);
the HSDL1 mRNA having Genbank Accession Number NM_{—}031463.4 (SEQ ID NO: 131);
the HSD11B2 mRNA having Genbank Accession Number NM_{—}000196.3 (SEQ ID NO: 133);
the STC1 mRNA having Genbank Accession Number NM_{—}003155.2 (SEQ ID NO: 135);
the CYP11A1 mRNA having Genbank Accession Number NM_{—}000781.2 (SEQ ID NO: 137);
the CYP11A1 mRNA having Genbank Accession Number NM_{—}001099773.1 (SEQ ID NO: 139);
the GMNN mRNA having Genbank Accession Number NM_{—}015895.3 (SEQ ID NO: 141);
the HSD3B2 mRNA having Genbank Accession Number NM_{—}000198.3 (SEQ ID NO: 143);
the HSD3B2 mRNA having Genbank Accession Number NM_{—}001166120.1 (SEQ ID NO: 145);
the CYP19A1 mRNA having Genbank Accession Number NM_{—}000103.3 (SEQ ID NO: 147);
the CYP19A1 mRNA having Genbank Accession Number NM_{—}031226.2 (SEQ ID NO: 149);
the IGFBP5 mRNA having Genbank Accession Number NM_{—}000599.3 (SEQ ID NO: 151);
the KCNK3 mRNA having Genbank Accession Number NM_{—}002246.2 (SEQ ID NO: 153);
the SMAD7 mRNA having Genbank Accession Number NM_{—}001190821.1 (SEQ ID NO: 155);
the SMAD7 mRNA having Genbank Accession Number NM_{—}001190822.1 (SEQ ID NO: 157);
the SMAD7 mRNA having Genbank Accession Number NM_{—}001190823.1 (SEQ ID NO: 159);
the SMAD7 mRNA having Genbank Accession Number NM_{—}005904.3 (SEQ ID NO: 161);
the EGR3 mRNA having Genbank Accession Number NM_{—}001199880.1 (SEQ ID NO: 163);
the EGR3 mRNA having Genbank Accession Number NM_{—}001199881.1 (SEQ ID NO: 165);
the EGR3 mRNA having Genbank Accession Number NM_{—}004430.2 (SEQ ID NO: 167);
the FN1 mRNA having Genbank Accession Number NM_{—}002026.2 (SEQ ID NO: 169);
the FN1 mRNA having Genbank Accession Number NM_{—}054034.2 (SEQ ID NO: 171);
the FN1 mRNA having Genbank Accession Number NM_{—}212474.1 (SEQ ID NO: 173);
the FN1 mRNA having Genbank Accession Number NM_{—}212476.1 (SEQ ID NO: 175);
the FN1 mRNA having Genbank Accession Number NM_{—}212478.1 (SEQ ID NO: 177); and
the FN1 mRNA having Genbank Accession Number NM_{—}212482.1 (SEQ ID NO: 179).

44. The method according to claim 29 wherein the marker gene expression level is determined by determining the level of polypeptide produced from said marker gene.

45. The method according to claim 44 wherein the mammalian oocyte is an oocyte of a domesticated mammal.

46. The method according to claim 45 wherein the domesticated mammal is a bovine.

47. The method according to claim 46 wherein the level of at least one of the following polypeptides is determined:
the NEK6 polypeptide having Genbank Accession Number NP_{—}001092458.1 (SEQ ID NO: 182);
the AQP11 polypeptide having Genbank Accession Number NP_{—}001103539.1 (SEQ ID NO: 184);
the CCDC126 polypeptide having Genbank Accession Number NP_{—}001075941.1 (SEQ ID NO: 186);
the KLF6 polypeptide having Genbank Accession Number NP_{—}001030348.2 (SEQ ID NO: 188);
the ACPP polypeptide having Genbank Accession Number NP_{—}001092336.1 (SEQ ID NO: 190);
the IGFBP4 polypeptide having Genbank Accession Number NP_{—}776982.1 (SEQ ID NO: 192);
the IGF1 polypeptide having Genbank Accession Number NP_{—}001071296.1 (SEQ ID NO: 194);
the IRS 1 polypeptide having Genbank Accession Number XP_{—}581382.2 (SEQ ID NO: 196);
the IRS1 polypeptide having Genbank Accession Number XP_{—}002685688.1 (SEQ ID NO: 198);
the FOSL2 polypeptide having Genbank Accession Number NP_{—}001179879.1 (SEQ ID NO: 200);
the FOSL2 polypeptide having Genbank Accession Number XP_{—}002691497.1 (SEQ ID NO: 202);
the HRAS polypeptide having Genbank Accession Number XP_{—}590626.2 (SEQ ID NO: 204);
the HRAS polypeptide having Genbank Accession Number XP_{—}879748.1 (SEQ ID NO: 206);
the HRAS polypeptide having Genbank Accession Number XP_{—}879663.1 (SEQ ID NO: 208);
the CLU polypeptide having Genbank Accession Number NP_{—}776327.1 (SEQ ID NO: 210);
the HSD17B1 polypeptide having Genbank Accession Number XP_{—}001253408.1 (SEQ ID NO: 212);
the HSD17B1 polypeptide having Genbank Accession Number NP_{—}001095835.1 (SEQ ID NO: 214);
the HSDL1 polypeptide having Genbank Accession Number NP_{—}001092341.1 (SEQ ID NO: 216);
the HSD11B2 polypeptide having Genbank Accession Number NP_{—}777067.1 (SEQ ID NO: 218);
the STC1 polypeptide having Genbank Accession Number NP_{—}788842.2 (SEQ ID NO: 220);
the CYP11A1 polypeptide having Genbank Accession Number NP_{—}788817.1 (SEQ ID NO: 222);
the GMNN polypeptide having Genbank Accession Number NP_{—}001020508.1 (SEQ ID NO: 224);
the CYP19A1 polypeptide having Genbank Accession Number NP_{—}776730.1 (SEQ ID NO: 226);
the IGFBP5 polypeptide having Genbank Accession Number NP_{—}001098797.1 (SEQ ID NO: 228);
the KCNK3 polypeptide having Genbank Accession Number XP_{—}597401.4 (SEQ ID NO: 230);
the KCNK3 polypeptide having Genbank Accession Number XP_{—}002691504.1 (SEQ ID NO: 232);
the SMAD7 polypeptide having Genbank Accession Number NP_{—}001179794.1 (SEQ ID NO: 234);
the SMAD7 polypeptide having Genbank Accession Number XP_{—}002697809.1 (SEQ ID NO: 236);
the SMAD7 polypeptide having Genbank Accession Number XP_{—}616030.3 (SEQ ID NO: 238);
the EGR3 polypeptide having Genbank Accession Number XP_{—}604596.4 (SEQ ID NO: 240);
the EGR3 polypeptide having Genbank Accession Number XP_{—}002689819.1 (SEQ ID NO: 242); and
the FN1 polypeptide having Genbank Accession Number NP_{—}001157250.1 (SEQ ID NO: 244).

48. The method according to claim 45 wherein the domesticated mammal is a pig.

49. The method according to claim 48 wherein the level of at least one of the following polypeptides is determined:
the KLF6 polypeptide having Genbank Accession Number NP_{—}001127825.1 (SEQ ID NO: 246);
the IGFBP4 polypeptide having Genbank Accession Number NP_{—}001116601.1 (SEQ ID NO: 248);
the IGF1 polypeptide having Genbank Accession Number NP_{—}999421.1 (SEQ ID NO: 250);
the HRAS polypeptide having Genbank Accession Number NP_{—}001038002.1 (SEQ ID NO: 252);
the CLU polypeptide having Genbank Accession Number NP_{—}999136.1 (SEQ ID NO: 254);
the HSD17B1 polypeptide having Genbank Accession Number NP_{—}001121944.1 (SEQ ID NO: 256);
the HSD11B2 polypeptide having Genbank Accession Number NP_{—}999078.1 (SEQ ID NO: 258);
the STC1 polypeptide having Genbank Accession Number NP_{—}001096682.1 (SEQ ID NO: 260);
the CYP11A1 polypeptide having Genbank Accession Number NP_{—}999592.1 (SEQ ID NO: 262);
the CYP19A1 polypeptide having Genbank Accession Number NP_{—}999594.1 (SEQ ID NO: 264);
the IGFBP5 polypeptide having Genbank Accession Number NP_{—}999264.1 (SEQ ID NO: 266);
the SMAD7 polypeptide having Genbank Accession Number XP_{—}001927617.1 (SEQ ID NO: 268);
the ACPP polypeptide having Genbank Accession Number XP_{—}003132467.1 (SEQ ID NO: 270);
the IRS1 polypeptide having Genbank Accession Number ACG59405.1 (SEQ ID NO: 272);
the EGR3 polypeptide having Genbank Accession Number XP_{—}003132855.1 (SEQ ID NO: 274);
the FN1 polypeptide having Genbank Accession Number XP_{—}003133689.1 (SEQ ID NO: 276);
the FN1 polypeptide having Genbank Accession Number XP_{—}003133690.1 (SEQ ID NO: 278); and
the FN1 polypeptide having Genbank Accession Number XP_{—}003133691.1 (SEQ ID NO: 280).

50. The method according to claim 44 wherein the mammalian oocyte is an oocyte of a human being.

51. The method according to claim 50 wherein the level of at least one of the following polypeptides is determined:
the NEK6 polypeptide having Genbank Accession Number NP_{—}001138473.1 (SEQ ID NO: 74):
the NEK6 polypeptide having Genbank Accession Number NP_{—}001159639.1 (SEQ ID NO: 76);
the NEK6 polypeptide having Genbank Accession Number NP_{—}001159640.1 (SEQ ID NO: 78);
the NEK6 polypeptide having Genbank Accession Number NP_{—}001159641.1 (SEQ ID NO: 80);
the NEK6 polypeptide having Genbank Accession Number NP_{—}001159642.1 (SEQ ID NO: 82);
the NEK6 polypeptide having Genbank Accession Number NP_{—}001159643.1 (SEQ ID NO: 84);
the NEK6 polypeptide having Genbank Accession Number NP_{—}055212.2 (SEQ ID NO: 86);
the AQP11 polypeptide having Genbank Accession Number NP_{—}766627.1 (SEQ ID NO: 88);
the CCDC126 polypeptide having Genbank Accession Number NP_{—}620126.2 (SEQ ID NO: 90);
the KLF6 polypeptide having Genbank Accession Number NP_{—}001153596.1 (SEQ ID NO: 92);
the KLF6 polypeptide having Genbank Accession Number NP_{—}001153597.1 (SEQ ID NO: 94);
the KLF6 polypeptide having Genbank Accession Number NP_{—}001291.3 (SEQ ID NO: 96);
the ACPP polypeptide having Genbank Accession Number NP_{—}001127666.1 (SEQ ID NO: 100);
the ACPP polypeptide having Genbank Accession Number NP_{—}001090.2 (SEQ ID NO: 98);
the IGFBP4 polypeptide having Genbank Accession Number NP_{—}001543.2 (SEQ ID NO: 102);
the IGF1 polypeptide having Genbank Accession Number NP_{—}000609.1 (SEQ ID NO: 104);
the IGF1 polypeptide having Genbank Accession Number NP_{—}001104753.1 (SEQ ID NO: 106);
the IGF1 polypeptide having Genbank Accession Number NP_{—}001104754.1 (SEQ ID NO: 108);
the IGF1 polypeptide having Genbank Accession Number NP_{—}001104755.1 (SEQ ID NO: 110);
the IRS1 polypeptide having Genbank Accession Number NP_{—}005535.1 (SEQ ID NO: 112);
the FOSL2 polypeptide having Genbank Accession Number NP_{—}005244.1 (SEQ ID NO: 114);
the HRAS polypeptide having Genbank Accession Number NP_{—}001123914.1 (SEQ ID NO: 116);
the HRAS polypeptide having Genbank Accession Number NP_{—}005334.1 (SEQ ID NO: 118);
the HRAS polypeptide having Genbank Accession Number NP_{—}789765.1 (SEQ ID NO: 120);
the CLU polypeptide having Genbank Accession Number NP_{—}001164609.1 (SEQ ID NO: 122);
the CLU polypeptide having Genbank Accession Number NP_{—}001822.2 (SEQ ID NO: 124);
the CLU polypeptide having Genbank Accession Number NP_{—}976084.1 (SEQ ID NO: 126);
the HSD17B1 polypeptide having Genbank Accession Number NP_{—}000404.2 (SEQ ID NO: 128);
the HSDL1 polypeptide having Genbank Accession Number NP_{—}001139523.1 (SEQ ID NO: 130);
the HSDL1 polypeptide having Genbank Accession Number NP_{—}113651.4 (SEQ ID NO: 132);
the HSD11B2 polypeptide having Genbank Accession Number NP_{—}000187.3 (SEQ ID NO: 134);
the STC1 polypeptide having Genbank Accession Number NP_{—}003146.1 (SEQ ID NO: 136);
the CYP11A1 polypeptide having Genbank Accession Number NP_{—}000772.2 (SEQ ID NO: 138);
the CYP11A1 polypeptide having Genbank Accession Number NP_{—}001093243.1 (SEQ ID NO: 140);
the GMNN polypeptide having Genbank Accession Number NP_{—}056979.1 (SEQ ID NO: 142);
the HSD3B2 polypeptide having Genbank Accession Number NP_{—}000189.1 (SEQ ID NO: 144);
the HSD3B2 polypeptide having Genbank Accession Number NP_{—}001159592.1 (SEQ ID NO: 146);
the CYP19A1 polypeptide having Genbank Accession Number NP_{—}000094.2 (SEQ ID NO: 148);
the CYP19A1 polypeptide having Genbank Accession Number NP_{—}112503.1 (SEQ ID NO: 150);
the IGFBP5 polypeptide having Genbank Accession Number NP_{—}000590.1 (SEQ ID NO: 152);
the KCNK3 polypeptide having Genbank Accession Number NP_{—}002237.1 (SEQ ID NO: 154);
the SMAD7 polypeptide having Genbank Accession Number NP_{—}001177750.1 (SEQ ID NO: 156);
the SMAD7 polypeptide having Genbank Accession Number NP_{—}001177751.1 (SEQ ID NO: 158);
the SMAD7 polypeptide having Genbank Accession Number NP_{—}001177752.1 (SEQ ID NO: 160);
the SMAD7 polypeptide having Genbank Accession Number NP_{—}005895.1 (SEQ ID NO: 162);
the EGR3 polypeptide having Genbank Accession Number NP_{—}001186809.1 (SEQ ID NO: 164);
the EGR3 polypeptide having Genbank Accession Number NP_{—}001186810.1 (SEQ ID NO: 166);
the EGR3 polypeptide having Genbank Accession Number NP_{—}004421.2 (SEQ ID NO: 168);
the FN1 polypeptide having Genbank Accession Number NP_{—}002017.1 (SEQ ID NO: 170);
the FN1 polypeptide having Genbank Accession Number NP_{—}473375.2 (SEQ ID NO: 172);
the FN1 polypeptide having Genbank Accession Number NP_{—}997639.1 (SEQ ID NO: 174);
the FN1 polypeptide having Genbank Accession Number NP_{—}997641.1 (SEQ ID NO: 176);
the FN1 polypeptide having Genbank Accession Number NP_{—}997643.1 (SEQ ID NO: 178); and
the FN1 polypeptide having Genbank Accession Number NP_{—}997647.1 (SEQ ID NO: 180).

52. A kit for evaluating mammalian oocyte quality comprising: a set of reagents that specifically detects the expression levels of one or more marker genes of a mammal comprising ACPP, AQP11, CCDC126, CLU, CYP11A1, CYP19A1, EGR3, FN1, FOSL2, GMNN, HRAS, HSD3B2, HSD17B1, HSD11B2, HSDL1, IGF1, IGFBP4, IGFBP5, IRS1, KCNK3, KLF6, NEK6, SMAD7 or STC1; and instructions for using said kit for evaluating oocyte quality.

53. The kit of claim 52, wherein the set of reagents detects mRNA expressed from said one or more marker genes.

54. The kit of claim 53, wherein the set of reagents comprises nucleic acid probes complementary to mRNA expressed from said one or more marker genes.

55. The kit of claim 54, wherein the nucleic acid probes complementary to mRNA are immobilized on a substrate surface.

56. The kit according to claim 53 wherein the set of reagents detect mRNA expressed from one or more marker genes of a domesticated mammal.

57. The kit according to claim 56 wherein the domesticated mammal is a bovine, sheep or pig.

58. The kit according to claim 57 wherein the domesticated mammal is a bovine.

59. The kit according to claim 58 wherein the set of reagents detects one or more of the following mRNAs:
the NEK6 mRNA having Genbank Accession Number NM_{—}001098988.1 (SEQ ID NO: 181);
the AQP11 mRNA having Genbank Accession Number NM_{—}001110069.1 (SEQ ID NO: 183);
the CCDC126 mRNA having Genbank Accession Number NM_{—}001082472.2 (SEQ ID NO: 185);
the KLF6 mRNA having Genbank Accession Number NM_{—}001035271.2 (SEQ ID NO: 187);
the ACPP mRNA having Genbank Accession Number NM_{—}001098866.1 (SEQ ID NO: 189);
the IGFBP4 mRNA having Genbank Accession Number NM_{—}174557.3 (SEQ ID NO: 191);
the IGF1 mRNA having Genbank Accession Number NM_{—}001077828.1 (SEQ ID NO: 193);
the IRS1 mRNA having Genbank Accession Number XM_{—}581382.3 (SEQ ID NO: 195);
the IRS1 mRNA having Genbank Accession Number XM_{—}002685642.1 (SEQ ID NO: 197);
the FOSL2 mRNA having Genbank Accession Number NM_{—}001192950.1(SEQ ID NO: 199);
the FOSL2 mRNA having Genbank Accession Number XM_{—}002691451.1(SEQ ID NO: 201);
the HRAS mRNA having Genbank Accession Number XM_{—}590626.2 (SEQ ID NO: 203);
the HRAS mRNA having Genbank Accession Number XM_{—}874655.1 (SEQ ID NO: 205);
the HRAS mRNA having Genbank Accession Number XM_{—}874570.1 (SEQ ID NO: 207);
the CLU mRNA having Genbank Accession Number NM_{—}173902.2 (SEQ ID NO: 209);
the HSD17B1 mRNA having Genbank Accession Number XM_{—}001253407.1 (SEQ ID NO: 211);
the HSD17B1 mRNA having Genbank Accession Number NM_{—}001102365.1 (SEQ ID NO: 213);
the HSDL1 mRNA having Genbank Accession Number NM_{—}001098871.1 (SEQ ID NO: 215);
the HSD11B2 mRNA having Genbank Accession Number NM_{—}174642.1 (SEQ ID NO: 217);
the STC1 mRNA having Genbank Accession Number NM_{—}176669.3 (SEQ ID NO: 219);
the CYP11A1 mRNA having Genbank Accession Number NM_{—}176644.2 (SEQ ID NO: 221);
the GMNN mRNA having Genbank Accession Number NM_{—}001025337.1 (SEQ ID NO: 223);
the CYP19A1 mRNA having Genbank Accession Number NM_{—}174305.1 (SEQ ID NO: 225);
the IGFBP5 mRNA having Genbank Accession Number NM_{—}001105327.1 (SEQ ID NO: 227);
the KCNK3 mRNA having Genbank Accession Number XM_{—}597401.5 (SEQ ID NO: 229);
the KCNK3 mRNA having Genbank Accession Number XM_{—}002691458.1 (SEQ ID NO: 231);
the SMAD7 mRNA having Genbank Accession Number NM_{—}001192865.1 (SEQ ID NO: 233);
the SMAD7 mRNA having Genbank Accession Number XM_{—}002697763.1 (SEQ ID NO: 235);
the SMAD7 mRNA having Genbank Accession Number XM_{—}616030.3 (SEQ ID NO: 237);
the EGR3 mRNA having Genbank Accession Number XM_{—}604596.5 (SEQ ID NO: 239);
the EGR3 mRNA having Genbank Accession Number XM_{—}002689773.1 (SEQ ID NO: 241); and
the FN1 mRNA having Genbank Accession Number NM_{—}001163778.1 (SEQ ID NO: 243).

60. The kit according to claim 57 wherein the domesticated mammal is a pig.

61. The method according to claim 60 wherein the level of at least one of the following mRNAs is determined:
the KLF6 mRNA having Genbank Accession Number NM_{—}001134353.2 (SEQ ID NO: 245);
the IGFBP4 mRNA having Genbank Accession Number NM_{—}001123129.1 (SEQ ID NO: 247);
the IGF1 mRNA having Genbank Accession Number NM_{—}214256.1 (SEQ ID NO: 249);
the HRAS mRNA having Genbank Accession Number NM_{—}001044537.1 (SEQ ID NO: 251);
the CLU mRNA having Genbank Accession Number NM_{—}213971.1 (SEQ ID NO: 253);
the HSD17B1 mRNA having Genbank Accession Number NM_{—}001128472.1 (SEQ ID NO: 255);
the HSD11B2 mRNA having Genbank Accession Number NM_{—}213913.1 (SEQ ID NO: 257);
the STC1 mRNA having Genbank Accession Number NM_{—}001103212.1 (SEQ ID NO: 259);
the CYP11A1 mRNA having Genbank Accession Number NM_{—}214427.1 (SEQ ID NO: 261);
the CYP19A1 mRNA having Genbank Accession Number NM_{—}214429.1 (SEQ ID NO: 263);
the IGFBP5 mRNA having Genbank Accession Number NM_{—}214099.1 (SEQ ID NO: 265);
the SMAD7 mRNA having Genbank Accession Number XM_{—}001927582.1 (SEQ ID NO: 267);
the ACPP mRNA having Genbank Accession Number XM_{—}003132419.1 (SEQ ID NO: 269);
the IRS1 mRNA having Genbank Accession Number EU681268.1 (SEQ ID NO: 271);
the EGR3 mRNA having Genbank Accession Number XM_{—}003132807.1 (SEQ ID NO: 273);
the FN1 mRNA having Genbank Accession Number XM_{—}003133641.1 (SEQ ID NO: 275);
the FN1 mRNA having Genbank Accession Number XM_{—}003133642.1 (SEQ ID NO: 277); and
the FN1 mRNA having Genbank Accession Number XM_{—}003133643.1 (SEQ ID NO: 279).

62. The kit according to claim 53 wherein the set of reagents detects mRNA expressed from one or more marker genes of a human being.

63. The kit according to claim 62 wherein the set of reagents detects one or more of the following mRNAs:
the NEK6 mRNA having Genbank Accession Number NM_{—}001145001.2 (SEQ ID NO: 73);
the NEK6 mRNA having Genbank Accession Number NM_{—}001166167.1 (SEQ ID NO: 75);
the NEK6 mRNA having Genbank Accession Number NM_{—}001166168.1 (SEQ ID NO: 77);
the NEK6 mRNA having Genbank Accession Number NM_{—}001166169.1 (SEQ ID NO: 79);
the NEK6 mRNA having Genbank Accession Number NM_{—}001166170.1 (SEQ ID NO: 81);
the NEK6 mRNA having Genbank Accession Number NM_{—}001166171.1 (SEQ ID NO: 83);
the NEK6 mRNA having Genbank Accession Number NM_{—}014397.5 (SEQ ID NO: 85);
the AQP11 mRNA having Genbank Accession Number NM_{—}173039.2 (SEQ ID NO: 87);
the CCDC126 mRNA having Genbank Accession Number NM_{—}138771.3 (SEQ ID NO: 89);
the KLF6 mRNA having Genbank Accession Number NM_{—}001160124.1 (SEQ ID NO: 91);
the KLF6 mRNA having Genbank Accession Number NM_{—}01160125.1 (SEQ ID NO: 93);
the KLF6 mRNA having Genbank Accession Number NM_{—}001300.5 (SEQ ID NO: 95);
the ACPP mRNA having Genbank Accession Number NM_{—}001134194.1 (SEQ ID NO: 99);
the ACPP mRNA having Genbank Accession Number NM_{—}001099.4 (SEQ ID NO: 97);
the IGFBP4 mRNA having Genbank Accession Number NM_{—}001552.2 (SEQ ID NO: 101);
the IGF1 mRNA having Genbank Accession Number NM_{—}000618.3 (SEQ ID NO: 103);
the IGF1 mRNA having Genbank Accession Number NM_{—}001111283.1 (SEQ ID NO: 105);
the IGF1 mRNA having Genbank Accession Number NM_{—}001111284.1 (SEQ ID NO: 107);
the IGF1 mRNA having Genbank Accession Number NM_{—}001111285.1 (SEQ ID NO: 109);
the IRS1 mRNA having Genbank Accession Number NM_{—}005544.2 (SEQ ID NO: 111);
the FOSL2 mRNA having Genbank Accession Number NM_{—}005253.3 (SEQ ID NO: 113);
the HRAS mRNA having Genbank Accession Number NM_{—}001130442.1 (SEQ ID NO: 115);
the HRAS mRNA having Genbank Accession Number NM_{—}005343.2 (SEQ ID NO: 117);
the HRAS mRNA having Genbank Accession Number NM_{—}176795.3 (SEQ ID NO: 119);
the CLU mRNA having Genbank Accession Number NM_{—}001171138.1 (SEQ ID NO: 121);
the CLU mRNA having Genbank Accession Number NM_{—}001831.2 (SEQ ID NO: 123);
the CLU mRNA having Genbank Accession Number NM_{—}203339.1 (SEQ ID NO: 125);
the HSD17B1 mRNA having Genbank Accession Number NM_{—}000413.2 (SEQ ID NO: 127);
the HSDL1 mRNA having Genbank Accession Number NM_{—}001146051.1 (SEQ ID NO: 129);
the HSDL1 mRNA having Genbank Accession Number NM_{—}031463.4 (SEQ ID NO: 131);
the HSD11B2 mRNA having Genbank Accession Number NM_{—}000196.3 (SEQ ID NO: 133);
the STC1 mRNA having Genbank Accession Number NM_{—}003155.2 (SEQ ID NO: 135);
the CYP11A1 mRNA having Genbank Accession Number NM_{—}000781.2 (SEQ ID NO: 137);
the CYP11A1 mRNA having Genbank Accession Number NM_{—}001099773.1 (SEQ ID NO: 139);
the GMNN mRNA having Genbank Accession Number NM_{—}015895.3 (SEQ ID NO: 141);
the HSD3B2 mRNA having Genbank Accession Number NM_{—}000198.3 (SEQ ID NO: 143);
the HSD3B2 mRNA having Genbank Accession Number NM_{—}001166120.1 (SEQ ID NO: 145);
the CYP19A1 mRNA having Genbank Accession Number NM_{—}000103.3 (SEQ ID NO: 147);
the CYP19A1 mRNA having Genbank Accession Number NM_{—}031226.2 (SEQ ID NO: 149);
the IGFBP5 mRNA having Genbank Accession Number NM_{—}000599.3 (SEQ ID NO: 151);
the KCNK3 mRNA having Genbank Accession Number NM_{—}002246.2 (SEQ ID NO: 153);
the SMAD7 mRNA having Genbank Accession Number NM_{—}001190821.1 (SEQ ID NO: 155);
the SMAD7 mRNA having Genbank Accession Number NM_{—}001190822.1 (SEQ ID NO: 157);
the SMAD7 mRNA having Genbank Accession Number NM_{—}001190823.1 (SEQ ID NO: 159);
the SMAD7 mRNA having Genbank Accession Number NM_{—}005904.3 (SEQ ID NO: 161);
the EGR3 mRNA having Genbank Accession Number NM_{—}001199880.1 (SEQ ID NO: 163);
the EGR3 mRNA having Genbank Accession Number NM_{—}001199881.1 (SEQ ID NO: 165);
the EGR3 mRNA having Genbank Accession Number NM_{—}004430.2 (SEQ ID NO: 167);
the FN1 mRNA having Genbank Accession Number NM_{—}002026.2 (SEQ ID NO: 169);
the FN1 mRNA having Genbank Accession Number NM_{—}054034.2 (SEQ ID NO: 171);
the FN1 mRNA having Genbank Accession Number NM_{—}212474.1 (SEQ ID NO: 173);
the FN1 mRNA having Genbank Accession Number NM_{—}212476.1 (SEQ ID NO: 175);
the FN1 mRNA having Genbank Accession Number NM_{—}212478.1 (SEQ ID NO: 177); and
the FN1 mRNA having Genbank Accession Number NM_{—}212482.1 (SEQ ID NO: 179).

64. The kit of claim 52, wherein the set of reagents detects polypeptides encoded by said one or more marker genes.

65. The kit of claim 64, wherein the set of reagents comprises antibodies or aptamers that specifically bind to the polypeptides encoded by said one or more marker genes.

66. The kit according to claim 64 wherein the set of reagents detects polypeptides encoded by one or more marker genes of a domesticated mammal.

67. The kit according to claim 66 wherein the domesticated mammal is a bovine, sheep or pig.

68. The kit according to claim 67 wherein the domesticated mammal is a bovine,

69. The kit according to claim 68 wherein the set of reagents detects one or more of the following polypeptides:
the NEK6 polypeptide having Genbank Accession Number NP_{—}001092458.1 (SEQ ID NO: 182);
the AQP11 polypeptide having Genbank Accession Number NP_{—}001103539.1 (SEQ ID NO: 184);
the CCDC126 polypeptide having Genbank Accession Number NP_{—}001075941.1 (SEQ ID NO: 186);
the KLF6 polypeptide having Genbank Accession Number NP_{—}001030348.2 (SEQ ID NO: 188);
the ACPP polypeptide having Genbank Accession Number NP_{—}001092336.1 (SEQ ID NO: 190);
the IGFBP4 polypeptide having Genbank Accession Number NP_{—}776982.1 (SEQ ID NO: 192);
the IGF1 polypeptide having Genbank Accession Number NP_{—}001071296.1 (SEQ ID NO: 194);
the IRS1 polypeptide having Genbank Accession Number XP_{—}581382.2 (SEQ ID NO: 196);
the IRS1 polypeptide having Genbank Accession Number XP_{—}002685688.1 (SEQ ID NO: 198);
the FOSL2 polypeptide having Genbank Accession Number NP_{—}001179879.1 (SEQ ID NO: 200);
the FOSL2 polypeptide having Genbank Accession Number XP_{—}002691497.1 (SEQ ID NO: 202);
the HRAS polypeptide having Genbank Accession Number XP_{—}590626.2 (SEQ ID NO: 204);
the HRAS polypeptide having Genbank Accession Number XP_{—}879748.1 (SEQ ID NO: 206);
the HRAS polypeptide having Genbank Accession Number XP_{—}879663.1 (SEQ ID NO: 208);
the CLU polypeptide having Genbank Accession Number NP 776327.1 (SEQ ID NO: 210);
the HSD17B1 polypeptide having Genbank Accession Number XP_{—}001253408.1 (SEQ ID NO: 212);
the HSD17B1 polypeptide having Genbank Accession Number NP_{—}001095835.1 (SEQ ID NO: 214);
the HSDL1 polypeptide having Genbank Accession Number NP_{—}001092341.1 (SEQ ID NO: 216);
the HSD11B2 polypeptide having Genbank Accession Number NP_{—}777067.1 (SEQ ID NO: 218);
the STC1 polypeptide having Genbank Accession Number NP_{—}788842.2 (SEQ ID NO: 220);
the CYP11A1 polypeptide having Genbank Accession Number NP_{—}788817.1 (SEQ ID NO: 222);
the GMNN polypeptide having Genbank Accession Number NP_{—}001020508.1 (SEQ ID NO: 224);
the CYP19A1 polypeptide having Genbank Accession Number NP_{—}776730.1 (SEQ ID NO: 226);
the IGFBP5 polypeptide having Genbank Accession Number NP_{—}001098797.1 (SEQ ID NO: 228);
the KCNK3 polypeptide having Genbank Accession Number XP_{—}597401.4 (SEQ ID NO: 230);
the KCNK3 polypeptide having Genbank Accession Number XP_{—}002691504.1 (SEQ ID NO: 232);
the SMAD7 polypeptide having Genbank Accession Number NP_{—}001179794.1 (SEQ ID NO: 234);
the SMAD7 polypeptide having Genbank Accession Number XP_{—}002697809.1 (SEQ ID NO: 236);
the SMAD7 polypeptide having Genbank Accession Number XP_{—}616030.3 (SEQ ID NO: 238);
the EGR3 polypeptide having Genbank Accession Number XP_{—}604596.4 (SEQ ID NO: 240);
the EGR3 polypeptide having Genbank Accession Number XP_{—}002689819.1 (SEQ ID NO: 242); and
the FN1 polypeptide having Genbank Accession Number NP_{—}001157250.1 (SEQ ID NO: 244).

70. The kit according to claim 67 wherein the domesticated mammal is a pig.

71. The kit according to claim 70 wherein the level of at least one of the following polypeptides is determined:
the KLF6 polypeptide having Genbank Accession Number NP_{—}001127825.1 (SEQ ID NO: 246);
the IGFBP4 polypeptide having Genbank Accession Number NP_{—}001116601.1 (SEQ ID NO: 248);
the IGF1 polypeptide having Genbank Accession Number NP_{—}999421.1 (SEQ ID NO: 250);
the HRAS polypeptide having Genbank Accession Number NP_{—}001038002.1 (SEQ ID NO: 252);
the CLU polypeptide having Genbank Accession Number NP_{—}999136.1 (SEQ ID NO: 254);
the HSD17B1 polypeptide having Genbank Accession Number NP_{—}001121944.1 (SEQ ID NO: 256);
the HSD11B2 polypeptide having Genbank Accession Number NP_{—}999078.1 (SEQ ID NO: 258);
the STC1 polypeptide having Genbank Accession Number NP_{—}001096682.1 (SEQ ID NO: 260);
the CYP11A1 polypeptide having Genbank Accession Number NP_{—}999592.1 (SEQ ID NO: 262);
the CYP19A1 polypeptide having Genbank Accession Number NP_{—}999594.1 (SEQ ID NO: 264);
the IGFBP5 polypeptide having Genbank Accession Number NP_{—}999264.1 (SEQ ID NO: 266);
the SMAD7 polypeptide having Genbank Accession Number XP_{—}001927617.1 (SEQ ID NO: 268);
the ACPP polypeptide having Genbank Accession Number XP_{—}003132467.1 (SEQ ID NO: 270);
the IRS1 polypeptide having Genbank Accession Number ACG59405.1 (SEQ ID NO: 272);
the EGR3 polypeptide having Genbank Accession Number XP_{—}003132855.1 (SEQ ID NO: 274);
the FN1 polypeptide having Genbank Accession Number XP_{—}003133689.1 (SEQ ID NO: 276);
the FN1 polypeptide having Genbank Accession Number XP_{—}003133690.1 (SEQ ID NO: 278); and
the FN1 polypeptide having Genbank Accession Number XP_{—}003133691.1 (SEQ ID NO: 280).

72. The kit according to claim 64 wherein the set of reagents detects polypeptides encoded by one or more marker genes of a human being.

73. The kit according to claim 72 wherein the set of reagents detects one or more of the following polypeptides.
the NEK6 polypeptide having Genbank Accession Number NP_{—}001138473.1 (SEQ ID NO: 74):
the NEK6 polypeptide having Genbank Accession Number NP_{—}001159639.1 (SEQ ID NO: 76);
the NEK6 polypeptide having Genbank Accession Number NP_{—}001159640.1 (SEQ ID NO: 78);
the NEK6 polypeptide having Genbank Accession Number NP_{—}001159641.1 (SEQ ID NO: 80);
the NEK6 polypeptide having Genbank Accession Number NP_{—}001159642.1 (SEQ ID NO: 82);
the NEK6 polypeptide having Genbank Accession Number NP_{—}001159643.1 (SEQ ID NO: 84);
the NEK6 polypeptide having Genbank Accession Number NP_{—}055212.2 (SEQ ID NO: 86);
the AQP11 polypeptide having Genbank Accession Number NP_{—}766627.1 (SEQ ID NO: 88);
the CCDC 126 polypeptide having Genbank Accession Number NP_{—}620126.2 (SEQ ID NO: 90);
the KLF6 polypeptide having Genbank Accession Number NP_{—}001153596.1 (SEQ ID NO: 92);
the KLF6 polypeptide having Genbank Accession Number NP_{—}001153597.1 (SEQ ID NO: 94);
the KLF6 polypeptide having Genbank Accession Number NP_{—}001291.3 (SEQ ID NO: 96);
the ACPP polypeptide having Genbank Accession Number NP_{—}001127666.1 (SEQ ID NO: 100);
the ACPP polypeptide having Genbank Accession Number NP_{—}001090.2 (SEQ ID NO: 98);
the IGFBP4 polypeptide having Genbank Accession Number NP_{—}001543.2 (SEQ ID NO: 102);
the IGF1 polypeptide having Genbank Accession Number NP_{—}000609.1 (SEQ ID NO: 104);
the IGF1 polypeptide having Genbank Accession Number NP_{—}001104753.1 (SEQ ID NO: 106);
the IGF1 polypeptide having Genbank Accession Number NP_{—}001104754.1 (SEQ ID NO: 108);
the IGF1 polypeptide having Genbank Accession Number NP_{—}001104755.1 (SEQ ID NO: 110);
the IRS1 polypeptide having Genbank Accession Number NP_{—}005535.1 (SEQ ID NO: 112);
the FOSL2 polypeptide having Genbank Accession Number NP_{—}005244.1 (SEQ ID NO: 114);
the HRAS polypeptide having Genbank Accession Number NP_{—}001123914.1 (SEQ ID NO: 116);
the HRAS polypeptide having Genbank Accession Number NP_{—}005334.1 (SEQ ID NO: 118);
the HRAS polypeptide having Genbank Accession Number NP_{—}789765.1 (SEQ ID NO: 120);
the CLU polypeptide having Genbank Accession Number NP_{—}001164609.1 (SEQ ID NO: 122);
the CLU polypeptide having Genbank Accession Number NP_{—}001822.2 (SEQ ID NO: 124);
the CLU polypeptide having Genbank Accession Number NP_{—}976084.1 (SEQ ID NO: 126);
the HSD17B1 polypeptide having Genbank Accession Number NP 000404.2 (SEQ ID NO: 128);
the HSDL1 polypeptide having Genbank Accession Number NP_{—}001139523.1 (SEQ ID NO: 130);
the HSDL1 polypeptide having Genbank Accession Number NP_{—}113651.4 (SEQ ID NO: 132);
the HSD11B2 polypeptide having Genbank Accession Number NP_{—}000187.3 (SEQ ID NO: 134);
the STC1 polypeptide having Genbank Accession Number NP_{—}003146.1 (SEQ ID NO: 136);
the CYP11A1 polypeptide having Genbank Accession Number NP_{—}000772.2 (SEQ ID NO: 138);
the CYP11A1 polypeptide having Genbank Accession Number NP_{—}001093243.1 (SEQ ID NO: 140);
the GMNN polypeptide having Genbank Accession Number NP_{—}056979.1 (SEQ ID NO: 142);
the HSD3B2 polypeptide having Genbank Accession Number NP_{—}000189.1 (SEQ ID NO: 144);
the HSD3B2 polypeptide having Genbank Accession Number NP_{—}001159592.1 (SEQ ID NO: 146);
the CYP19A1 polypeptide having Genbank Accession Number NP_{—}000094.2 (SEQ ID NO: 148);
the CYP19A1 polypeptide having Genbank Accession Number NP_{—}112503.1 (SEQ ID NO: 150);
the IGFBP5 polypeptide having Genbank Accession Number NP 000590.1 (SEQ ID NO: 152);
the KCNK3 polypeptide having Genbank Accession Number NP_{—}002237.1 (SEQ ID NO: 154);
the SMAD7 polypeptide having Genbank Accession Number NP_{—}001177750.1 (SEQ ID NO: 156);
the SMAD7 polypeptide having Genbank Accession Number NP_{—}001177751.1 (SEQ ID NO: 158);
the SMAD7 polypeptide having Genbank Accession Number NP_{—}001177752.1 (SEQ ID NO: 160);
the SMAD7 polypeptide having Genbank Accession Number NP_{—}005895.1 (SEQ ID NO: 162);
the EGR3 polypeptide having Genbank Accession Number NP 001186809.1 (SEQ ID NO: 164);
the EGR3 polypeptide having Genbank Accession Number NP_{—}001186810.1 (SEQ ID NO: 166);
the EGR3 polypeptide having Genbank Accession Number NP_{—}004421.2 (SEQ ID NO: 168);
the FN1 polypeptide having Genbank Accession Number NP_{—}002017.1 (SEQ ID NO: 170);
the FN1 polypeptide having Genbank Accession Number NP_{—}473375.2 (SEQ ID NO: 172);
the FN1 polypeptide having Genbank Accession Number NP_{—}997639.1 (SEQ ID NO: 174);
the FN1 polypeptide having Genbank Accession Number NP_{—}997641.1 (SEQ ID NO: 176);
the FN1 polypeptide having Genbank Accession Number NP_{—}997643.1 (SEQ ID NO: 178); and
the FN1 polypeptide having Genbank Accession Number NP_{—}997647.1 (SEQ ID NO: 180).
